(19) United States
(12) Patent Application Publication
(10) Pub. No.: US 2008/0300232 A1
(43) Pub. Date: Dec. 4, 2008

(54) N-Piperidine Derivatives as Ccr3 Modulators

(75) Inventors: Kay Brickmann, Molndal (SE); Bryan J. Egner, Molndal (SE); Fabrizio Giordanetto, Molndal (SE); Tord Inghardt, Molndal (SE); Anna Linusson Jonsson, Umea (SE); Fritiof Ponten, Molndal (SE)

Correspondence Address: Pepper Hamilton LLP, 400 Berwyn Park, 899 Cassatt Road, Berwyn, PA, 19312-1183, US

(73) Assignee: ASTRAZENECA AB, Sodertalje (SE)

(21) Appl. No.: 10/599,110

(22) PCT Filed: Mar. 21, 2005
(86) PCT No.: PCT/SE2005/000411

§ 371 Date: Dec. 4, 2006

(30) Foreign Application Priority Data
Mar. 22, 2004 (SE) 0400718-3
Nov. 12, 2004 (SE) 0402780-1

(51) Int. Cl. A61K 31/397; C07D 401/02; A61K 31/454; C07D 213/02; A61P 3/04; A61P 3/10; A61P 25/00; A61K 31/4545; C07D 239/24; A61K 31/506

(52) U.S. Cl. 514/210.2; 546/208; 514/326; 546/193; 514/318; 546/211; 544/242; 514/256

(57) ABSTRACT

Compounds of formula I, processes for preparing such compounds, their use in the treatment of obesity, psychiatric disorders, cognitive disorders, memory disorders, schizophrenia, epilepsy, and related conditions, and neurological disorders such as dementia, multiple sclerosis, Parkinson's disease, Huntington's chorea and Alzheimer's disease and pain related disorders and to pharmaceutical compositions containing them.

### FIELD OF INVENTION

The present invention relates to certain compounds of formula I-If, to processes for preparing such compounds, to their use in the treatment of obesity, psychiatric and neurological disorders, and to pharmaceutical compositions containing them.

### BACKGROUND OF THE INVENTION

Melanin concentrating hormone (MCH) is a cyclic peptide that was first isolated from fish over 15 years ago. In mammals, MCH gene expression is localised to the ventral aspect of the zona inserta and the lateral hypothalamic area (Breton et al., Molecular and Cellular Neurosciences, vol. 4, 271-284 (1993)). The latter region of the brain is associated with the control of behaviours such as eating and drinking, with arousal and with motor activity is (Baker, B., Trends Endocrinol. Metab. 5: 120-126 (1994), vol. 5, No. 3, 120-126 (1994)). Although the biological activity in mammals has not been fully defined, recent work has indicated that MCH promotes eating and weight gain (U.S. Pat. No. 5,849,708). Thus, MCH and its agonists have been proposed as treatments for anorexia nervosa and weight loss due to AIDS, renal disease, or chemotherapy. Similarly, antagonists of MCH can be used as a treatment for obesity and other disorders characterised by compulsive eating and excessive body weight. MCH projections are found throughout the brain, including the spinal cord, an area important in processing nociception, indicates that agents acting through MCH1r, such as compounds of formula I, will be useful in treating pain.

Two receptors for MCH (MCH1r (Shimomura et al. Biochem Biophys Res Commun 1999 Aug. 11; 261(3):622-6) & MCH2r (Hilol et al. J Biol. Chem. 2001 Jun. 8; 276(23):20125-9)) have been identified in humans, while only one (MCH1r) is present in rodent species (Tan et al. Genomics 2002 June; 79(6):785-92). In mice lacking MCH1r, there is no increased feeding response to MCH, and a lean phenotype is seen, suggesting that this receptor is responsible for mediating the feeding effect of MCH (Marsh et al. Proc. Natl. Acad. Sci. USA, 2002 Mar. 5; 99(5):3240-5). In addition, MCH receptor antagonists have been demonstrated to block the feeding effects of MCH (Takekawa et al. Eur. J. Pharmacol. 2002 Mar. 8; 438(3):129-35), and to reduce body weight & adiposity in diet-induced obese rats (Borowsky et al. Nature Med. 2002 August; 8(8):825-30). The conservation of distribution and sequence of MCH1r suggest a similar role for this receptor in man and rodent species. Hence, MCH receptor antagonists have been proposed as a treatment for obesity and other disorders characterised by excessive eating and body weight.

WO 03/106452 discloses certain 1-substituted-4-(substituted amino)piperidines which are alleged to be MCH-1r antagonists.

An abstract (No. 343 Vu V. Ma et al.,) from the 224^{th} ACS meeting in Boston, Mass., USA presents an MCH receptor antagonist for the potential treatment of obesity, with the following structure:

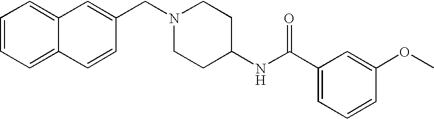

WO 01/14333 and GB 2 373 186 disclose that compounds of the following formula:

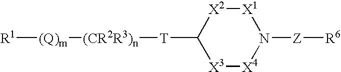

wherein

### Z is CR⁴R⁵, C(O) or CR⁴R⁵-Z¹;

Z¹ is C₁₋₄ alkylene (such as CH₂), C₂₋₄ alkenylene (such as CH═CH) or C(O)NH;
R¹ represents a C₁-C₁₂ alkyl group optionally substituted by one or more substituents independently selected from cyano, hydroxyl, C₁-C₆ alkoxy (such as methoxy or ethoxy), C₁-C₆ alkylthio (such as methylthio), C₃₋₇ cycloalkyl (such as cyclopropyl), C₁-C₆ alkoxycarbonyl (such as methoxycarbonyl) and phenyl (itself optionally substituted by one or more of halogen, nitro, cyano, C₁-C₆ alkyl, C₁-C₆ haloalkyl (such as CF₃), phenyl(C₁-C₆ alkyl) (such as benzyl), C₁-C₆ alkoxy, C₁-C₆ haloalkoxy, S(O)₂(C₁-C₆ alkyl), C(O)NH₂, carboxy or C₁-C₆ alkoxycarbonyl); or
R¹ represents C₂-C₆ alkenyl optionally substituted by phenyl (itself optionally substituted by one or more of halogen, nitro, cyano, C₁-C₆ alkyl, C₁-C₆ haloalkyl, phenyl(C₁-C₆ alkyl), C₁-C₆ alkoxy, C₁-C₆ haloalkoxy, S(O)₂(C₁-C₆ alkyl), C(O)NH₂, carboxy or C₁-C₆ alkoxycarbonyl); or
R¹ represents a 3- to 14-membered saturated or unsaturated ring system which optionally comprises up to two ring carbon atoms that form carbonyl groups and which optionally further comprises up to 4 ring heteroatoms independently selected from nitrogen, oxygen and sulphur, wherein the ring system is optionally substituted by one or more substituents independently selected from: halogen, cyano, nitro, oxo, hydroxyl, C₁-C₈ alkyl, C₁-C₆ hydroxyalkyl, C₁-C₆ haloalkyl, C₁₋₆ alkoxy(C₁-C₆ alkyl), C₃-C₇ cycloalkyl(C₁-C₆ alkyl), C₁-C₆ alkylthio(C₁-C₆ alkyl), C₁-C₆ alkylcarbonyloxy(C₁-C₆ alkyl), C₁-C₆ alkylS(O)₂(C₁-C₆ alkyl), aryl(C₁-C₆ alkyl), heterocyclyl(C₁-C₆ alkyl), arylS(O)₂(C₁-C₆ alkyl), heterocyclylS(O)₂(C₁-C₆ alkyl), aryl(C₁-C₆ alkyl)S(O)₂, heterocyclyl(C₁-C₆ alkyl)S(O)₂, C₂-C₆ alkenyl, C₁-C₆ alkoxy, carboxy-substituted C₁-C₆ alkoxy, C₁-C₆ haloalkoxy, C₁-C₆ hydroxyalkoxy, C₁-C₆ alkylcarboxy-substituted C₁-C₆ alkoxy, aryloxy, heterocyclyloxy, C₁-C₆ alkylthio, C₃-C₇ cycloalkyl(C₁-C₆ alkylthio), C₃-C₆ alkynylthio, C₁-C₆ alkylcarbonylamino, C₁-C₆ haloalkylcarbonylamino, SO₃H, —NR⁷R⁸, —C(O)NR²³R²⁴, S(O)₂NR¹⁸R¹⁹, S(O)₂R²⁰, R²⁵C(O), carboxyl, C₁-C₆ alkoxycarbonyl, aryl and heterocyclyl; wherein the foregoing aryl and heterocyclyl moieties are optionally substituted by one or more of halogen, oxo, hydroxy, nitro, cyano, C₁-C₆ alkyl, C₁-C₆ haloalkyl, phenyl(C₁-C₆ alkyl), C₁-C₆ alkoxy, C₁-C₆ haloalkoxy, S(O)₂(C₁-C₆ alkyl), C(O)NH₂, carboxy or C₁-C₆ alkoxycarbonyl;
m is 0 or 1;
Q represents an oxygen or sulphur atom or a group NR⁹, C(O), C(O)NR⁹, NR⁹C(O) or CH═CH;
n is 0, 1, 2, 3, 4, 5 or 6 provided that when n is 0, then m is 0;
each R² and R³ independently represents a hydrogen atom or a C₁-C₄ alkyl group, or (CR²R³)ₙ represents C₃-C₇ cycloalkyl optionally substituted by C₁-C₄ alkyl;
T represents a group NR¹⁰, C(O)NR¹⁰, NR¹¹C(O)NR¹⁰ or C(O)NR¹⁰NR¹¹;
X¹, X², X³ and X⁴ are, independently, CH₂, CHR¹² {wherein each R¹² is, independently, C₁-C₄ allyl or C₃-C₇ cycloalkyl(C₁-C₄ alkyl)} or C═O; or, when they are CHR¹², the R¹² groups of X¹ and X³ or X⁴, or, X² and X³ or X⁴ join to form a two or three atom chain which is CH₂CH₂, CH₂CH₂CH₂, CH₂OCH₂ or CH₂SCH₂; provided always that at least two of X¹, X², X³ and X⁴ are CH₂;
R⁴ and R⁵ each independently represent a hydrogen atom or a C₁-C₄ alkyl group;
R⁶ is aryl or heterocyclyl, both optionally substituted by one or more of: halogen, cyano, nitro, oxo, hydroxyl, C₁-C₈ alkyl, C₁-C₆ hydroxyalkyl, C₁-C₆ haloalkyl, C₁₋₆ alkoxy(C₁-C₆ alkyl), C₃-C₇ cycloalkyl(C₁-C₆ alkyl), C₁-C₆ alkylthio(C₁-C₆ alkyl), C₁-C₆ alkylcarbonyloxy(C₁-C₆ alkyl), C₁-C₆ alkylS(O)₂(C₁-C₆ alkyl), aryl(C₁-C₆ alkyl), heterocyclyl(C₁-C₆ alkyl), arylS(O)₂(C₁-C₆ alkyl), heterocyclylS(O)₂(C₁-C₆ alkyl), aryl(C₁-C₆ alkyl)S(O)₂, heterocyclyl(C₁-C₆ alkyl)S(O)₂, C₂-C₆ alkenyl, C₁-C₆ alkoxy, carboxy-substituted C₁-C₆ alkoxy, C₁-C₆ haloalkoxy, C₁-C₆ hydroxyalkoxy, C₁-C₆ alkylcarboxy-substituted C₁-C₆ alkoxy, aryloxy, heterocyclyloxy, C₁-C₆ alkylthio, C₃-C₇ cycloalkyl(C₁-C₆ alkylthio), C₃-C₆ alkynylthio, C₁-C₆ alkylcarbonylamino, C₁-C₆ haloalkylcarbonyl-amino, SO₃H, —NR¹⁶R¹⁷, —C(O)NR²¹R²², S(O)₂NR¹³R¹⁴, S(O)₂R¹⁵, R²⁶C(O), carboxyl, C₁-C₆ alkoxycarbonyl, aryl and heterocyclyl; wherein the foregoing aryl and heterocyclyl moieties are optionally substituted by one or more of halogen, nitro, cyano, C₁-C₆ alkyl, C₁-C₆ haloalkyl, phenyl(C₁-C₆ alkyl), C₁-C₆ alkoxy, C₁-C₆ haloalkoxy, S(O)₂(C₁-C₆ alkyl), C(O)NH₂, carboxy or C₁-C₆ alkoxycarbonyl;
R⁷, R⁸, R⁹, R¹⁰, R¹¹, R¹³, R¹⁴, R¹⁶, R¹⁷, R¹⁸, R¹⁹, R²¹, R²², R²³ and R²⁴ are, independently hydrogen, C₁-C₆ alkyl, C₁-C₆ haloalkyl, C₁-C₆ hydroxyalkyl, C₃-C₇ cycloalkyl, C₃-C₇ cycloalkyl(C₁-C₄ alkyl) or phenyl(C₁-C₆ alkyl); and,
R¹⁵ and R²⁰ are, independently, C₁-C₆ alkyl, C₁-C₆ hydroxyalkyl, C₃-C₆ cycloalkyl, C₃-C₇ cycloalkyl(C₁-C₄ alkyl) or C₁-C₆ alkyl optionally substituted by phenyl;
R²⁵ and R²⁶ are, independently, C₁-C₆ alkyl or phenyl (optionally substituted by one or more of halogen, nitro, cyano, C₁-C₆ alkyl, C₁-C₆ haloalkyl, phenyl(C₁-C₆ alkyl), C₁-C₆ alkoxy, C₁-C₆ haloalkoxy, S(O)₂(C₁-C₆ alkyl), C(O)NH₂, carboxy or C₁-C₆ alkoxycarbonyl);
or a pharmaceutically acceptable salt thereof, or solvate thereof, or a solvate of a salt thereof;
provided that when T is C(O)NR¹⁰ and R¹ is optionally substituted phenyl then n is not 0, have activity as modulators of chemokine receptor activity.

Compound 2-(4-chlorophenoxy)-N-{1-[4-(1,2,3-thiadiazol-4-yl)benzyl]piperidin-4-yl}-acetamide is specially disclosed. Hence, all compounds disclosed in these applications as examples are disclaimed from the compound claims of the present invention.

There is an unmet need for MCH receptor antagonists that are more potent, more selective, more bioavailable and produce less side effects than known compounds in this field.

### SUMMARY OF THE INVENTION

It is an object of the present invention to provide compounds, which are useful in treating obesity and related disorders, psychiatric disorders, neurological disorders and pain. This object has been reached in that a compound of formula I to If have been provided for use as a MCH receptor antagonist.

According to another aspect of the invention a pharmaceutical formulation is provided comprising a compound of formula I to If, and a pharmaceutically acceptable adjuvant, diluent or carrier.

According to a further aspect of the invention, the use of a compound of formula I to If is provided, in the preparation of a medicament for the treatment or prophylaxis of conditions associated with obesity.

According to yet another aspect of the invention, a method is provided of treating obesity, psychiatric disorders, anxiety, anxio-depressive disorders, depression, bipolar disorder, ADED, cognitive disorders, memory disorders, schizophrenia, epilepsy, and related conditions, and neurological disorders and pain related disorders, comprising administering a pharmacologically effective amount of a compound of Formula I to If to a patient in need thereof.

According to another aspect of the invention, a process for the preparation of compounds of formula I to If is provided.

According to a further aspect of the invention, a method is provided of treating obesity, type II diabetes, Metabolic syndrome and prevention of type II diabetes comprising administering a pharmacologically effective amount of a compound of formula I to If to a patient in need thereof.

Compounds of the present invention have the advantage that they may be more potent, more selective, more efficacious in vivo, be less toxic, be longer acting, produce fewer side effects, be more easily absorbed, be less metabolised and/or have a better pharmacokinetic profile than, or have other useful pharmacological or physicochemical properties over, compounds known in the prior art.

### DESCRIPTION OF THE INVENTION

The present invention relates to compounds of the general formula I

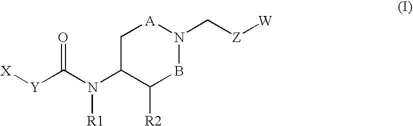

wherein X represents phenyl, naphthyl pyrrolyl, imidazolyl, furyl, thienyl, thiazolyl, isothiazolyl, thiadiazolyl, pyrazolyl, oxazolyl, isoxazolyl, pyridyl, pyrazinyl, pyrimidinyl, pyridazinyl, quinolinyl, isoquinolyl, quinazolyl, indolyl, benzofuranyl, benzo[b]thienyl or benzimidazolyl,
wherein each X is optionally substituted by one or more of the following: cyano, halo, a C₁₋₄ alkyl group optionally substituted by one or more fluoro, a C₁₋₄ alkoxy group optionally substituted by one or more fluoro, a group CONR^{a}R^{b} in which R^{a} and R^{b} independently represent a C₁₋₃ alkyl group, phenyl, phenoxy, 2-pyridyl or 3-pyridyl,
wherein the aromatic substituents (i.e. phenyl, phenoxy, 2-pyridyl or 3-pyridyl) may optionally be substituted by fluoro, chloro or cyano, or
X represents a diphenylmethyl or a dipyridinylmethyl group, optionally independently substituted at the aryl group(s) by one or more cyano, halo, trifluoromethoxy, difluoromethoxy or trifluoromethyl,
Y is OCH₂, SCH₂ (wherein the heteroatom is connected to X), CH₂CH₂ or CH═CH, wherein each carbon in Y is optionally substituted by 1 or 2 methyl groups and/or 1 or 2 fluoro,
R¹ represents H or a C₁₋₄alkyl group,
A represents (CH₂)ₙ, wherein n is 0 or 1 and B represents (CH₂)ₘ, wherein m is 0 or 1,
R² represents H or, when A and B are identical and represents CH₂, R₂ represents H or F,
Z represents phenyl or a heterocyclic group selected from thienyl, furyl, pyridyl, pyrazinyl, pyridazinyl, pyrrolyl, imidazolyl, thiazolyl, isothiazolyl, thiadiazolyl, pyrimidinyl, pyrazolyl, oxazolyl, isoxazolyl wherein each Z is optionally substituted by one or more of the following: cyano, halo, a C₁₋₄ alkyl group optionally substituted by one or more fluoro, a C₁₋₄ alkoxy group optionally substituted by one or more fluoro,
W represents phenyl or a heterocyclic group selected from thienyl, furyl, pyridyl, pyrazinyl, pyridazinyl, pyrrolyl, imidazolyl, thiazolyl, isothiazolyl, thiadiazolyl, pyrimidinyl, pyrazolyl, oxazolyl, isoxazolyl wherein each W is optionally substituted by one or more of the following: cyano, halo, a C₁₋₄ alkyl group optionally substituted by one or more fluoro, a C₁₋₄ alkoxy group optionally substituted by one or more fluoro, or W is optionally substituted with a trifluoromethylsulfonyl or a 2,2-difluoro-1,3-dioxolane ring (fused with two adjacent aromatic carbon atoms in W), as well as tautomers, optical isomers and racemates thereof as well as pharmaceutically acceptable salts thereof,
with the proviso that 2-(4-chlorophenoxy)-N-{1-[4-(1,2,3-thiadiazol-4-yl)benzyl]piperidin-4-yl}acetamide is excluded.

Particular groups now follow in which some of X, Y, Z, WI R¹ and R² in compounds of formula I are further defined. It will be understood that such group definitions may be used where appropriate with any of the other group definitions, claims or embodiments defined hereinbefore or hereinafter.

In one embodiment of the invention, X represents a phenyl or pyridyl group substituted with one or more cyano, fluoro, chloro, trifluoromethoxy, difluoromethoxy or trifluoromethyl, or X represents a diphenylmethyl or a dipyridinylmethyl group, optionally substituted at the aryl group(s) by one or more cyano, fluoro, chloro, trifluoromethoxy, difluoromethoxy or trifluoromethyl,

Y is OCH₂ or SCH₂ (both in which the heteroatom is connected to X), CH₂CH₂ or CH═CH,
R¹ is hydrogen or methyl
A represents (CH₂)ₙ, wherein n is 0 or 1 and B represents (CH₂)ₘ, wherein m is 0 or 1,
R² represents H or, when A and B are identical and represents CH₂, R₂ represents H or F,
Z is phenyl or a heterocyclic group selected from thienyl, furyl, pyrrolyl wherein each Z is optionally substituted by cyano, fluoro, chloro or trifluoromethyl,
W represents phenyl or a heterocyclic group selected from thienyl, furyl, pyridyl, pyrazinyl, pyridazinyl, pyrrolyl, imidazolyl, thiazolyl, isothiazolyl, thiadiazolyl, pyrimidinyl, pyrazolyl, oxazolyl, isoxazolyl wherein each W is optionally substituted by one or more of the following: cyano, fluoro, chloro, trifluoromethoxy, difluoromethoxy or trifluoromethyl, or with one trifluoromethylsulfomyl or one 2,2-difluoro-1,3-dioxolane ring (fused with two adjacent aromatic carbon atoms in W),
as well as pharmaceutically acceptable salts, thereof.

In another embodiment of the invention, X represents naphthyl, quinolinyl, isoquinolyl, quinazolyl, indolyl, benzofuranyl, benzo[b]thienyl, or benzimidazolyl, wherein each X is optionally substituted by one or more of the following: cyano, halo, a C₁₋₄ alkyl group optionally substituted by one or more fluoro, a C₁₋₄ alkoxy group optionally substituted by one or more fluoro, or a group CONR^{a}R^{b} in which R^{a} and R^{b} independently represent a C₁₋₃ alkyl group,

Y is OCH₂ or SCH₂ (wherein the heteroatom is connected to X), CH₂CH₂ or CH═CH,
R¹ is hydrogen or methyl,
A represents (CH₂)ₙ, wherein n is 0 or 1 and B represents (CH₂)ₘ, wherein m is 0 or 1,
R₂ represents H or, when A and B are identical and represents CH₂, R₂ represents H or F,
Z is phenyl or a heterocyclic group selected from, thienyl, furyl, pyrrolyl wherein each Z is optionally substituted by cyano, fluoro, chloro or trifluoromethyl,
W represents phenyl or a heterocyclic group selected from thienyl, furyl, pyridyl, pyrazinyl, pyridazinyl, pyrrolyl, imidazolyl, thiazolyl, isothiazolyl, thiadiazolyl, pyrimidinyl, pyrazolyl, oxazolyl, isoxazolyl wherein each W is optionally substituted by one or more of the following: cyano, fluoro, chloro, trifluoromethoxy, difluoromethoxy or trifluoromethyl, or with one trifluoromethylsulfonyl or one 2,2-difluoro-1,3-dioxolane ring (fused with two adjacent aromatic carbon atoms in W),
as well as pharmaceutically acceptable salts, thereof.

In yet another embodiment of the invention, X represents a phenyl or pyridyl group optionally substituted by one or more halogen and is further substituted by a phenyl, phenoxy, 2-pyridyl or 3-pyridyl group, wherein the substituents (i.e. phenyl, phenoxy, 2-pyridyl or 3-pyridyl) may optionally be further substituted by one or more fluoro, chloro or cyano

Y is OCH₂ or SCH₂ (wherein the heteroatom is connected to X), CH₂CH₂ or CH═CH,
R¹ is hydrogen or methyl,
A represents (CH₂)ₙ, wherein n is 0 or 1 and B represents (CH₂)ₘ, wherein m is 0 or 1,
R² represents H or, when A and B are identical and represents CH₂, R₂ represents H or F,
Z is phenyl or a heterocyclic group selected from thienyl, furyl, pyrrolyl wherein each Z is optionally substituted by cyano, fluoro, chloro or trifluoromethyl,
W represents phenyl or a heterocyclic group selected from thienyl, furyl, pyridyl, pyrazinyl, pyridazinyl, pyrrolyl, imidazolyl, thiazolyl, isothiazolyl, thiadiazolyl, pyrimidinyl, pyrazolyl, oxazolyl, isoxazolyl wherein each W is optionally substituted by one or more of the following: cyano, fluoro, chloro, trifluoromethoxy, difluoromethoxy or trifluoromethyl, or with one trifluoromethylsulfonyl or one 2,2-difluoro-1,3-dioxolane ring (fused with two adjacent aromatic carbon atoms in W), as well as pharmaceutically acceptable salts, thereof.

In one embodiment of the invention, X represents a phenyl group substituted with one or more cyano, fluoro, chloro, trifluoromethoxy, difluoromethoxy or trifluoromethyl, or X represents a diphenylmethyl or a dipyridinylmethyl group, optionally substituted at the aryl group(s) by one or more cyano, fluoro, chloro, trifluoromethoxy, difluoromethoxy or trifluoromethyl,

Y is OCH₂ (in which the heteroatom is connected to X),
R¹ is hydrogen,
A represents (CH₂)ₙ, wherein n is 0 or 1 and B represents (CH₂)ₘ, wherein m is 0 or 1,
R² represents H or, when A and B are identical and represents CH₂, R₂ represents H or F,
Z is thienyl, furyl or pyrrolyl,
W represents phenyl or a heterocyclic group selected from pyridyl, pyrazinyl, pyridazinyl, pyrrolyl, imidazolyl, thiazolyl, isothiazolyl, thiadiazolyl, pyrimidinyl, pyrazolyl, oxazolyl, isoxazolyl wherein each W is optionally substituted by one or more of the following: cyano, fluoro, chloro, trifluoromethoxy, difluoromethoxy or trifluoromethyl, or with one trifluoromethylsulfonyl or one 2,2-difluoro-1,3-dioxolane ring (fused with two adjacent aromatic carbon atoms in W),
as well as pharmaceutically acceptable salts thereof.

In a further embodiment of the invention, X represents a phenyl group substituted with one is or more cyano, fluoro, chloro, trifluoromethoxy, difluoromethoxy or trifluoromethyl, or X represents a diphenylmethyl group, optionally substituted at the phenyl group(s) by one or more cyano, fluoro, chloro, trifluoromethoxy, difluoromethoxy or trifluoromethyl,

Y is OCH₂ (in which the heteroatom is connected to X),
R¹ is hydrogen,

A represents (CH₂)ₙ, wherein n is 0 or 1 and B represents (CH₂)ₘ, wherein m is 0 or 1,

R² represents H or, when A and B are identical and represents CH₂, R₂ represents H or F,
Z is 2,5-thienyl (where position 2 is linked to group W),
W represents phenyl or a heterocyclic group selected from pyridyl, pyrazinyl, pyridazinyl, pyrrolyl, imidazolyl, thiazolyl, isothiazolyl, thiadiazolyl, pyrimidinyl, pyrazolyl, oxazolyl, isoxazolyl wherein each W is optionally substituted by one or more of the following: cyano, fluoro, chloro, trifluoromethoxy, difluoromethoxy or trifluoromethyl, or with one trifluoromethylsulfonyl or one 2,2-difluoro-1,3-dioxolane ring (fused with two adjacent aromatic carbon atoms in W),
as well as pharmaceutically acceptable salts thereof.

In another embodiment of the invention, X represents a phenyl group substituted with one or more cyano, fluoro, chloro, trifluoromethoxy, difluoromethoxy or trifluoromethyl, or X represents a diphenylmethyl group, optionally substituted (at the phenyl group(s)) by one or more cyano, fluoro, chloro, trifluoromethoxy, difluoromethoxy or trifluoromethyl,

Y is OCH₂ (in which the heteroatom is connected to X),
R¹ is hydrogen,
A represents (CH₂)ₙ, wherein n is 0 or 1 and B represents (CH₂)ₘ, wherein m is 0 or 1,
R² represents H or, when A and B are identical and represents CH₂, R₂ represents H or F,
Z is 2,5-furyl (where position 2 is linked to group W),
W represents phenyl or a heterocyclic group selected from pyridyl, pyrazinyl, pyridazinyl, pyrrolyl, imidazolyl, thiazolyl, isothiazolyl, thiadiazolyl, pyrimidinyl, pyrazolyl, oxazolyl, isoxazolyl wherein each W is optionally substituted by one or more of the following: cyano, fluoro, chloro, trifluoromethoxy, difluoromethoxy or trifluoromethyl, or with one trifluoromethylsulfonyl or one 2,2-difluoro-1,3-dioxolane ring (fused with two adjacent aromatic carbon atoms in W),
as well as pharmaceutically acceptable salts thereof.

In yet another embodiment of the invention, X represents a phenyl group substituted with one or more cyano, fluoro, chloro, trifluoromethoxy, difluoromethoxy or trifluoromethyl, or X represents a diphenylmethyl group, optionally substituted at the phenyl group(s) by one or more cyano, fluoro, chloro, trifluoromethoxy, difluoromethoxy or trifluoromethyl,

Y is OCH₂ (in which the heteroatom is connected to X),
R¹ is hydrogen,
A represents (CH₂)ₙ, wherein n is 0 or 1 and B represents (CH₂)ₘ, wherein m is 0 or 1,
R² represents H or, when A and B are identical and represents CH₂, R₂ represents H or F,
Z is 1, 3-1H pyrrolyl (in which the heteroatom is connected to W),
W represents phenyl or a heterocyclic group selected from pyridyl, pyrazinyl, pyridazinyl, pyrrolyl, imidazolyl, thiazolyl, isothiazolyl, thiadiazolyl, pyrimidinyl, pyrazolyl, oxazolyl, isoxazolyl wherein each W is optionally substituted by one or more of the following: cyano, fluoro, chloro, trifluoromethoxy, difluoromethoxy or trifluoromethyl, or with one trifluoromethylsulfonyl or one 2,2-difluoro-1,3-dioxolane ring (fused with two adjacent aromatic carbon atoms in W),
as well as pharmaceutically acceptable salts thereof.

In one group of compounds of formula I, Z is pyrrolyl and in another group of compounds, Z is 1, 3-1H pyrrolyl (in which the heteroatom is connected to W).

In yet another group of compounds of formula I, Y is OCH₂.

In a further group of compounds of Formula I, W is phenyl or 2-pyridyl, optionally substituted by one or more of the following: cyano, fluoro, chloro, trifluoromethoxy, difluoromethoxy, trifluoromethyl or trifluoromethylsulfonyl.

The invention also relates to compounds of the general formula Ia

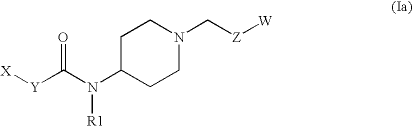

is wherein X represents a 5-10 membered aryl or a heterocyclic group selected from pyrrolyl, imidazolyl, furyl, thienyl, thiazolyl, isothiazolyl, thiadiazolyl, pyrazolyl, oxazolyl, isoxazolyl, pyridyl, pyrazinyl, pyrimidinyl, pyridazinyl, quinolinyl, isoquinolyl, quinazolyl, indolyl, benzofuranyl, benzo[b]thienyl, benzimidazolyl,
wherein each X is optionally substituted by one or more of the following: cyano, halo, a C₁₋₄ alkyl group optionally substituted by one or more fluoro, a C₁₋₄ alkoxy group optionally substituted by one or more fluoro, a group CONR^{a}R^{b} in which R^{a} and R^{b} independently represent a C₁₋₃ alkyl group, phenyl, phenoxy, 2-pyridyl or 3-pyridyl, wherein the aromatic substituents (i.e. phenyl, phenoxy, 2-pyridyl or 3-pyridyl) may optionally be substituted by fluoro, chloro or cyano,
Y is OCH₂, SCH₂ (both in which the heteroatom is connected to X), CH₂CH₂ or CH═CH, wherein each carbon in Y is optionally substituted by 1-2 methyl groups and/or 1-2 fluoride,
R¹ represents H or a C₁₋₄ alkyl group,
Z represents phenyl or a heterocyclic group selected from thienyl, furyl, pyridyl, pyrazinyl, pyridazinyl, pyrrolyl, imidazolyl, thiazolyl, isothiazolyl, thiadiazolyl, pyrimidinyl, pyrazolyl, oxazolyl, isoxazolyl wherein each Z is optionally substituted by one or more of the following: cyano, halo, a C₁₋₄ alkyl group optionally substituted by one or more fluoro, a C₁₋₄ alkoxy group optionally substituted by one or more fluoro,
W represents phenyl or a heterocyclic group selected from thienyl, furyl, pyridyl, pyrazinyl, pyridazinyl, pyrrolyl, imidazolyl, thiazolyl, isothiazolyl, thiadiazolyl, pyrimidinyl, pyrazolyl, oxazolyl, isoxazolyl wherein each W is optionally substituted by one or more of the following: cyano, halo, a C₁₋₄ alkyl group optionally substituted by one or more fluoro, a C₁₋₄ alkoxy group optionally substituted by one or more fluoro,
as well as tautomers, optical isomers and racemates thereof as well as pharmaceutically acceptable salts, thereof,
with the proviso that 2-(4-chlorophenoxy)-N-{1-[4-(1,2,3-thiadiazol-4-yl)benzyl]piperidin-4-yl}acetamide is excluded.

Particular groups now follow in which some of X, Y, Z, W, and R¹ in compounds of formula Ia are further defined. It will be understood that such group definitions may be used where appropriate with any of the other group definitions, claims or embodiments defined hereinbefore or hereinafter.

In a particular group of compounds of formula Ia, X represents a phenyl or pyridyl group substituted with one or more cyano, fluoro, chloro, trifluoromethoxy, difluoromethoxy or trifluoromethyl,
Y is OCH₂ or SCH₂ (both in which the heteroatom is connected to X) CH₂CH₂ or CH═CH,
R¹ is hydrogen or methyl,
Z is phenyl or a heterocyclic group selected from thienyl, furyl, pyridyl, pyrrolyl wherein each Z is optionally substituted by cyano, fluoro, chloro or trifluoromethyl,
W represents phenyl or a heterocyclic group selected from thienyl, furyl, pyridyl, pyrazinyl, pyridazinyl, pyrrolyl, imidazolyl, thiazolyl, isothiazolyl, thiadiazolyl, pyrimidinyl, pyrazolyl, oxazolyl, isoxazolyl wherein each W is optionally substituted by one or more of the following: cyano, fluoro, chloro, trifluoromethoxy, difluoromethoxy or trifluoromethyl,
as well as pharmaceutically acceptable salts thereof.

In another particular group of compounds of formula Ia, X represents naphthyl or a heteroaryl ring selected from quinolinyl, isoquinolyl, quinazolyl, indolyl, benzofuranyl, benzo[b]thienyl, or benzimidazolyl,

wherein each X is optionally substituted by one or more of the following: cyano, halo, a C₁₋₄ alkyl group optionally substituted by one or more fluoro, a C₁₋₄ alkoxy group optionally substituted by one or more fluoro, a group CONR^{a}R^{b} in which R^{a} and R^{b} independently represent a C₁₋₃ alkyl group,
Y is OCH₂ or SCH₂ (both in which the heteroatom is connected to X) CH₂CH₂ or CH═CH,
R¹ is hydrogen or methyl,
Z is phenyl or a heterocyclic group selected from thienyl, furyl, pyridyl, pyrrolyl wherein each Z is optionally substituted by cyano, fluoro, chloro or trifluoromethyl,
W represents phenyl or a heterocyclic group selected from thienyl, furyl, pyridyl, pyrazinyl, pyridazinyl, pyrrolyl, imidazolyl, thiazolyl, isothiazolyl, thiadiazolyl, pyrimidinyl, pyrazolyl, oxazolyl, isoxazolyl wherein each W is optionally substituted by one or more of the following: cyano, fluoro, chloro, trifluoromethoxy, difluoromethoxy or trifluoromethyl,
as well as pharmaceutically acceptable salts thereof.

In yet another group of compounds of formula Ia, X represents a phenyl or pyridyl group optionally substituted by on or more halogen and substituted by a phenyl, phenoxy, 2-pyridyl or 3-pyridyl group, wherein the substituents (i.e. phenyl, phenoxy, 2-pyridyl or 3-pyridyl) may optionally be further substituted by on or more fluoro, chloro or cyano,

Y is OCH₂ or SCH₂ (both in which the heteroatom is connected to X) CH₂CH₂ or CH═CH,
R¹ is hydrogen or methyl,
Z is phenyl or a heterocyclic group selected from thienyl, furyl, pyridyl, pyrrolyl wherein each Z is optionally substituted by cyano, fluoro, chloro or trifluoromethyl,
W represents phenyl or a heterocyclic group selected from thienyl, furyl, pyridyl, pyrazinyl, pyridazinyl, pyrrolyl, imidazolyl, thiazolyl, isothiazolyl, thiadiazolyl, pyrimidinyl, pyrazolyl, oxazolyl, isoxazolyl wherein each W is optionally substituted by one or more of the following: cyano, fluoro, chloro, trifluoromethoxy, difluoromethoxy or trifluoromethyl,
as well as pharmaceutically acceptable salts thereof.

In a further particular group of compounds of formula Ia, X represents a phenyl group substituted with one or more cyano, fluoro, chloro, trifluoromethoxy, difluoromethoxy or trifluoromethyl,

Y is preferably OCH₂ (in which the heteroatom is connected to X),
R¹ is hydrogen,
Z is thienyl, furyl or pyrrolyl,
W represents phenyl or a heterocyclic group selected from pyridyl, pyrazinyl, pyridazinyl, pyrrolyl, imidazolyl, thiazolyl, isothiazolyl, thiadiazolyl, pyrimidinyl, pyrazolyl, oxazolyl, isoxazolyl wherein each W is optionally substituted by one or more of the following: cyano, fluoro, chloro, trifluoromethoxy, difluoromethoxy or trifluoromethyl,
as well as pharmaceutically acceptable salts thereof.

In another particular group of compounds of formula Ia, X represents a phenyl group substituted with one or more cyano, fluoro, chloro, trifluoromethoxy, difluoromethoxy or trifluoromethyl,

Y is OCH₂ (in which the heteroatom is connected to X),
R¹ is hydrogen,
Z is 2,5-thienyl (where position 2 is linked to group W),
W represents phenyl or a heterocyclic group selected from pyridyl, pyrazinyl, pyridazinyl, pyrrolyl, imidazolyl, thiazolyl, isothiazolyl, thiadiazolyl, pyrimidinyl, pyrazolyl, oxazolyl, isoxazolyl wherein each W is optionally substituted by one or more of the following: cyano, fluoro, chloro, trifluoromethoxy, difluoromethoxy or trifluoromethyl,
as well as pharmaceutically acceptable salts thereof.

In a further particular group of compounds of formula Ia, X represents a phenyl group substituted with one or more cyano, fluoro, chloro, trifluoromethoxy, difluoromethoxy or trifluoromethyl,

Y is OCH₂ (in which the heteroatom is connected to X),
R¹ is hydrogen,
Z is 2,5-furyl (where position 2 is linked to group W),
W represents phenyl or a heterocyclic group selected from pyridyl, pyrazinyl, pyridazinyl, pyrrolyl, imidazolyl, thiazolyl, isothiazolyl, thiadiazolyl, pyrimidinyl, pyrazolyl, oxazolyl, isoxazolyl wherein each W is optionally substituted by one or more of the following: cyano, fluoro, chloro, trifluoromethoxy, difluoromethoxy or trifluoromethyl, as well as pharmaceutically acceptable salts thereof.

In another particular group of compounds of formula Ia, X represents a phenyl group substituted with one or more cyano, fluoro, chloro, trifluoromethoxy, difluoromethoxy or trifluoromethyl,

### Y is OCH₂

R¹ is hydrogen,
Z is 1, 3-1H pyrrolyl (in which the heteroatom is connected to W).
W represents phenyl or a heterocyclic group selected from pyridyl, pyrazinyl, pyridazinyl, pyrrolyl, imidazolyl, thiazolyl, isothiazolyl, thiadiazolyl, pyrimidinyl, pyrazolyl, oxazolyl, isoxazolyl wherein each W is optionally substituted by one or more of the following: cyano, fluoro, chloro, trifluoromethoxy, difluoromethoxy or trifluoromethyl,
as well as pharmaceutically acceptable salts thereof.

In one group of compounds of formula Ia, Z is pyrrolyl and in another group of compounds, Z is 1, 3-1H pyrrolyl (in which the heteroatom is connected to W).

In yet another group of compounds of formula Ia, Y is OCH₂.

In a further group of compounds of Formula Ia, W is phenyl or 2-pyridyl, optionally substituted by one or more of the following: cyano, fluoro, chloro, trifluoromethoxy, difluoromethoxy or trifluoromethyl.

The invention further relates to compounds of the general formula Ib

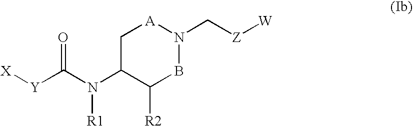

wherein X represents a diphenylmethyl or a dipyridinylmethyl group, optionally independently substituted (at the aryl group(s)) by one or more cyano, halo, trifluoromethoxy, difluoromethoxy or trifluoromethyl,
Y is OCH₂, SCH₂ (both in which the heteroatom is connected to X), CH₂CH₂ or CH═CH, wherein each carbon in Y is optionally substituted by 1 or 2 methyl groups and/or 1 or 2 fluoro,
R¹ represents H or a C₁₋₄alkyl group,
A represents (CH₂)ₙ, wherein n is 0 or 1 and B represents (CH₂)ₘ, wherein m is 0 or 1,
R₂ represents H or, when A and B are identical and represents CH₂, R₂ represents H or F,
Z represents phenyl or a heterocyclic group selected from thienyl, furyl, pyridyl, pyrazinyl, pyridazinyl, pyrrolyl, imidazolyl, thiazolyl, isothiazolyl, thiadiazolyl, pyrimidinyl, pyrazolyl, oxazolyl, isoxazolyl wherein each Z is optionally substituted by one or more of is the following: cyano, halo, a C₁₋₄ alkyl group optionally substituted by one or more fluoro, a C₁₋₄ alkoxy group optionally substituted by one or more fluoro,
W represents phenyl or a heterocyclic group selected from thienyl, furyl, pyridyl, pyrazinyl, pyridazinyl, pyrrolyl, imidazolyl, thiazolyl, isothiazolyl, thiadiazolyl, pyrimidinyl, pyrazolyl, oxazolyl, isoxazolyl wherein each W is optionally substituted by one or more of the following: cyano, halo, a C₁₋₄ alkyl group optionally substituted by one or more fluoro, a C₁₋₄ alkoxy group optionally substituted by one or more fluoro, or W is optionally substituted with a trifluoromethylsulfonyl or a 2,2-difluoro-1,3-dioxolane ring (fused with two adjacent aromatic carbon atoms in W),
as well as tautomers, optical isomers and racemates thereof as well as pharmaceutically acceptable salts thereof.

The invention further relates to compounds of the general formula Ic

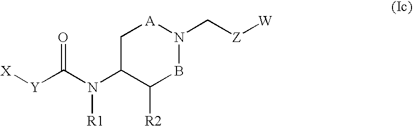

wherein X represents phenyl, naphtyl, pyrrolyl, imidazolyl, furyl, thienyl, thiazolyl, isothiazolyl, thiadiazolyl, pyrazolyl, oxazolyl, isoxazolyl, pyridyl, pyrazinyl, pyrimidinyl, pyridazinyl, quinolinyl, isoquinolyl, quinazolyl, indolyl, benzofuranyl, benzo[b]thienyl or benzimidazolyl,
wherein each X is optionally substituted by one or more of the following: cyano, halo, a C₁₋₄ alkyl group optionally substituted by one or more fluoro, a C₁₋₄ alkoxy group optionally substituted by one or more fluoro, a group CONR^{a}R^{b} in which R^{a} and R^{b} independently represent a C₁₋₃ alkyl group, phenyl, phenoxy, 2-pyridyl or 3-pyridyl, wherein the aromatic substituents (i.e. phenyl, phenoxy, 2-pyridyl or 3-pyridyl) may optionally be substituted by fluoro, chloro or cyano, or
X represents a diphenylmethyl or a dipyridinylmethyl group, optionally substituted at the aryl group(s) by one or more cyano, halo, trifluoromethoxy, difluoromethoxy or trifluoromethyl,
Y is OCH₂, SCH₂ (both in which the heteroatom is connected to X), CH₂CH₂ or CH═CH, wherein each carbon in Y is optionally substituted by 1 or 2 methyl groups and/or 1 or 2 fluoro,
R¹ represents H or a C₁₋₄alkyl group,
A represents (CH₂)ₙ, wherein n is 0 and B represents (CH₂)ₘ, wherein m is 0,
R² represents H,
Z represents phenyl or a heterocyclic group selected from thienyl, furyl, pyridyl, pyrazinyl, pyridazinyl, pyrrolyl, imidazolyl, thiazolyl, isothiazolyl, thiadiazolyl, pyrimidinyl, pyrazolyl, oxazolyl, isoxazolyl wherein each Z is optionally substituted by one or more of the following: cyano, halo, a C₁₋₄ alkyl group optionally substituted by one or more fluoro, a C₁₋₄ alkoxy group optionally substituted by one or more fluoro,
W represents phenyl or a heterocyclic group selected from thienyl, furyl, pyridyl, pyrazinyl, pyridazinyl, pyrrolyl, imidazolyl, thiazolyl, isothiazolyl, thiadiazolyl, pyrimidinyl, pyrazolyl, oxazolyl, isoxazolyl wherein each W is optionally substituted by one or more of the following: cyano, halo, a C₁₋₄ alkyl group optionally substituted by one or more fluoro, a C₁₋₄ alkoxy group optionally substituted by one or more fluoro, or W is optionally substituted with a trifluoromethylsulfonyl or a 2,2-difluoro-1,3-dioxolane ring (fused with two adjacent aromatic carbon atoms in W), as well as tautomers, optical isomers and racemates thereof as well as pharmaceutically acceptable salts thereof.

The invention further relates to compounds of the general formula Id

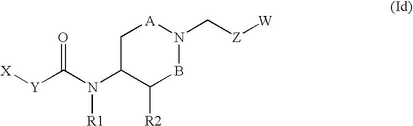

wherein X represents phenyl, naphtyl, pyrrolyl, imidazolyl, furyl, thienyl, thiazolyl, isothiazolyl, thiadiazolyl, pyrazolyl, oxazolyl, isoxazolyl, pyridyl, pyrazinyl, pyrimidinyl, pyridazinyl, quinolinyl, isoquinolyl, quinazolyl, indolyl, benzofuranyl, benzo[b]thienyl or benzimidazolyl,
wherein each X is optionally substituted by one or more of the following: cyano, halo, a C₁₋₄ alkyl group optionally substituted by one or more fluoro, a C₁₋₄ alkoxy group optionally substituted by one or more fluoro, a group CONR^{a}R^{b} in which R^{a} and R^{b} independently represent a C₁₋₃ alkyl group, phenyl, phenoxy, 2-pyridyl or 3-pyridyl, wherein the aromatic substituents (i.e. phenyl, phenoxy, 2-pyridyl or 3-pyridyl) may optionally be substituted by fluoro, chloro or cyano, or
X represents a diphenylmethyl or a dipyridinomethyl group, optionally substituted at the aryl group(s) by one or more cyano, halo, trifluoromethoxy, difluoromethoxy or trifluoromethyl,
Y is OCH₂, SCH₂ (both in which the heteroatom is connected to X), CH₂CH₂ or CH═CH, wherein each carbon in Y is optionally substituted by 1 or 2 methyl groups and/or 1 or 2 fluoro,
R¹ represents H or a C₁₋₄alkyl group,
A represents (CH₂)ₙ, wherein n is 0 and B represents (CH₂)ₘ, wherein m is 1, or vice versa,
R² represents H,
Z represents phenyl or a heterocyclic group selected from thienyl, furyl, pyridyl, pyrazinyl, pyridazinyl, pyrrolyl, imidazolyl, thiazolyl, isothiazolyl, thiadiazolyl, pyrimidinyl, pyrazolyl, oxazolyl, isoxazolyl wherein each Z is optionally substituted by one or more of the following: cyano, halo, a C₁₋₄ alkyl group optionally substituted by one or more fluoro, a C₁₋₄ alkoxy group optionally substituted by one or more fluoro,
W represents phenyl or a heterocyclic group selected from thienyl, furyl, pyridyl, pyrazinyl, pyridazinyl, pyrrolyl, imidazolyl, thiazolyl, isothiazolyl, thiadiazolyl, pyrimidinyl, pyrazolyl, oxazolyl, isoxazolyl wherein each W is optionally substituted by one or more of the following: cyano, halo, a C₁₋₄ alkyl group optionally substituted by one or more fluoro, a C₁₋₄ alkoxy group optionally substituted by one or more fluoro, or W is optionally substituted with a trifluoromethylsulfonyl or a 2,2-difluoro-1,3-dioxolane ring (fused with two adjacent aromatic carbon atoms in W), as well as tautomers, optical isomers and racemates thereof as well as pharmaceutically acceptable salts thereof.

The invention further relates to compounds of the general formula Ie

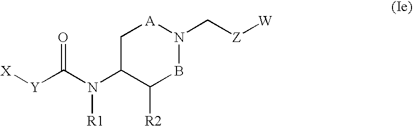

wherein X represents phenyl, naphtyl, pyrrolyl, imidazolyl, furyl, thienyl, thiazolyl, isothiazolyl, thiadiazolyl, pyrazolyl, oxazolyl, isoxazolyl, pyridyl, pyrazinyl, pyrimidinyl, pyridazinyl, quinolinyl, isoquinolyl, quinazolyl, indolyl, benzofuranyl, benzo[b]thienyl or benzimidazolyl,
wherein each X is optionally substituted by one or more of the following: cyano, halo, a C₁₋₄ alkyl group optionally substituted by one or more fluoro, a C₁₋₄ alkoxy group optionally substituted by one or more fluoro, a group CONR^{a}R^{b} in which R^{a} and R^{b} independently represent a C₁₋₃ alkyl group, phenyl, phenoxy, 2-pyridyl or 3-pyridyl, wherein the aromatic substituents (i.e. phenyl, phenoxy, 2-pyridyl or 3-pyridyl) may optionally be substituted by fluoro, chloro or cyano, or
X represents a diphenylmethyl or a dipyridinylmethyl group, optionally substituted at the aryl group(s) by one or more cyano, halo, trifluoromethoxy, difluoromethoxy or trifluoromethyl,
Y is OCH₂, SCH₂ (both in which the heteroatom is connected to X), CH₂CH₂ or CH═CH, wherein each carbon in Y is optionally substituted by 1 or 2 methyl groups and/or 1 or 2 fluoro,
R¹ represents H or a C₁₋₄alkyl group,
A and B both represents CH₂, R₂ represents F,
Z represents phenyl or a heterocyclic group selected from thienyl, furyl, pyridyl, pyrazinyl, pyridazinyl, pyrrolyl, imidazolyl, thiazolyl, isothiazolyl, thiadiazolyl, pyrimidinyl, pyrazolyl, oxazolyl, isoxazolyl wherein each Z is optionally substituted by one or more of the following: cyano, halo, a C₁₋₄ alkyl group optionally substituted by one or more fluoro, a C₁₋₄ alkoxy group optionally substituted by one or more fluoro,
W represents phenyl or a heterocyclic group selected from thienyl, furyl, pyridyl, pyrazinyl, pyridazinyl, pyrrolyl, imidazolyl, thiazolyl, isothiazolyl, thiadiazolyl, pyrimidinyl, pyrazolyl, oxazolyl, isoxazolyl wherein each W is optionally substituted by one or more of the following: cyano, halo, a C₁₋₄ alkyl group optionally substituted by one or more fluoro, a C₁₋₄ alkoxy group optionally substituted by one or more fluoro, or W is optionally substituted with a trifluoromethylsulfonyl or a 2,2-difluoro-1,3-dioxolane ring (fused with two adjacent aromatic carbon atoms in W), as well as tautomers, optical isomers and racemates thereof as well as pharmaceutically acceptable salts thereof.

The invention further relates to compounds of the general formula If

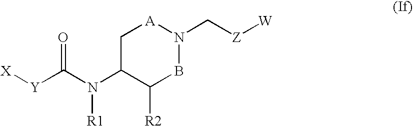

wherein X represents phenyl, naphtyl, pyrrolyl, imidazolyl, furyl, thienyl, thiazolyl, isothiazolyl, thiadiazolyl, pyrazolyl, oxazolyl, isoxazolyl, pyridyl, pyrazinyl, pyrimidinyl, pyridazinyl, quinolinyl, isoquinolyl, quinazolyl, indolyl, benzofuranyl, benzo[b]thienyl or benzimidazolyl,
wherein each X is optionally substituted by one or more of the following: cyano, halo, a C₁₋₄ alkyl group optionally substituted by one or more fluoro, a C₁₋₄ alkoxy group optionally substituted by one or more fluoro, a group CONR^{a}R^{b} in which R^{a} and R^{b} independently represent a C₁₋₃ alkyl group, phenyl, phenoxy, 2-pyridyl or 3-pyridyl, wherein the aromatic substituents (i.e. phenyl, phenoxy, 2-pyridyl or 3-pyridyl) may optionally be substituted by fluoro, chloro or cyano, or
X represents a diphenylmethyl or a dipyridinylmethyl group, optionally substituted at the aryl group(s) by one or more cyano, halo, trifluoromethoxy, difluoromethoxy or trifluoromethyl,
Y is OCH₂, SCH₂ (both in which the heteroatom is connected to X), CH₂CH₂ or CH═CH, wherein each carbon in Y is optionally substituted by 1 or 2 methyl groups and/or 1 or 2 fluoro,
R¹ represents H or a C₁₋₄alkyl group,
A represents (CH₂)ₙ, wherein n is 0 or 1 and B represents (CH₂)ₘ, wherein m is 0 or 1,
R₂ represents H or, when A and B are identical and represents CH₂, R₂ represents H or F,
Z represents phenyl or a heterocyclic group selected from thienyl, furyl, pyridyl, pyrazinyl, pyridazinyl, pyrrolyl, imidazolyl, thiazolyl, isothiazolyl, thiadiazolyl, pyrimidinyl, pyrazolyl, oxazolyl, isoxazolyl wherein each Z is optionally substituted by one or more of the following: cyano, halo, a C₁₋₄ alkyl group optionally substituted by one or more fluoro, a C₁₋₄ alkoxy group optionally substituted by one or more fluoro,
W represents phenyl or a heterocyclic group selected from thienyl, furyl, pyridyl, pyrazinyl, pyridazinyl, pyrrolyl, imidazolyl, thiazolyl, isothiazolyl, thiadiazolyl, pyrimidinyl, pyrazolyl, oxazolyl, isoxazolyl wherein each W is substituted by a trifluoromethylsulfonyl or a 2,2-difluoro-1,3-dioxolane ring (fused with two adjacent aromatic carbon atoms in W), as well as tautomers, optical isomers and racemates thereof as well as pharmaceutically acceptable salts thereof,

The term “pharmaceutically acceptable salt” refers to pharmaceutically acceptable acid addition salts. A suitable pharmaceutically acceptable salt of a compound of Formula I-If is, for example, an acid-addition salt of a compound of Formula I-If which is sufficiently basic, for example an acid-addition salt with an inorganic or organic acid such as:

(1S)-(+)-10-camphorsulfonic acid; cyclohexylsulfamic acid; phosphoric acid; dimethylphosphoric acid; p-toluenesulfonic acid; L-lysine; L-lysine hydrochloride; saccharinic acid; methanesulfonic acid; hydrobromic acid; hydrochloric acid; sulphuric acid; 1,2-ethanedisulfonic acid; (+/−)-camphorsulfonic acid; ethanesulfonic acid; nitric acid; p-xylenesulfonic acid; 2-mesitylenesulfonic acid; 1,5-naphthalenedisulfonic acid; 1-naphthalenesulfonic acid; 2-naphthalenesulfonic acid; benzenesulfonic acid; maleic acid; D-glutamic acid; L-glutamic acid; D,L-glutamic acid; L-arginine; glycine; salicylic acid; tartaric acid; fumaric acid; citric acid; L-(−)-malic acid; D,L-malic acid and D-gluconic acid.

Throughout the specification and the appended claims, a given chemical formula or name shall encompass all tautomers, all stereo and optical isomers and racemates thereof as well as mixtures in different proportions of the separate enantiomers, where such isomers and enantiomers exist, as well as pharmaceutically acceptable salts thereof. Isomers may be separated using conventional techniques, e.g. chromatography or fractional crystallisation. The enantiomers may be isolated by separation of racemate for example by fractional crystallisation, resolution or HPLC. The diastereomers may be isolated by separation of isomer mixtures for instance by fractional crystallisation, HPLC or flash chromatography. Alternatively the stereoisomers may be made by chiral synthesis from chiral starting materials under conditions, which will not cause racemisation or epimerisation, or by derivatisation, with a chiral reagent. All stereoisomers are included within the scope of the invention.

The following definitions shall apply throughout the specification and the appended claims.

Unless otherwise stated or indicated, the term “alkyl” denotes either a straight, branched or cyclic alkyl group. Examples of said alkyl include methyl, ethyl, n-propyl, isopropyl, cyclopropyl, n-butyl, iso-butyl, sec-butyl and t-butyl. Preferred alkyl groups are methyl, ethyl, propyl, isopropyl and tertiary butyl.

Unless otherwise stated or indicated, the term “alkoxy” denotes a group O-alkyl, wherein alkyl is as defined above.

Unless otherwise stated or indicated, the term “halo” shall mean fluorine, chlorine, bromine or iodine.

Specific compounds of the invention include one or more of the following:
2-(3-chlorophenoxy)-N-[1-[(1-phenyl-1H-pyrrol-3-yl)methyl]piperidin-4-yl}acetamide
2-(3-chlorophenoxy)-N-[1-({1-[4-(trifluoromethyl)phenyl]-1H-pyrrol-3-yl}methyl)piperidin-4-yl]acetamide
2-(3-chlorophenoxy)-N-(1-{[1-(4-methoxyphenyl)-1H-pyrrol-3-yl]methyl}piperidin-4-yl)acetamide
2-(3-chlorophenoxy)-N-(1-{[1-(2-chlorophenyl)-1H-pyrrol-3-yl]methyl}piperidin-4-yl)acetamide
2-(3-chlorophenoxy)-N-[1-({1-[5-(trifluoromethyl)pyridin-2-yl]-1H-pyrrol-3-yl}methyl)piperidin-4-yl]acetamide
2-(3-chlorophenoxy)-N-(1-{[1-(3-chlorophenyl)-1H-pyrrol-3-yl]methyl}piperidin-4-yl)acetamide
2-(3-chlorophenoxy)-N-[1-(4-pyridin-2-ylbenzyl)piperidin-4-yl]acetamide
2-(3-chlorophenoxy)-N-(1-{[5-(4-chlorophenyl)-2-furyl]methyl}piperidin-4-yl)acetamide
2-(3-chlorophenoxy)-N-[1-({1-[4-(trifluoromethoxy)phenyl]-1H-pyrrol-3-yl}methyl)piperidin-4-yl]acetamide
2-(3-chlorophenoxy)-N-{1-[3-(1H-pyrrol-1-yl)benzyl]piperidin-4-yl}acetamide
2-(3-chlorophenoxy)-N-[1-(3-pyridin-2-ylbenzyl)piperidin-4-yl]acetamide
2-(3-chlorophenoxy)-N-(1-{[5-(2,4-dichlorophenyl)-2-furyl]methyl}piperidin-4-yl)acetamide
2-(3-chlorophenoxy)-N-[1-({5-[1-methyl-5-(trifluoromethyl)-1H-pyrazol-3-yl]-2-thienyl}methyl)piperidin-4-yl]acetamide
N-(1-{[1-(4-bromophenyl)-1H-pyrrol-3-yl]methyl}piperidin-4-yl)-2-(3-chlorophenoxy)acetamide
2-(3-chlorophenoxy)-N-methyl-N-[1-({1-[4-(trifluoromethyl)phenyl]-1H-pyrrol-3-yl}methyl)piperidin-4-yl]acetamide
2-[(3-chlorophenyl)thio]-N-[1-({1-[4-(trifluoromethyl)phenyl]-1H-pyrrol-3-yl}methyl)piperidin-4-yl]acetamide
2-(pyridin-3-yloxy)-N-[1-({1-[4-(trifluoromethyl)phenyl]-1H-pyrrol-3-yl}methyl)piperidin-4-yl]acetamide
2-[3-(trifluoromethoxy)phenoxy]-N-[1-({1-[4-(trifluoromethyl)phenyl]-1H-pyrrol-3-yl}methyl)piperidin-4-yl]acetamide
2-[3-(trifluoromethoxy)phenoxy]-N-[1-({1-[5-(trifluoromethyl)pyridin-2-yl]-1H-pyrrol-3-yl}methyl)piperidin-4-yl]acetamide
2-(3-cyanophenoxy)-N-[1-({1-[4-(trifluoromethyl)phenyl]-1H-pyrrol-3-yl}methyl)piperidin-4-yl]acetamide
2-(3-fluorophenoxy)-N-[1-({1-[4-(trifluoromethyl)phenyl]-1H-pyrrol-3-yl}methyl)piperidin-4-yl]acetamide
2-(3-cyanophenoxy)-N-[1-({5-[1-methyl-5-(trifluoromethyl)-1H-pyrazol-3-yl]-2-thienyl}methyl)piperidin-4-yl]acetamide
2-(2-chlorophenoxy)-N-[1-({1-[4-(trifluoromethyl)phenyl]-1H-pyrrol-3-yl}methyl)piperidin-4-yl]acetamide
2-(3-chlorophenoxy)-N-[1-({5-[4-(trifluoromethoxy)phenyl]-2-furyl}methyl)piperidin-4-yl]acetamide
2-(3-chlorophenoxy)-N-(1-{[1-(4-cyanophenyl)-1H-pyrrol-3-yl]methyl}piperidin-4-yl)acetamide
2-(3-cyanophenoxy)-N-(1-{[5-(2,4-dichlorophenyl)-2-furyl]methyl}piperidin-4-yl)acetamide
2-(3-cyanophenoxy)-N-[1-({1-[4-(trifluoromethoxy)phenyl]-1H-pyrrol-3-yl}methyl)piperidin-4-yl]acetamide
2-(3-chlorophenoxy)-N-(1-{[1-(5-chloropyrimidin-2-yl)-1H-pyrrol-3-yl]methyl}piperidin-4-yl)acetamide
3-(3-chlorophenyl)-N-[1-({1-[4-(trifluoromethyl)phenyl]-1H-pyrrol-3-yl}methyl)piperidin-4-yl]propanamide
(2E)-3-(3-chlorophenyl)-N-[1-({1-[4-(trifluoromethyl)phenyl]-1H-pyrrol-3-yl}methyl)piperidin-4-yl]acrylamide
2-(3,5-difluorophenoxy)-N-[1-({1-[4-(trifluoromethyl)phenyl]-1H-pyrrol-3-yl}methyl)piperidin-4-yl]acetamide
2-(2,6-diisopropylphenoxy)-N-[1-({1-[4-(trifluoromethyl)phenyl]-1H-pyrrol-3-yl}methyl)piperidin-4-yl]acetamide
2-(3-isopropylphenoxy)-N-[1-({1-[4-(trifluoromethyl)phenyl]-1H-pyrrol-3-yl}methyl)piperidin-4-yl]acetamide
2-(2-cyanophenoxy)-N-[1-({1-[4-(trifluoromethyl)phenyl]-1H-pyrrol-3-yl}methyl)piperidin-4-yl]acetamide
2-(isoquinolin-5-yloxy)-N-[1-({1-[4-(trifluoromethyl)phenyl]-1H-pyrrol-3-yl}methyl)piperidin-4-yl]acetamide
2-(3,4-difluorophenoxy)-N-[1-({1-[4-(trifluoromethyl)phenyl]-1H-pyrrol-3-yl}methyl)piperidin-4-yl]acetamide
2-[(5-chloropyridin-2-yl)oxy]-N-[1-({1-[4-(trifluoromethyl)phenyl]-1H-pyrrol-3-yl}methyl)piperidin-4-yl]acetamide
2-(3-chlorophenoxy)-N-[1-({1-[6-(trifluoromethyl)pyridin-3-yl]-1H-pyrrol-3-yl}methyl)piperidin-4-yl]acetamide
2-(biphenyl-3-yloxy)-N-[1-({1-[4-(trifluoromethyl)phenyl]-1H-pyrrol-3-yl}methyl)piperidin-4-yl]acetamide,
2-(4-chlorophenoxy)-2-methyl-N-[1-({1-[4-(trifluoromethyl)phenyl]-1H-pyrrol-3-yl}methyl)piperidin-4-yl]propanamide,
2-(3-chlorophenoxy)-N-[1-({1-[4-(trifluoromethyl)phenyl]-1H-pyrrol-3-yl}methyl)azetidin-3-yl]acetamide
2-(diphenylmethoxy)-N-[1-({1-[4-(trifluoromethyl)phenyl]-1H-pyrrol-3-yl}methyl)piperidin-4-yl]acetamide
2-(3-chlorophenoxy)-N-[(3S,4S)-3-fluoro-1-({1-[4-(trifluoromethyl)phenyl]-1H-pyrrol-3-yl}methyl)piperidin-4-yl]acetamide
2-(3-chlorophenoxy)-N-[(3R,4R)-3-fluoro-1-({1-[4-(trifluoromethyl)phenyl]-1H-pyrrol-3-yl}methyl)piperidin-4-yl]acetamide
2-(3,4-difluorophenoxy)-N-[1-({1-[4-(trifluoromethyl)phenyl]-1H-pyrrol-3-yl}methyl)pyrrolidin-3-yl]acetamide
2-(3-chlorophenoxy)-N-{1-[(1-{4-[(trifluoromethyl)sulfonyl]plenyl}-1H-pyrrol-3-yl)methyl]piperidin-4-yl}acetamide
2-(3-chlorophenoxy)-N-(1-{[1-(2,2-difluoro-1,3-benzodioxol-5-yl)-1H-pyrrol-3-yl]methyl}piperidin-4-yl)acetamide
   and pharmaceutically acceptable salts thereof.

### Methods of Preparation

The compounds of the invention may be prepared as outlined below according to any of the following methods. However, the invention is not listed to these methods, the compounds may also be prepared as described for structurally related compounds in the prior art.

Compounds of formula I as well as Ia-If may be prepared by reacting a compound of formula II

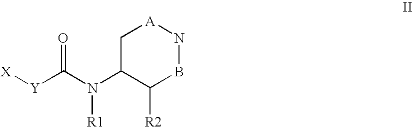

in which X, Y and R¹, R², A and B are as previously defined,
with a compound of formula III

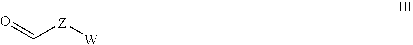

in which Z and W are as previously defined.

For example, a compound of formula II and a compound of formula III may be reacted together at a temperature in the range of 0° C. to 150° C., preferably in the range of 20° C. to 80° C. in the presence of a solvent, for example methanol, DCM, CHCl₃, THF or dioxane, in the presence of a reducing agent, for example sodium cyanoborohydride (optionally polymer supported) or sodium triacetoxyborohydride (optionally polymer supported). Optionally, a catalytic amount of an acid, e.g. acetic acid, may be added to the reaction mixture.

Alternatively, compounds of formula I as well as Ia-If may be prepared by reacting a compound of formula IV,

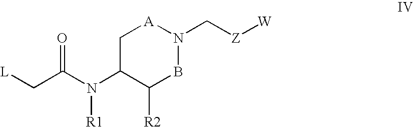

in which R¹, R², A, B, Z and W are as previously defined and where L is a leaving group such as halo or methanesulfonyloxy, with a compound of formula V

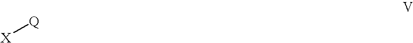

in which X is as previously defined and in which Q represents a hydroxy or a mercapto group.

For example, a compound of formula IV and a compound of formula V may be reacted together at a temperature in the range of 0° C. to 150° C., preferably in the range of 20° C. to 80° C. in the presence of a solvent, for example acetone, 2-butanone, dioxane, THF, DCM or 1,2-dichloroethane in the presence of a suitable inorganic or organic base, e.g. KOtBu, Cs₂CO₃, K₂CO₃ or NaH, optionally in the presence of a catayltic amount of KI or NaI.

Alternatively, compounds of formula I may be prepared by reacting a compound of formula VI,

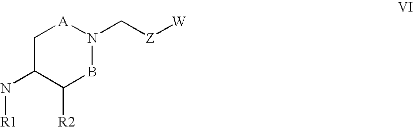

in which R¹, R², A, B, Z and W are as previously defined with a compound of formula VII

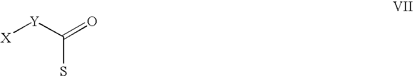

in which X and Y are as previously defined and in which S is a hydroxy group or a chlorine atom.

For example, a compound of formula VI and a compound of formula VII, in which S is a hydroxy group, may be reacted together at a temperature in the range of 0° C. to 150° C., preferably in the range of 20° C. to 80° C. in the presence of a solvent, for example THF, DCM, DCM/water (i.e. a two phase system) or DMF, optionally in the presence of a suitable inorganic or organic base, e.g. DIPEA or TEA, and a standard amide coupling reagent, e.g. HATU, TBTU, EDC, or DCC, the latter two of which may optionally be polymer supported.

Alternatively, compounds of formula I as well as Ia-If may be obtained by reaction of compounds of formula VII, in which S is chlorine, with compounds of formula VI in an inert solvent, e.g. THF, dioxane, DCM, CHCl₃ or 1,2-dichloroethane in the presence of a suitable inorganic or organic base, e.g. DIPEA, TEA, K₂CO₃ or NaHCO₃.

Compounds of formula II may be prepared by reacting a compound of formula VIII

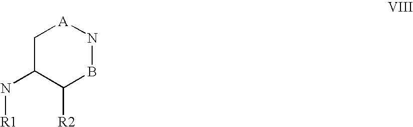

in which R¹, R², A, B are as previously defined, with a compound of formula VII e.g. by using one of the methods hereinbefore described for the reaction of compounds of formulae VI and VII.

Compounds of formula III, in which Z is a 1, 3-1H-pyrrolyl ring, may be prepared by reaction of a compound of formula IX with a compound of formula X in which W is as previously defined.

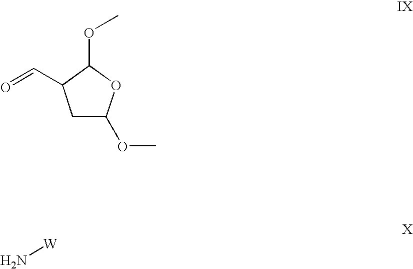

For example, a compound of formula IX and a compound of formula X may be reacted together at a temperature in the range of 20° C. to 90° C. in acetic acid.

Alternatively, compounds of formula III may be prepared by reaction of a compound of formula XI, in which Z is as previously defined and in which T is bromine or iodine with a compound of formula XII in which W is as previously defined.

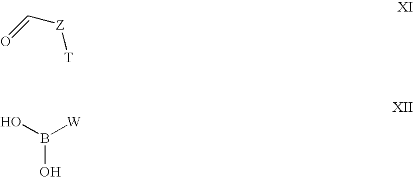

For example, a compound of formula XI and a compound of formula XII may be reacted together under palladium catalysis using a method described e.g. in Feuerstein, M et al., Tetrahedr. Lett. 42 (33), 5659, 2001.

Alternatively, using similar synthetic methodology, compounds of formula II may be prepared by reaction of a compound of formula XIII, in which Z is as previously defined with a compound of formula XIV in which W and T are as previously defined

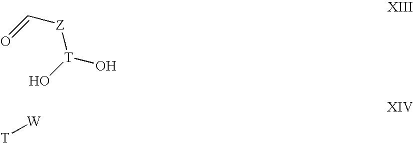

Compounds of formula IV may be prepared by reacting a compound of formula VI with a compound of formula XV, wherein L and S are as previously described, e.g. by using one of the methods hereinbefore described for the reaction of compounds of formulae VI and VII.

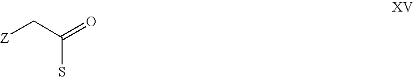

Compounds of formula VIII, in which R² represents a fluorine atom (and A and B are both representing CH₂) may be prepared starting with fluorination (using e.g. SELECTFLUOR™ Reagent) of the silyl enol ether of piperidone, as described e.g. by van Neil, M. B. et al. J. Med. Chem. 1999, 42, 2087-2104, followed by reductive amination of the so formed α-fluoro piperidone, e.g. as described hereinafter in the Experimental Section.

Compounds of formula V, VII, VIII and IX-XV are either commercially available or can be prepared by methods well known to those skilled in the art.

Optionally, the ring nitrogen in formula VIII may be protected prior to reaction with a compound of formula VII. Amine protecting groups are known to those skilled in the art, for example the benzyl, t-Boc, or Cbz groups.

The compounds of the invention may be isolated from their reaction mixtures using conventional techniques. Stereoisomers may be separated using conventional techniques, e.g. chromatography or fractional crystallisation. Enantiomers may be isolated by separation of racemate for example by fractional crystallisation, resolution or HPLC. The diastereomers may be isolated by separation of isomer mixtures for instance by fractional crystallisation, HPLC or flash chromatography. Alternatively the stereoisomers may be made by chiral synthesis from chiral starting materials under conditions which will not cause racemisation or epimerisation, or by derivatisation, with a chiral reagent.

Persons skilled in the art will appreciate that, in order to obtain compounds of the invention in an alternative and in some occasions, more convenient manner, the individual process steps mentioned hereinbefore may be performed in a different order, and/or the individual reactions may be performed at a different stage in the overall route (i.e. chemical transformations may be performed upon different intermediates to those associated hereinbefore with a particular reaction).

Certain compounds of formulae II, III, IV and VI are novel and are claimed as a further aspect of the present invention as useful intermediates:
2-(3-chlorophenoxy)-N-piperidin-4-ylacetamide
2-(3-cyanophenoxy)-N-piperidin-4-ylacetamide
2-(3-fluorophenoxy)-N-piperidin-4-ylacetamide
2-(2-chlorophenoxy)-N-piperidin-4-ylacetamide
N-piperidin-4-yl-2-(pyridin-3-yloxy)acetamide
N-piperidin-4-yl-2-[3-(trifluoromethoxy)phenoxy]acetamide
2-phenoxy-N-piperidin-4-ylacetamide
2-(3-chlorophenoxy)-N-methyl-N-piperidin-4-ylacetamide
2-[(3-chlorophenyl)thio]-N-piperidin-4-ylacetamide
1-[5-(trifluoromethyl)pyridin-2-yl]-1H-pyrrole-3-carbaldehyde
1-(5-chloropyrimidin-2-yl)-1H-pyrrole-3-carbaldehyde
4-(3-formyl-1H-pyrrol-1-yl)benzonitrile
2-chloro-N-[1-({1-[4-(trifluoromethyl)phenyl]-1H-pyrrol-3-yl}methyl)piperidin-4-yl]acetamide
1-({1-[4-(trifluoromethyl)phenyl]-1H-pyrrol-3-yl}methyl)piperidin-4-amine dihydrochloride
tert-butyl[1-({1-[4-(trifluoromethyl)phenyl]-1H-pyrrol-3-yl}methyl)piperidin-4-yl]carbamate
1-(6-trifluoromethyl-pyridin-3-yl)-1H-pyrrole-3-carbaldehyde
2-(3,4-difluorophenoxy)-N-pyrrolidin-3-ylacetamide
1-(2,2-difluoro-benzo[1,3]dioxol-5-yl)-1H-pyrrole-3-carbaldehyde
1-(4-trifluoromethanesulfonyl-phenyl)-1H-pyrrole-3-carbaldehyde

The compounds of the invention may be isolated from their reaction mixtures using conventional techniques. Persons skilled in the art will appreciate that, in order to obtain compounds of the invention in an alternative and in some occasions, more convenient manner, the individual process steps mentioned hereinbefore may be performed in a different order, and/or the individual reactions may be performed at a different stage in the overall route (i.e. chemical transformations may be performed upon different intermediates to those associated hereinbefore with a particular reaction). The expression “inert solvent” refers to a solvent, which does not react with the starting materials, reagents, intermediates or products in a manner, which adversely affects the yield of the desired product.

### Pharmaceutical Preparations

The compounds of the invention will normally be administered via the oral, parenteral, intravenous, intramuscular, subcutaneous or in other injectable ways, buccal, rectal, vaginal, transdermal and/or nasal route and/or via inhalation, in the form of pharmaceutical preparations comprising the active ingredient either as a free base, or a pharmaceutically acceptable inorganic or organic addition salt, in a pharmaceutically acceptable dosage form. Depending upon the disorder and patient to be treated and the route of administration, the compositions may be administered at varying doses.

Suitable daily doses of the compounds of the invention in the therapeutic treatment of humans are about 0.001-10 mg/kg body weight, preferably 0.01-3 mg/kg body weight.

Oral formulations are preferred particularly tablets or capsules which may be formulated by methods known to those skilled in the art to provide doses of the active compound in the range of 0.5 mg to 500 mg for example 1 mg, 3 mg, 5 mg, 10 mg, 25 mg, 50 mg, 100 mg and 250 mg.

According to a further aspect of the invention there is also provided a pharmaceutical formulation including any of the compounds of the invention, or pharmaceutically acceptable derivatives thereof, in admixture with pharmaceutically acceptable adjuvants, diluents and/or carriers.

The compounds of the invention may also be combined with other therapeutic agents, which are useful in the treatment of disorders associated with obesity, psychiatric disorders, neurological disorders and pain.

### Pharmacological Properties

The compounds of formula I-If are useful for the treatment of obesity, psychiatric disorders such as psychotic disorders, anxiety, anxio-depressive disorders, depression, cognitive disorders, memory disorders, schizophrenia, epilepsy, and related conditions, and neurological disorders such as dementia, multiple sclerosis, Raynaud's syndrome, Parkinson's disease, Huntington's chorea and Alzheimer's disease. The compounds are also potentially useful for the treatment of immune, cardiovascular, reproductive and endocrine disorders, and diseases related to the respiratory and gastrointestinal systems. The compounds are also potentially useful as agents for ceasing consumption of tobacco, treating nicotine dependence and/or treating nicotine withdrawal symptoms, reducing the craving for nicotine and as anti-smoking agents. The compounds may also eliminate the increase in weight that normally accompanies the cessation of smoking. The compounds are also potentially useful as agents for treating or preventing diarrhea.

The compounds are also potentially useful as agents for reducing the craving/relapse for addictive substances that include, but are not limited to psychomotor-active agents such as nicotine, alcohol, cocaine, amphetamines, opiates, benzodiazepines and barbiturates. The compounds are also potentially useful as agents for treating drug addiction and/or drug abuse.

Accordingly, it is desirable to provide a compound and method of treatment which will be active in reducing craving for the abused substance, and which does not exacerbate the sympathetic response rate caused by the abused substance and which has favourable pharmacodynamic effects.

The compounds are also potentially useful as agents for treating pain disorders, including but not limited to acute and chronic nociceptive, inflammatory and neuropathic pain and migraine.

In another aspect the present invention provides a compound of Formula I-If for use as a medicament.

In a further aspect the present invention provides the use of a compound of Formula I-If in the preparation of a medicament for the treatment or prophylaxis of obesity, psychiatric disorders such as psychotic disorders, anxiety, anxio-depressive disorders, depression, bipolar disorder, ADHD, cognitive disorders, memory disorders, schizophrenia, epilepsy, and related conditions, neurological disorders such as dementia, multiple sclerosis, Parkinson's disease, Huntington's chorea and Alzheimer's disease and pain related disorders, including but not limited to acute and chronic nociceptive, inflammatory and neuropathic pain and migraine, comprising administering a pharmacologically effective amount of a compound of Formula I-If to a patient in need thereof.

In a still further aspect the present invention provides a method of treating obesity, psychiatric disorders such as psychotic disorders, anxiety, anxio-depressive disorders, depression, bipolar disorder, ADID, cognitive disorders, memory disorders, schizophrenia, epilepsy, and related conditions, and neurological disorders such as dementia, multiple sclerosis, Parkinson's disease, Huntington's chorea and Alzheimer's disease and pain related disorders, including but not limited to acute and chronic nociceptive, inflammatory and neuropathic pain and migraine, comprising administering a pharmacologically effective amount of a compound of Formula I-If to a patient in need thereof.

The compounds of the present invention are particularly suitable for the treatment of obesity, e.g. by reduction of appetite and body weight, maintenance of weight reduction and prevention of rebound. The compounds of the present invention may also be used to prevent or reverse medication-induced weight gain, e.g. weight gain caused by antipsychotic (neuroleptic) treatment(s). The compounds of the present invention may also be used to prevent or reverse weight gain associated with smoking cessation

In another aspect the present invention provides a method of treating obesity, type II diabetes, Metabolic syndrome and a method of preventing type II diabetes comprising administering a pharmacologically effective amount of a compound of Formula I-If to a patient in need thereof.

### Combination Therapy

The compounds of the invention may be combined with another therapeutic agent that is useful in the treatment of disorders associated with the development and progress of atherosclerosis such as hypertension, hyperlipidaemias, dyslipidaemias, diabetes and obesity. For example, a compound of the present invention may be used in combination with a compound that affects thermogenesis, lipolysis, fat absorption, satiety, or gut motility. The compounds of the invention may be combined with another therapeutic agent that decreases the ratio of LDL:HDL or an agent that causes a decrease in circulating levels of LDL-cholesterol. In patients with diabetes mellitus the compounds of the invention may also be combined with therapeutic agents used to treat complications related to micro-angiopathies.

The compounds of the invention may be used alongside other therapies for the treatment of metabolic syndrome or type 2 diabetes and its associated complications; these include biguanide drugs, insulin (synthetic insulin analogues), oral antihyperglycemics (these are divided into prandial glucose regulators and alpha-glucosidase inhibitors) and PPAR modulating agents.

In another aspect of the invention, the compound of Formula I-If, or a pharmaceutically acceptable salt, solvate, solvate of such a salt or a prodrug thereof, may be administered in association with a PPAR modulating agent for example tesaglitazar. PPAR modulating agents include but are not limited to a PPAR alpha and/or gamma agonist, or pharmaceutically acceptable salts, solvates, solvates of such salts or prodrugs thereof. Suitable PPAR alpha and/or gamma agonists, pharmaceutically acceptable salts, solvates, solvates of such salts or prodrugs thereof are well known in the art.

In addition the combination of the invention may be used in conjunction with a sulfonylurea. The present invention also includes a compound of the present invention in combination with a cholesterol-lowering agent. The cholesterol-lowering agents referred to in this application include but are not limited to inhibitors of HMG-CoA reductase (3-hydroxy-3-methylglutaryl coenzyme A reductase). Suitably the HMG-CoA reductase inhibitor is a statin for example rosuvastatin.

In the present application, the term “cholesterol-lowering agent” also includes chemical modifications of the HMG-CoA reductase inhibitors, such as esters, prodrugs and metabolites, whether active or inactive.

The present invention also includes a compound of the present invention in combination with an inhibitor of the ileal bile acid transport system (IBAT inhibitor). The present invention also includes a compound of the present invention in combination with a bile acid binding resin.

According to an additional further aspect of the present invention there is provided a combination treatment comprising the administration of an effective amount of a compound of the Formula I-If, or a pharmaceutically acceptable salt, solvate, solvate of such a salt or a prodrug thereof, optionally together with a pharmaceutically acceptable diluent or carrier, with the simultaneous, sequential or separate administration one or more of the following agents selected from:

a CETP (cholesteryl ester transfer protein) inhibitor;
a cholesterol absorption antagonist;
a MTP (microsomal transfer protein) inhibitor;
a nicotinic acid derivative, including slow release and combination products;
a phytosterol compound;
probucol;
an anti-obesity compound, for example orlistat (EP 129,748) and sibutramine (GB 2,184,122 and U.S. Pat. No. 4,929,629);
an antihypertensive compound, for example an angiotensin converting enzyme (ACE) inhibitor for example lisinopril and ramipril, an angiotensin II receptor antagonist, an andrenergic blocker, an alpha andrenergic blocker, a beta andrenergic blocker for example metoprolol and metoprolol succinate, a mixed alphalbeta andrenergic blocker, an andrenergic stimulant, calcium channel blocker for example felodipine, an AT-1 receptor blocker for example candesartan and candesartan cilexetil, a saluretic, a diuretic or a vasodilator;
a CB 1 antagonist or inverse agonist, for example rimonabant;
another melanin concentrating hormone (MCH) antagonist;
a PDK inhibitor; or
modulators of nuclear receptors for example LXR, FXR, RSR, and RORalpha;

### an SSRI;

a serotonin antagonist;
or a pharmaceutically acceptable salt, solvate, solvate of such a salt or a prodrug thereof, optionally together with a pharmaceutically acceptable diluent or carrier to a warm-blooded animal, such as man in need of such therapeutic treatment.

Therefore in an additional feature of the invention, there is provided a method for the treatment of type 2 diabetes and its associated complications in a warm-blooded animal, such as man, in need of such treatment which comprises administering to said animal an effective amount of a compound of Formula I-If, or a pharmaceutically acceptable salt, solvate, solvate of such a salt or a prodrug thereof in simultaneous, sequential or separate administration with an effective amount of a compound from one of the other classes of compounds described in this combination section, or a pharmaceutically acceptable salt, solvate, solvate of such a salt or a prodrug thereof.

Therefore in an additional feature of the invention, there is provided a method of treating hyperlipidemic conditions in a warm-blooded animal, such as man, in need of such treatment which comprises administering to said animal an effective amount of a compound of Formula I-If, or a pharmaceutically acceptable salt, solvate, solvate of such a salt or a prodrug thereof in simultaneous, sequential or separate administration with an effective amount of a compound from one of the other classes of compounds described in this combination section or a pharmaceutically acceptable salt, solvate, solvate of such a salt or a prodrug thereof.

According to a further aspect of the invention there is provided a pharmaceutical composition which comprises a compound of Formula I-If, or a pharmaceutically acceptable salt, solvate, solvate of such a salt or a prodrug thereof, and a compound from one of the other classes of compounds described in this combination section or a pharmaceutically acceptable salt, solvate, solvate of such a salt or a prodrug thereof, in association with a pharmaceutically acceptable diluent or carrier.

According to a further aspect of the present invention there is provided a kit comprising a compound of Formula I-If, or a pharmaceutically acceptable salt, solvate, solvate of such a salt or a prodrug thereof, and a compound from one of the other classes of compounds described in this combination section or a pharmaceutically acceptable salt, solvate, solvate of such a salt or a prodrug thereof.

According to a further aspect of the present invention there is provided a kit comprising:

a) a compound of Formula I-If, or a pharmaceutically acceptable salt, solvate, solvate of such a salt or a prodrug thereof, in a first unit dosage form;
b) a compound from one of the other classes of compounds described in this combination section or a pharmaceutically acceptable salt, solvate, solvate of such a salt or a prodrug thereof; in a second unit dosage form; and
c) container means for containing said first and second dosage forms.

According to a further aspect of the present invention there is provided a kit comprising:

a) a compound of Formula I-If, or a pharmaceutically acceptable salt, solvate, solvate of such a salt or a prodrug thereof, together with a pharmaceutically acceptable diluent or carrier, in a first unit dosage form;
b) a compound from one of the other classes of compounds described in this combination section or a pharmaceutically acceptable salt, solvate, solvate of such a salt or a prodrug thereof, in a second unit dosage form; and
c) container means for containing said first and second dosage forms.

According to another feature of the invention there is provided the use of a compound of the Formula I-If, or a pharmaceutically acceptable salt, solvate, solvate of such a salt or a prodrug thereof, and one of the other compounds described in this combination section, or a pharmaceutically acceptable salt, solvate, solvate of such a salt or a prodrug thereof, in the manufacture of a medicament for use in the treatment of metabolic syndrome or type 2 diabetes and its associated complications in a warm-blooded animal, such as man.

According to another feature of the invention there is provided the use of a compound of the Formula I-If, or a pharmaceutically acceptable salt, solvate, solvate of such a salt or a prodrug thereof, and one of the other compounds described in this combination section, or a pharmaceutically acceptable salt, solvate, solvate of such a salt or a prodrug thereof, in the manufacture of a medicament for use in the treatment of hyperlipidaemic conditions in a warm-blooded animal, such as man.

According to a further aspect of the present invention there is provided a combination treatment comprising the administration of an effective amount of a compound of the Formula I-If, or a pharmaceutically acceptable salt, solvate, solvate of such a salt or a prodrug thereof, optionally together with a pharmaceutically acceptable diluent or carrier, with the simultaneous, sequential or separate administration of an effective amount of one of the other compounds described in this combination section, or a pharmaceutically acceptable salt, solvate, solvate of such a salt or a prodrug thereof, optionally together with a pharmaceutically acceptable diluent or carrier to a warm-blooded animal, such as man in need of such therapeutic treatment.

### Experimental Section

The invention will now be described in more detail with the following examples that are not to be construed as limiting the invention.

### ABBREVIATIONS

aq. aqueous
Ac acetyl
Bu butyl
tBoc tert-butyloxycarbonyl
Cbz benzyloxycarbonyl
CHO Chinese hamster ovary (cells)
DCM dichloromethane
DIPEA di-isopropyl ethyl amine
DMA dimethyl acetamide
DMF N,N-dimethylformamide
DTT dithiothreitol
EDC.HCl 1-ethyl-3-(3-dimethylaminopropyl)carbodiimide hydrochloride
EDTA ethylenediamine tetraacetic acid
ELS evaporative light scattering
ESI electrospray ionization
Et ethyl
GDP guanosine 5′-diphosphate
HATU O-(azabenzotriazol-1-yl)-N,N,N′,N′-tetramethyluronium hexafluoro-phosphate
HEK human embryotic kidney (cells)
HEPES N-2-hydroxyethyl piperazine-N′-2-ethanesulfonic acid
HPLC high performance liquid chromatography
LC liquid chromatography
MP-BH(OAc)₃ macroporous polymer bound triacetoxyborohydride (available from Argonaut)
MS mass spectroscopy
Pol-BH₃CN (polystyrylmethyl)trimethylammonium cyanoborohydride (loading 4.1-4.3 mmol BH₃CN/g)
Pol-CHO 4-benzyloxybenzaldehyde polystyrene (loading ˜2.66 mmol CHO/g)
SELECTFLUOR™ Reagent: 1-chloromethyl-4-fluoro-1,4-diazoniabicyclo[2.2.2]octan bis(tetrafluoroborate)
TBTU N,N,N′,N′-tetramethyl-O-(benzotriazol-1-yl)uronium tetrafluoroborate
TEA triethylamine
TFA trifluoroacetic acid
THF tetrahydrofuran
TLC thin layer chromatography
TMSCl chloro(trimethyl)silane
Tris trishydroxymethylaminomethane
Tween polyoxyethylene sorbitan monolaurate
t tert
rt. room temperature
sat. saturated
br broad
bs broad singlet
d doublet
dd doublet of doublets
m multiplet
q quartet
s singlet
t triplet

### General Experimental Procedures

Flash column chromatography employed MERCK normal phase silica gel 60 Å (40-63 μm) or a Biotage Horizon Pioneer® JPFC system equipped with FLASH 12+M or FLASH 25+M or 40+M silica cartridges. Mass spectra were recorded on a Waters Micromass ZQ single quadrupole equipped with a pneumatically assisted electrospray, interface (LC-MS).

HPLC analyses were performed on a Gynkotek P580 HPG, gradient pump with a Gynkotek UVD 170S UV-Vis detector. Column: Chromolith Performance RP-18e, 4.6×100 mm, Mobile phase A: Acetonitrile, Mobile phase B: 0.1% TFA (aq), Flow: 3 ml/min, Injection volume: 20 μl, Detection: 254 and 275 nm.

Purifications were performed on a semi preparative HPLC, Shimadzu LC-8A, Shimadzu SPD-10A UV-vis. detector equipped with a Waters X-terra® Prep MS C₁₈ Column, 250 mm×50 mm (10 μm) or on a Waters Pxep LC 2000 with UV-detection, equipped with a Kromasil 10 μm C8 250 mm×20 mm column, or on a semi preparative HPLC, Shimadzu LC-8A, Shimadzu SPD-10A UV-vis.-detector equipped with a Waters Symmetry® 100 mm×19 mm C18 5 μm column.

Automated HPLC purification was done using a Waters Fraction Lynx system equipped with V, ELS and MS detection and an Ace C8 5μ 10 cm×21.2 id column. The mobile phase was A: 95% CH₃CN and B: 5% CH₃CN+95% 0.1 M NH₄OAc with a gradient from 100% B to 100% A in 10 minutes at 25 mL/min flow rate.

¹H NMR and ¹³C NMR spectra were obtained at 298 K on a Varian Unity Plus 400 mHz, or a Varian Inova 500 MHz or a Varian Unity Plus 600 MHz or a Bruker Avance 300 MHz or Varian Gemini 2000 300 MHz. Chemical shifts are given in ppm with the solvent residual peak as internal standard: CDCl₃ δ_{H} 7.26, δ_{C} 77.2; MeOH-d₄ δ_{H} 3.31, δ_{C} 49.0; DMSO-d₆ δ_{H} 2.50; δ_{C} 39.5 ppm.

Microwave heating was performed using single node heating in a Smith Creator from Personal Chemistry, Uppsala, Sweden.

Chemical names (IUPAC) were generated using the software ACD/Name version 6.00.

Names/reference numbers of starting materials (CAS no), either commercially available or prepared according to literature procedures.

5-[4-(trifluoromethoxy)phenyl]-2-furaldehyde, 306935-95-5; 5-(2,4-dichlorophenyl)-2-furaldehyde, 56300-69-7; tert-butyl piperidin-4-ylcarbamate, 73874-95-0; 3-amino-pyrrolidine-1-carboxylic acid tert-butyl ester, 186550-13-0; 3-(3-chlorophenyl)propanoic acid, 21640-48-2; (2E)-3-(3-chlorophenyl)acrylic acid, 14473-90-6; chloroacetic acid, 79-11-8; 3,5-difluorophenol, 2713-34-0; 2-hydroxybenzonitrile, 611-20-1; isoquinolin-5-ol, 2439-04-5; 2,6-di-isopropylphenol, 2078-54-8; 3-isopropylphenol, 618-45-1; 4-aminobenzotrifluoride, 455-14-1; 4-amino-benzonitrile, 873-74-5; 5-formyl-2-furylboronic acid, 27329-70-0; 2-amino-5-chloropyrimidine, 5428-89-7; 4-pyridin-2-ylbenzaldehyde, 127406-56-8; 5-(4-chlorophenyl)-2-furaldehyde, 34035-03-5; 1-[4-(trifluoromethoxy)phenyl]-1H-pyrrole-3-carbaldehyde, 439094-17-4; 3-(1H-pyrrol-1-yl)benzaldehyde, 129747-77-9; 3-pyridin-2-ylbenzaldehyde, 85553-53-3; 5-(2,4-dichlorophenyl)-2-furaldehyde, 56300-69-7; 1-(4-bromophenyl)-1H-pyrrole-3-carbaldehyde, 477850-19-4; 5-[1-methyl-5-(trifluoromethyl)-1H-pyrazol-3-yl]thiophene-2-carbaldehyde, 175202-93-4; aniline, 62-53-3; 1-benzylpiperidin-4-amine, 50541-93-0; chloroacetyl chloride, 74-04-9; 2-chloroaniline, 95-51-2; 3-chloroaniline, 108-42-9; 1-chloroethyl chloroformate, 50893-53-3; 2-chlorophenol, 95-57-8; 3-chlorophenol, 108-43-0; 2,5-dimethoxy-3-tetrahydrofurancarboxaldehyde, 50634-05-4; 3-fluorophenol, 372-20-3; 108-43-0; 3-hydroxy-benzonitrile, 873-62-1; 5-trifluoromethyl-pyridine-2-ylamine, 74784-70-6; 3-hydroxypyridine, 109-00-2; 3-chlorothiophenol, 2037-31-2; phenol, 108-95-2; tert-butyl 4-aminopiperidine-1-carboxylate, 87120-72-7; 3-(trifluoromethoxy)phenol, 827-99-6; 4-methoxyaniline, 104-94-9; 3-amino-6-(trifluoromethyl)pyridine, 106877-33-2; 3,4-difluorophenol, 2713-33-9; 3-phenylphenol, 580-51-8; 2-chloro-5-hydroxypyridine, 41288-96-4; 3-chlorophenol, 108-43-0; 2-(4-chlorophenoxy)-2-methylpropanoic acid, 882-09-7; tert-butyl 4-oxopiperidine-1-carboxylate, 79099-07-3; chloro(trimethyl)silane, 75-77-4; (3-chlorophenoxy)acetic acid, 588-32-9; Selectfluor Reagent, 140681-55-6, chloroacetic acid, 79-11-8; 3-chlorophenol, 108-43-0; 3,4-difluoro-phenol, 2713-33-9; tert-butyl azetidin-3-yl carbamate, 91188-13-5; 4-(trifluoromethylsulfonyl)aniline, 473-27-8; 2,2-difluoro-benzo[1,3]dioxol-5-ylalmine, 1544-85-0

### Preparation of Intermediates

### EXAMPLE A

### 2-(3-chlorophenoxy)-N-piperidin-4-ylacetamide

### i) N-(1-benzylpiperidin-4-yl)-2-chloroacetamide

Chloroacetyl chloride (1.68 mL, 21.1 mmol) was added dropwise to a stirred solution of 1-benzylpiperidin-4-amine (3.65 g, 19.2 mmol) in DCM (65 mL). The mixture was stirred for 2 h at rt. whereafter additional DCM (100 mL) was added. The organic phase was washed with NaHCO₃ (3×100 mL, aq., sat.), dried over MgSO₄ and concentrated to give 4.43 g (86%) of the title compound as an off-white solid. This material was used in the next step without further purification.

¹H NMR (DMSO-d₆) δ 8.11 (br d, 1H), 7.20-7.35 (m, 5H), 4.00 (s, 2H), 3.53 (m, 1H), 3.44 (s, 2H), 2.73 (m, 2H), 2.00, (m, 2H), 1.69 (m, 2H), 1.34-1.48 (m, 2H). MS (ESI) 267 (M+H⁺).

### ii) N-(1-benzylpiperidin-4-yl)-2-(3-chlorophenoxy)acetamide

Potassium tert-butoxide (2.24 g, 19.0 mmol) was added portionwise to a solution of 3-chlorophenol (2.33 g, 18.1 mmol) in THF (75 mL) and the mixture was stirred at rt. until a clear solution was obtained. N-(1-benzylpiperidin-4-yl)-2-chloroacetamide (4.39 g, 16.5 mmol) dissolved in THF (50 mL) was added dropwise over 10 minutes and the mixture was stirred for 4 h after which additional potassium tert-butoxide (0.2 g, 1.8 mmol) was added followed by further stirring at rt. for 1 h. Water (50 mL) was added and the mixture was concentrated. The aqueous residue was extracted with EtOAc (3×75 mL) and the combined organic phases were washed with 1 M NaOH (75 mL). The organic phase was concentrated and the residue was purified on silica gel eluted with DCM:MeOH (98:2) to give 5.15 g (87%) of the title compound as a off-white solid.

¹H NMR (DMSO-d₆) δ 7.96 (br d, 1H), 7.22-7.35 (m, 6H), 6.99-7.04 (m, 2H), 6.93 (m, 1H), 4.49 (s, 2H), 3.63 (m, 1H), 3.44 (s, 2H), 2.74 (m, 2H), 1.99 (m, 2H), 1.68 (m, 2H), 1.43-1.55 (m, 2H). MS (ESI) 360 (M+H⁺).

### iii) 2-(3-chlorophenoxy)-N-piperidin-4-ylacetamide

1-Chloroethyl chloroformate (2.04 g, 14.3 mmol) was added to a solution of N-(1-benzylpiperidin-4-yl)-2-(3-chlorophenoxy)acetamide (4.1 g, 11.4 mmol) in dichloroethane (70 mL) and the mixture was heated at reflux for 1 h. The reaction mixture was concentrated and methanol (70 mL) was added and heated to reflux for 17 h (over night). The reaction mixture was concentrated and the residue was dissolved in HCl diluted with water (100 mL) and extracted with Et₂O (2×75 mL). The aqueous phase was made basic with 2M NaOH and extracted with EtOAc (2×150 mL). The combined organic phases were concentrated and the residue was purified on silica gel eluted with first DCM:MeOH (9:1) followed by DCM:MeOH (8:2) containing 1% TEA and finally with DCM:MeOH (7:3) containing 1% TEA, to give 2.25 g (73%) of the title compound.

¹H NMR (DMSO-d₆) δ 7.97 (br d, 1H), 7.31 (t, 1H), 7.02-7.05 (m, 2H), 6.92 (dd, 1H), 4.49 (s, 2H), 3.66 (m, 1H), 2.90 (m, 2H), 2.46 (m, 2H), 1.62 (m, 2H), 1.24-1.39 (m, 2H).

MS (ESI) 269 (M+H⁺).

Using the method described in Example A, the compounds of Examples B and D were similarly prepared from N-(1-benzylpiperidin-4-yl)-2-chloroacetamide and the appropriate phenols:

### EXAMPLE B

### 2-(3-cyanophenoxy)-N-piperidin-4-ylacetamide

The crude product was purified on silica gel eluted with first DCM:MeOH (9:1) followed by DCM:MeOH (8:2) containing 1% TEA and finally with DCM:MeOH (7:3) containing 1% TEA, to give the title compound in 34% yield (two steps).

¹H NMR (CDCl₃, conformer mixture, * denotes minor conformer peaks) δ 7.43 (t, 1H), 7.33 (br d, 1H), 7.13-7.25 (m, 2H), 6.49 (d br, 1H), 6.34* (d br, 1H), 4.49 (s, 2H), 4.01 (m, 1H), 3.89* (m, 1H), 3.12-3.25 (m, 3H), 2.85-2.95* (m, 2H), 2.70-2.82 (m, 2H), 2.06-2.16* (m, 2H), 1.88-2.03 (m, 2H), 1.42-1.58 (m, 2H).

¹³C NMR (CDCl₃, conformer mixture, * denotes minor conformer peaks) δ 166.4, 157.3, 130.9, 126.1, 119.6, 119.5*, 118.4, 118.3*, 113.8, 67.6, 53.5*, 50.6*, 46.9*, 46.4, 45.0, 32.5, 32.2*.

MS (ESI) 260.2 (M+H⁺).

### EXAMPLE C

### 2-(3-fluorophenoxy)-N-piperidin-4-ylacetamide

The crude product was purified on silica gel eluted with first DCM:MeOH (9:1) followed by DCM:MeOH (9:1) containing 1% NH₃ (aq.) and finally with DCM:MeOH (8:2) containing 1% NH₃ (aq.), to give the title compound in 61% overall yield (two steps).

¹H NMR (CDCl₃) δ 7.27 (m, 1H), 6.78-6.64 (m, 3H), 6.41 (br d, 1H), 4.45 (s, 2H), 3.97 (m, 1H), 3.07 (m, 2H), 2.71 (m, 2H), 1.95 (m, 2H), 1.76 (m, 4H), 1.44-1.31 (m, 2H). ¹³C NMR (CDCl₃) δ 166.9, 163.7 (d, J=247 Hz), 158.4 (d, J=11 Hz), 130.8 (d, J=10 Hz), 110.3, 109.2 (d, J=21 Hz), 102.9 (d, J=26 Hz), 67.6, 46.7, 45.4, 33.3.

MS (ESI) 253.3 (M+H⁺).

### EXAMPLE D

### 2-(2-chlorophenoxy)-N-piperidin-4-ylacetamide

The crude product was purified on silica gel eluted with first DCM:MeOH (9:1) followed by DCM:MeOH (8:2) containing 1% TEA to give the title compound in 24% overall yield (two steps):

¹H NMR (CDCl₃) δ 7.37-7.39 (m, 1H), 7.20-7.26 (m, 1H), 6.95-7.00 (m, 2H), 6.87-6.89 (m, 1H), 4.52 (s, 2H), 4.09 (m, 1H), 3.40-3.60 (m, 3H), 3.03 (m, 2H), 2.20 (m, 2H), 1.92-2.0 (m, 2H).

¹³C NMR (CDCl₃) δ 167.5, 152.8, 130.6, 128.3, 123.3, 123.2, 114.4, 68.3, 44.3, 43.0, 28.6.

MS (ESI) 269.2 (M+H⁺).

### EXAMPLE E

### N-piperidin-4-yl-2-(pyridin-3-yloxy)acetamide

### i) tert-butyl 4-[(chloroacetyl)amino]piperidine-1-carboxylate

A mixture of tert-butyl 4-aminopiperidine-1-carboxylate (5.0 g, 25 mmol) and chloroacetyl chloride (3.1 g, 27.5 mmol) in DCM (50 mL) was stirred at rt. under N₂ atmosphere until TLC indicated that starting material was consumed (2.5 h). The mixture was diluted with DCM and washed with sat. aq. NaHCO₃. The organic layer was separated and the solvent was removed. The residue was purified on silica gel eluted with DCM:MeOH (9:1) to give 6 g (87%) of the title compound.

¹H NMR (CDCl₃) δ 6.47 (br s, 1H), 3.86-4.16 (m, 5H), 2.79-2.96 (m, 2H), 1.82-2.0 (m, 2H), 1.28-1.53 (m, 11H).

MS (ESI) 277 (M+H⁺).

### ii) tert-butyl 4-{[(pyridin-3-yloxy)acetyl]amino}piperidine-1-carboxylate

Potassium tert-butoxide (1.14 g, 10.1 mmol) was added to a solution of 3-hydroxypyridine (1.03 g, 10.8 mmol) in THF (50 mL) and the mixture was stirred at rt for 20 minutes. tert-Butyl 4-[(chloroacetyl)amino]piperidine-1-carboxylate (2.0 g, 7.2 mmol) in THF (20 mL) was added dropwise over 5 minutes and the mixture was stirred at rt. until LC-MS indicated that starting material was consumed. The mixture was concentrated and the residue was dissolved in H₂O (100 mL) and subsequently extracted with EtOAc (3×70 mL). The combined organic phases were washed with brine (60 mL), dried (Na₂SO₄) and concentrated. The residue was purified on silica gel eluted with DCM:MeOH (9:1) to give 1.01 g (42%) of the title compound.

¹H NMR (CDCl₃) δ 8.41-8.24 (m, 2H), 7.32-7.18 (m, 2H), 6.43 (br d, J=7.5 Hz, 1H), 4.52 (s, 2H), 4.18-3.95 (m, 3H), 2.87 (m, 2H), 1.93 (m, 2H), 1.45 (s, 9H), 1.50-1.30 (m, 2H).

MS (ESI) 336 (M+H⁺).

### iii) N-piperidin-4-yl-2-(pyridin-3-yloxy)acetamide

To a solution of tert-butyl 4-{[(pyridin-3-yloxy)acetyl]amino}piperidine-1-carboxylate (1.01 g, 3.0 mmol) in DCM (30 mL) was added TFA (5 mL) and the mixture was stirred at rt. until LC-MS indicated that starting material was consumed. The reaction mixture was concentrated and the residue was dissolved in EtOAc (200 mL) and washed with 1M NaOH (2×50 mL) and brine (50 mL). After drying (Na₂SO₄) the organic phase was evaporated to dryness. The aqueous phase was extracted with DCM (3×80 mL) and the combined organic phases were washed with brine, dried (Na₂SO₄) and evaporated. The combined residues were dissolved in DCM, filtered and evaporated. The residue was purified on silica gel eluted with DCM:MeOH:NEt₃ (gradient from 90:10:1 to 60:40:1) to give 0.46 g (65%) of the title compound as a sticky oil. The material was solidified by treatment with DCM/Et₂O followed by evaporation.

¹H NMR (MeOD-d₄) δ 8.32 (d, J=2.4 Hz, 1H), 8.18 (m, 1H), 7.50-7.35 (m, 2H), 4.61 (s, 2H), 3.89 (m, 1H), 3.09 (m, 2H), 2.69 (m, 2H), 1.88 (m, 2H), 1.52 (m, 2H).

¹³C NMR (MeOD-d₄) δ 169.4, 156.1, 143.2, 138.9, 125.8, 123.5, 68.3, 47.9, 45.7, 32.7.

MS (ESI) 236 (M+H⁺).

Using the method described in Example E, the compounds of Examples F and G were similarly prepared from tert-butyl 4-[(chloroacetyl)amino]piperidine-1-carboxylate and the appropriate phenols:

### EXAMPLE F

### N-piperidin-4-yl-2-[3-(trifluoromethoxy)phenoxy]acetamide

Overall yield (two steps) 56%.

¹H NMR (MeOD-d₄) δ 7.33-7.44 (m, 1H), 6.86-7.03 (m, 3H), 4.54 (s, 2H), 3.81-3.95 (m, 1H), 3.01-3.13 (m, 2H), 2.60-2.73 (m, 2H), 1.78-1.92 (m, 2H), 1.40-1.57 (m, 2H).

¹³C NMR (MeOD-d₄) δ 169.7, 160.4, 151.4, 131.8, 121.9 (q, J=255 Hz), 114.9, 114.4, 109.4, 68.4, 48.0, 45.8, 32.9. MS (ESI) 319.2 (M+H⁺).

### EXAMPLE G

### 2-phenoxy-N-piperidin-4-ylacetamide

Overall yield (two steps) 45%

¹H NMR (MeOD-d₄) δ 6.91-7.03 (m, 3H), 7.23-7.34 (m, 2H), 4.48 (s, 2H), 3.81-3.96 (m, 1H), 3.01-3.06 (m, 2H), 2.60-2.69 (m, 2H), 1.82-1.86 (m, 2H), 1.41-1.55 (m, 2H).

¹³C NMR (MeOD-d₄) δ 170.2, 159.2, 130.6, 122.8, 115.8, 68.2, 47.9, 45.8, 32.9.

MS (ESI) 235.3 (M+H⁺).

### EXAMPLE H

### 1-[4-(trifluoromethyl)phenyl]-1H-pyrrole-3-carbaldehyde

To a solution of 2,5-dimethoxy-3-tetrahydrofurancarboxaldehyde (8.0 g, 49.9 mmol) in acetic acid (120 mL) was added 4-aminobenzotrifluoride (8.05 g, 49.9 mmol) and the mixture was heated at reflux under an atmosphere of nitrogen until HPLC indicated that starting material was consumed. The reaction mixture was concentrated and the residue was dissolved in EtOAc (500 mL) and washed with 2M NaOH (aq) (100 mL) and brine. The organic phase was dried (Na₂SO₄) and then evaporated to dryness. The residue was purified on SiO₂ eluted with DCM and finally DCM:MeOH (98:2) to give 8.56 g (72%) of the title compound (94% pure, HPLC purity).

¹H NMR (CDCl₃) δ 9.87 (s, 1H), 7.76 (m, 2H), 7.72 (m, 1H), 7.55 (m, 2H), 7.14 (m, 1H), 6.84 (m, 1H).

¹³C NMR (CDCl₃) δ 185.5, 142.2, 129.4 (q, J=33 Hz), 129.0, 127.4 (q, J=4 Hz), 126.8, 123.8 (q, J=272 Hz), 122.1, 121.1, 110.5.

MS (ESI) 240 (M+H⁺).

Using the method described in Example H, the compounds of Examples I, J, K, L, M, N, and O were similarly prepared from 2,5-dimethoxy-3-tetrahydrofuran-carboxaldehyde and the appropriate aromatic amine:

### EXAMPLE I

### 1-phenyl-1H-pyrrole-3-carbaldehyde

MS (ESI) 272 (M+H⁺).

### EXAMPLE J

### 1-(2-chlorophenyl)-1H-pyrrole-3-carbaldehyde

¹H NMR (DMSO-d₆) δ 9.78 (s, 1H), 7.93 (m, 1H), 7.68-7.74 (m, 1H), 7.50-7.60 (m, 3H), 7.17 (m, 1H), 6.66 (m, 1H).

MS (ESI) 206.2 (M+H⁺).

### EXAMPLE K

### 1-(3-chlorophenyl)-1H-pyrrole-3-carbaldehyde

¹H NMR (CDCl₃) δ 9.85 (s, 1H), 7.65 (m, 1H), 7.45-7.36 (m, 2H), 7.35-7.28 (m, 2H), 7.07 (m, 1H), 6.80 (m, 1H).

¹³C NMR (CDCl₃) δ 185.5, 140.6, 135.7, 131.1, 128.6, 127.5, 127.0, 122.3, 121.5, 119.3, 110.1.

MS (ESI) 206 (M+H⁺).

### EXAMPLE L

### 1-[5-(trifluoromethyl)pyridin-2-yl]-1H-pyrrole-3-carbaldehyde

MS (ESI) 241 (M+H⁺).

### EXAMPLE M

### 1-(4-methoxyphenyl)-1H-pyrrole-3-carbaldehyde

MS (ESI) 202 (M+H⁺).

### EXAMPLE N

### 1-(5-chloropyrimidin-2-yl)-1H-pyrrole-3-carbaldehyde

¹H NMR (CDCl₃) δ 9.90 (s, 1H), 8.63 (s, 2H), 8.36 (m, 1H), 7.76 (m, 1H), 6.78 (m, 1H).

¹³C NMR (CDCl₃) δ 185.8, 157.2, 153.6, 129.5, 128.2, 127.6, 121.6, 110.2.

### EXAMPLE O

### 4-(3-formyl-1H-pyrrol-1-yl)benzonitrile

¹H NMR (CDCl₃) δ 9.88 (s, 1H), 7.80 (d, 2H), 7.73 (m, 1H), 7.55 (d, 2H), 7.15 (m, 1H), 6.86 (m, 1H).

¹³C NMR (CDCl₃) δ 185.4, 134.1, 129.2, 126.6, 124.6, 121.8, 121.1, 118.0, 110.8.

MS (ESI, direct inlet) 197.2 (M+H⁺).

### EXAMPLE P

### 1-(6-trifluoromethyl-pyridin-3-yl)-1H-pyrrole-3-carbaldehyde

To a solution of 2,5-dimethoxy-3-tetrahydrofuranecarboxaldehyde (2.2 g, 13.6 mmol) in acetic acid (40 mL) was added 3-amino-6-(trifluoromethyl)pyridine (2.0 g, 12.3 mmol) and the mixture was heated at 60° C. under an atmosphere of nitrogen until HPLC indicated that the starting material was consumed. The reaction mixture was concentrated and the residue was purified on SiO₂ eluted with heptane:EtOAc (70:30) and finally heptane:EtOAc (60:40). Relevant fractions were combined, concentrated, treated with Et₂O and filtered to give 1.48 g (50%) of the title compound.

¹H NMR (CDCl₃) δ 9.89 (s, 1H), 8.88 (d, 1H), 7.94 (m, 1H), 7.83 (m, 1H), 7.75 (m, 1H), 7.17 (m, 1H), 6.88 (m, 1H). ¹³C NMR (CDCl₃) δ 185.3, 146.7 (q, J=36 Hz), 142.4, 138.0, 129.6, 129.0, 126.5, 122.0, 121.7 (q, J=3 Hz), 121.2 (q, J=274 Hz), 111.3. MS (ESI) 241 (M+H⁺).

### EXAMPLE Q

### 2-(3,4-difluorophenoxy)-N-pyrrolidin-3-ylacetamide

### i) 3-(2-chloro-acetylamino)-pyrrolidine-1-carboxylic acid tert-butyl ester

To a solution of 3-amino-pyrrolidine-1-carboxylic acid tert-butyl ester (4.89 g, 26.3 mmol) and triethylamine (3.19 g, 31.5 mmol) in DCM (50 mL) was added drop wise chloroacetyl chloride (2.30 mL, 28.9 mmol) under an atmosphere of nitrogen. The mixture was stirred at room temperature until LC-MS indicated full conversion of starting material. The solvent was removed and the residue was redissolved in DCM (200 mL). The organic phase was washed with aqueous saturated NaHCO₃ (2×100 mL), brine (100 mL) and dried over Na₂SO₄ before evaporating to dryness. The residue was purified on a SiO₂ column eluting with DCM/MeOH 97:3 to give 2.3 g (33%) of the title compound.

### ii) 3-[2-(3,4-difluoro-phenoxy)-acetylamino]-pyrrolidine-1-carboxylic acid tert-butyl ester

To a solution of 3,4-difluorophenol (0.77 g, 5.9 mmol) in THF (20 mL) was added KO^{t}Bu (0.66 g, 5.9 mmol) and the resulting dark red solution was stirred at room temperature for ca 15 minutes. 3-(2-Chloro-acetylamino)-pyrrolidine-1-carboxylic acid tert-butyl ester (1.42 g, 5.4 mmol) in THF (10 mL) was added and the reaction mixture was heated at 40° C. until judged complete by LC/MS. The mixture was allowed to cool to room temperature, water (50 mL) was added and the mixture was concentrated to ca 50 mL to remove THF. Water (50 mL) and EtOAc (50 mL) was added and the phases were separated. The aqueous phase was extracted with an additional 2×50 mL EtOAc. The combined organic phases were washed with 1M NaOH (50 mL), brine and finally dried over Na₂SO₄. Evaporation of the solvent gave a dark residue that was purified on a SiO₂ column eluting with DCM/MeOH 97:3 to give 1.16 g (60%) of the title compound.

### iii) 2-(3,4-difluorophenoxy)-N-pyrrolidin-3-ylacetamide

To a solution of 3-[2-(3,4-difluoro-phenoxy)-acetylamino]-pyrrolidine-1-carboxylic acid tert-butyl ester (1.16 g, 3.25 mmol) in DCM (30 mL) was added TFA (5 mL) and the mixture was stirred at room temperature for 45 minutes, after which LC-MS indicated full conversion to product. The reaction mixture was evaporated to dryness and the residue was dissolved in EtOAc (200 mL). The organic phase was washed with 2M NaOH (2×50 mL), brine (50 mL) and subsequently dried over Na₂SO₄ and evaporated. The residue was purified on a SiO₂ column eluting first with DCM/MeOH 8:2 and then DCM/MeOH 8:2 containing 2% NH₃ (aq). Relevant fractions were evaporated to dryness, redissolved in CHCl₃ (30 mL) and stirred with 5M NaOH (10 mL) for ca 1 h. The phases were separated and the organic phase was dried over MgSO₄ and evaporated to dryness to give 0.54 g of the title compound as an oil.

¹H NMR (CD₃OD) δ 7.19 (q, 1H), 6.95 (m, 1H), 6.78 (m, 1H), 4.48 (s, 2H), 4.37 (m, 1H), 2.7-3.1 (m, 4H), 2.12 (m, 1H), 1.70 (m, 1H)

MS (ESI⁺) 257.1 (M+H⁺).

### EXAMPLE R

### 1-(2,2-difluoro-benzo[1,3]dioxol-5-yl)-1H-pyrrole-3-carbaldehyde

To a solution of 2,5-dimethoxy-3-tetrahydrofurancarboxaldehyde (1.0 g, 6.4 mmol) in acetic acid (40 ml-L) was added 2,2-difluoro-benzo[1,3]dioxol-5-ylamine (1.0 g, 5.8 mmol) and the mixture was heated at 60° C. under an atmosphere of nitrogen until HPLC indicated that starting material was consumed. The reaction mixture was concentrated and the residue was purified on SiO₂ eluted with heptane:EtOAc (4:1) to give 0.64 g (44%) of the title compound.

¹HNMR (CDCl₃) δ 9.85 (s, 1H), 7.58 (m, 1H), 7.16 (m, 3H), 7.00 (m, 1H), 6.80 (m, 1H).

¹³C NMR (CDCl₃) δ 185.5, 144.6, 142.9, 136.1, 135.4, 132.0, 128.6, 127.4, 122.9, 117.1, 110.3, 110.2, 104.4. MS (ESI) 252 (M+H⁺).

### EXAMPLE S

### 1-(4-trifluoromethanesulfonyl-phenyl)-1H-pyrrole-3-carbaldehyde

To a solution of 2,5-dimethoxy-3-tetrahydrofurancarboxaldehyde (0.78 g, 4.89 mmol) in acetic acid (15 mL) was added 4-(trifluoromethylsulfonyl)aniline (1.0 g, 4.44 mmol) and the mixture was heated at 60° C. under an atmosphere of nitrogen until HPLC indicated that starting material was consumed. The reaction mixture was concentrated and the residue was purified on SiO₂ eluted with heptane:EtOAc (4:1) followed by crystallization from EtOAc/heptane to give 0.52 g (39%) of the title compound.

¹H NMR (DMSO-d₆) δ 9.83 (s, 1H), 8.53 (m, 1H), 8.28-8.16 (m, 4H), 7.79 (m, 1H), 6.78 (m, 1H). MS (ESI) 304 (M+H⁺).

### WORKING EXAMPLES

### Example 1

### 2-(3-chlorophenoxy)-N-{1-[(1-phenyl-1H-pyrrol-3-yl)methylpiperidin-4-yl}acetamide

2-(3-chlorophenoxy)-N-piperidin-4-ylacetamide (0.3 g, 1.1 mmol) and 1-phenyl-1H-pyrrole-3-carbaldehyde (0.2 g, 1.2 mmol) was dissolved in dichloroethane (7 mL). Sodiumtriacetoxyborohydride (0.37 g, 1.75 mmol) was then added and the mixture was stirred at rt. until LC-MS indicated that starting material was consumed. NaHCO₃ (10 mL, aq., sat.) was added, the aqueous phase was extracted with DCM (2×10 mL) and concentrated. The residue was purified on silica gel eluting with DCM:MeOH (95:5) to give 0.1 g (21%) of the title compound.

¹H NMR (MeOD-d₄) δ 7.37-7.50 (m, 4H), 7.18-7.30 (m, 2H), 7.12-15 (m, 2H), 6.97-7.04 (m, 2H), 6.89 (dd, 1H), 6.28 (m, 1H), 4.48 (s, 2H), 3.77 (m, 1H), 3.47 (s, 2H), 2.96 (m, 2H), 2.15 (m, 2H), 1.84 (m, 2H), 1.53-1.68 (m, 2H).

¹³C NMR (MeOD-d₄) δ 169.8, 160.0, 141.8, 136.0, 131.7, 130.7, 126.5, 122.8, 121.8, 120.8, 120.3, 120.2, 116.5, 114.5, 114.3, 113.1, 68.3, 55.9, 52.9, 47.8, 31.9.

MS (ESI) 424 (M+H⁺).

Using the synthetic method described in Example 1, the compounds of Examples 2-6 were similarly prepared from 2-(3-chlorophenoxy)-N-piperidin-4-ylacetamide and the appropriate aldehyde:

### Example 2

### 2-(3-chlorophenoxy)-N-[1-({1-[4-(trifluoromethyl)phenyl]-1H-pyrrol-3-yl}methyl)piperidin-4-yl]acetamide

The residue after workup was purified on silica gel eluted with DCM:MeOH (95:5) to give 0.7 g (83%) of the title compound as a sticky oil. The material was triturated with a mixture of heptane/EtOAc, followed by treatment with Et₂O. Filtration of the solid material afforded 0.160 g of the title compound as a white solid.

¹H NMR (MeOD-d₄) δ 7.81-7.71 (m, 4H), 7.57 (m, 1H), 7.43 (m, 1H), 7.28 (t, 1H), 7.05-6.91 (m, 3H), 6.51 (m, 1H), 4.54 (s, 2H), 4.23 (s, 2H), 4.05 (m, 1H), 3.54 (m, 2H), 3.12 (m, 2H), 2.13 (m, 2H), 1.93 (m, 2H).

¹³C NMR (MeOD-d₄) δ 170.2, 160.0, 143.9, 135.9, 131.7, 128.9 (q, J=33 Hz), 128.1 (q, J=4 Hz), 125.4 (q, J=271 Hz), 123.1, 122.8, 121.7, 121.1, 116.4, 115.4, 114.4, 114.3, 68.2, 54.2, 51.6, 45.4, 29.5.

MS (ESI) 492 (M+H⁺).

### Example 3

### 2-(3-chlorophenoxy)-N-({[1-(4-methoxyphenyl)-1H-pyrrol-3-yl]methyl}piperidin-4-yl)acetamide

The residue after workup was purified on silica gel eluting with DCM:MeOH (95:5). The relevant fractions were concentrated, triturated with Et₂O and subsequently dried to give 0.26 g (48%) of the title compound.

¹H NMR (MeOD-d₄) δ 7.32-7.38 (m, 2H), 7.26 (m, 1H), 6.95-7.06 (m, 6H), 6.91 (dd, 1H), 6.25 (m, 1H), 4.50 (s, 2H), 3.81 (s, 3H), 3.79 (m, 1H), 3.48 (s, 2H), 2.98 (m, 2H), 2.17 (m, 2H), 1.87 (m, 2H), 1.53-1.68 (m, 2H).

¹³C NMR (MeOD-d₄) δ 169.9, 160.0, 159.1, 136.0, 135.6, 131.7, 122.8, 122.5, 121.2, 120.6, 116.5, 115.8, 114.3, 112.6, 68.3, 56.0, 52.9, 47.9, 31.9.

MS (ESI) 454 (M+H⁺).

### Example 4

### 2-(3-chlorophenoxy)-N-(1-{[1-(2-chlorophenyl)-1H-pyrrol-3-yl]methyl}piperidin-4-yl)acetamide

The residue after work-up was purified on silica gel eluting with DCM:MeOH (95:5) to give 0.20 g (64%) of the title compound.

¹H NMR (DMSO-d₆) δ 7.97-7.99 (m, 1H), 7.62-7.64 (m, 1H), 7.37-7.51 (m, 3H), 7.29-7.34 (m, 1H), 7.00-7.04 (m, 2H), 6.89-6.92 (m, 3H), 6.18 (s br, 1H), 4.49 (s, 2H), 3.63 (m, 1H), 3.32 (s, 2H), 2.87 (m, 2H), 1.99 (m, 2H), 1.70 (m, 2H), 1.48-1.55 (m, 2H). MS (ESI) 458 (M+H⁺).

### Example 5

### 2-(3-chlorophenoxy)-N-[1-({1-[5-(trifluoromethyl)pyridin-2-yl]-1H-pyrrol-3-yl}methyl)piperidin-4-yl]acetamide

The residue after work-up was purified on silica gel eluting with first DCM:MeOH (98:2) followed by DCM:MeOH (95:5), concentrated, triturated with Et₂O and subsequently dried to give 0.29 g (63%) of the title compound as an brown solid.

¹H NMR (CDCl₃) δ 8.67 (br s, 1H), 7.95 (m, 1H), 7.45-7.51 (m, 2H), 7.36 (d, 1H), 7.25 (m, 1H), 7.02 (br d, 1H), 6.94 (br s, 1H), 6.81 (m, 1H), 6.34-6.42 (m, 2H), 4.46 (s, 2H), 3.91 (m, 1H), 3.46 (s, 2H), 2.90 (m, 2H), 2.18 (m, 2H), 1.96 (m, 2H), 1.48-1.62 (m, 2H).

¹³C NMR (CDCl₃) δ 166.9, 157.9, 153.3, 146.3 (q, J=4 Hz), 135.9 (q, J=3 Hz), 135.3, 130.7, 123.7 (q, J=271 Hz), 122.7 (q, J=33 Hz), 122.6, 118.6, 117.6, 115.6, 114.0, 112.9, 110.4, 67.6, 55.2, 52.0, 46.3, 32.0.

MS (ESI) 493 (M+H⁺).

### Example 6

### 2-(3-chlorophenoxy)-N-(1-{[1-(3-chlorophenyl)-1H-pyrrol-3-yl]methyl}piperidin-4-yl)acetamide

The residue after work-up was purified on silica gel eluting with first DCM:MeOH (98:2) followed by DCM:MeOH (95:5), concentrated, triturated with Et₂O and subsequently dried to give 0.30 g (70%) of the title compound as an off-white, semisolid material.

¹H NMR (CDCl₃) δ 7.14-7.36 (m, 5H), 6.91-7.02 (m, 4H), 6.79 (m, 1H), 6.42 (br d, 1H), 6.28 (m, 1H), 4.43 (s, 2H), 3.90 (m, 1H), 3.45 (s, 2H), 2.88 (m, 2H), 2.15 (m, 2H), 1.94 (m, 2H), 1.47-1.60 (m, 2H).

¹³C NMR (CDCl₃) δ 166.8, 157.8, 141.5, 135.2, 135.1, 130.5, 125.3, 122.8, 122.3, 120.1, 120.6, 119.0, 118.3, 117.9, 115.5, 112.8, 112.4, 67.4, 55.2, 51.8, 46.2, 32.0. MS (ESI) 458 (M+H⁺).

### Example 7

### 2-(3-chlorophenoxy)-N-[1-(4-pyridin-2-ylbenzyl)piperidin-4-yl]acetamide

2-(3-chlorophenoxy)-N-piperidin-4-ylacetamide (60.0 mg, 0.223 mmol) and 4-pyridin-2-ylbenzaldehyde (49.0 mg, 0.268 mmol) were dissolved in 4 mL of DCM. NaBH(OAc)₃ (85.0 mg, 0.402 mmol) was added and the mixture was stirred at room temperature for about 12 h. A saturated aqueous solution of NH₄Ac (10 mL) was added and the mixture was extracted with EtOAc. The combined organic phase was washed with water, dried over Na₂SO₄ and concentrated. Automated HPLC purification gave the pure title compound as a solid (50 mg, 51%).

¹H NMR (400 MHz, CDCl₃) δ 8.65 (m, 1H), 7.91 (d, 2H), 7.70 (m, 2H), 7.38 (d, 2H), 7.20 (m, 2H), 6.98 (d, 1H), 6.91 (s, 1H), 6.77 (m, 1H), 6.40 (d, 1H), 4.42 (s, 2H), 3.89 (m, 1H), 3.52 (s, 2H), 2.79 (m, 2H), 2.14 (t, 2H), 1.92 (m, 2H), 1.51 (m, 2H).

¹³C NMR (100 MHz, CDCl₃) δ 167.0, 158.0, 157.5, 149.8, 139.6, 138.5, 136.9, 135.4, 130.8, 129.6, 127.0, 122.6, 122.2, 120.6, 115.7, 113.0, 67.7, 62.8, 52.3, 46.5, 32.3.

LC-MS [M+H]⁺ 436.1, [M]⁻ 434.1

Using the synthetic and purification methods described in Example 7, the compounds of Examples 8-13 were similarly prepared from 2-(3-chlorophenoxy)-N-piperidin-4-ylacetamide and the appropriate aldehyde:

### Example 8

### 2-(3-chlorophenoxy)-N-(1-{[5-(4-chlorophenyl)-2-furyl]methyl}piperidin-4-yl)acetamide

¹H NMR (400 MHz, CDCl₃) δ 7.56 (m, 2H), 7.31 (m, 2H), 7.21 (t, 1H), 6.99 (d, 1H), 6.91 (m, 1H), 6.77 (dd, 1H), 6.56 (d, 1H), 6.35 (d, 1H), 6.26 (d, 1H), 4.43 (s, 2H), 3.87 (m, 1H), δ 3.58 (s, 2H), 2.86 (m, 2H), 2.24 (t, 2H), 1.93 (m, 2H), 1.54 (m, 2H).

¹³C NMR (100 MHz, CDCl₃) δ 167.0, 158.0, 152.6, 152.1, 135.4, 133.0, 130.8, 129.6, 129.0, 125.1, 122.6, 115.7, 113.0, 111.2, 106.3, 67.7, 55.1, 51.9, 46.2, 32.2.

LC-MS [M+H]⁺ 459.1; [M]⁻ 457.0.

### Example 9

### 2-(3-chlorophenoxy)-N-[1-({1-[4-(trifluoromethoxy)phenyl]-1H-pyrrol-3-yl}methyl)piperidin-4-yl]acetamide

¹H NMR (400 MHz, CDCl₃) δ 7.36 (m, 2H), 7.26 (m, 2H), 7.21 (t, 1H), 6.96-7.02 (m, 3H), 6.93 (m, 1H), 6.79 (m, 1H), 6.47 (d, 1H), 6.28 (m, 1H), 4.43 (s, 2H), 3.92 (m, 1H), 3.54 (s, 2H), 2.98 (m, 2H), 2.23 (m, 2H), 1.94 (m, 2H), 1.62 (m, 2H).

¹³C NMR (100 MHz, CDCl₃) δ 167.1, 158.0, 146.8, 139.3, 135.4, 130.8, 122.6, 122.0, 122.5, 121.5, 121.3, 119.6, 119.3, 115.7, 113.1, 112.8, 67.6, 54.8, 51.6, 46.2, 31.6.

LC-MS [M+H]⁺ 508.1; [M]⁻ 506.0.

### Example 10

### 2-(3-chlorophenoxy)-N-{1-[3-(1H-pyrrol-1-yl)benzyl]piperidin-4-yl}acetamide

¹H NMR (400 MHz, CDCl₃) δ 7.40-7.15 (m, 5H), 7.11 (m, 2H), 7.02 (m, 1H), 6.95 (t, 1H), 6.81 (m, 1H), 6.38 (bd, 1H), 6.35 (t, 2H), 4.46 (s, 2H), 3.93 (m, 1H), 3.53 (s, 2H), 2.82 (m, 2H), 2.18 (m, 2H), 1.94 (m, 2H), 1.53 (m, 2H).

¹³C NMR (100 MHz, CDCl₃) δ 167.0, 158.0, 141.0, 140.6, 135.5, 130.8, 129.6, 126.3, 122.7, 121.0, 119.5, 119.4, 115.8, 113.0, 110.6, 67.7, 62.9, 52.4, 46.5, 32.4.

LC-MS [M+H]⁺ 424.2; [M]⁻ 422.1

### Example 11

### 2-(3-chlorophenoxy)-N-[1-(3-pyridin-2-ylbenzyl)piperidin-4-yl]acetamide

¹H NMR (400 MHz, CDCl₃) δ 8.67 (m, 1H), 7.93 (s, 1H), 7.85 (m, 1H), 7.73 (m, 2H), 7.40 (m, 2H), 7.22 (m, 2H), 7.00 (m, 1H), 6.92 (t, 1H), 6.78 (dd, 1H), 6.39 (d, 1H), 4.43 (s, 2H), 3.91 (m, 1H), 3.57 (s, 2H), 2.83 (m, 2H), 2.18 (m, 2H), 1.92 (m, 2H), 1.52 (m, 2H).

¹³C NMR (100 MHz, CDCl₃) δ 167.0, 158.0, 157.7, 149.9, 139.6, 139.2, 136.9, 135.5, 130.8, 129.9, 128.9, 127.9, 126.0, 122.6, 122.3, 120.9, 115.7, 113.0, 67.9, 63.2, 52.3, 46.5, 32.3.

LC-MS [M+H]⁺ 436.2; [M]⁻ 434.1

### Example 12

### 2-(3-chlorophenoxy)-N-(1-{[5-(2,4-dichlorophenyl)-2-furyl]methyl}piperidin-4-yl)acetamide

¹H NMR (400 MHz, CDCl₃) δ 7.77 (d, 1H), 7.43 (d, 1H), 7.27 (dd, 1H), 7.22 (d, 1H), 7.05 (d, 1H), 7.00 (m, 1H), 6.92 (m, 1H), 6.78 (dd, 1H), 6.35 (d, 1H), 6.32 (d, 1H), 4.44 (s, 2H), 3.88 (m, 1H), 3.61 (s, 2H), 2.87 (m, 2H), 2.25 (m, 2H), 1.95 (m, 2H), 1.54 (m, 2H).

¹³C NMR (100 MHz, CDCl₃) δ 167.1, 158.0, 152.2, 149.0, 135.5, 133.0, 130.8, 130.6, 128.8, 128.0, 127.4, 122.7, 115.7, 113.0, 112.2, 111.2, 67.7, 55.0, 52.0, 46.2, 32.2.

LC-MS [M+H]⁺ 495.0; [M]⁻ 492.9.

### Example 13

### 2-(3-chlorophenoxy)-N-[1-({5-[1-methyl-5-(trifluoromethyl)-1H-pyrazol-3-yl]-2-thienyl}methyl)piperidin-4-yl]acetamide

¹H NMR (400 MHz, CDCl₃) δ 7.24 (dd, 1H), 7.01 (m, 2H), 6.92 (m, 2H), 6.80 (dd, 1H), 6.59 (s, 1H), 6.39 (d, 1H), 4.45 (s, 2H), 4.00 (s, 3H), 3.92 (m, 1H), 3.71 (s, 2H), 2.89 (m, 2H), 2.22 (m, 2H), 1.93 (m, 2H), 1.54 (m, 2H).

¹³C NMR (100 MHz, CDCl₃) δ 167.1, 158.0, 145.6, 138.8, 135.5, 130.8, 129.1, 127.5, 126.3, 122.7, 115.7, 113.0, 104.8, 67.7, 57.3, 52.3, 46.3, 38.7, 32-3

LC-MS [M+H]⁺ 513.1; [M]⁻ 511.0.

### Example 14

### 2-(3-chlorophenoxy)-N-(1-{[1-(4-bromophenyl)-1H-pyrrol-3-yl]methyl}piperidin-4-yl)acetamide

2-(3-chlorophenoxy)-N-piperidin-4-ylacetamide (53.7 mg, 0.200 mmol) was dissolved in MeOH (0.67 ml), 1-(4-bromophenyl)-1H-pyrrole-3-carbaldehyde (75 mg, 0.300 mmol) dissolved in DCM (3 ml) and acetic acid (0.1 ml) was added to a process vial charged with polymer-supported cyanoborohydride (93 mg, 4.3 mmol/g, Nova Biochem). The mixture was heated to 140° C. for 15 minutes in a microwave oven. After filtration PS-Isocyanate (50 mg, 0.07 mmol, Argonaut) and PS-Trisamine (50 mg, 0.22 mmol, Argonaut) was added to scavenge unreacted material. Filtration, evaporation automated HPLC purification gave the title compound.

¹H NMR (400 MHz, CDCl₃) δ 7.52 (m, 2H), 7.24 (m, 3H), 6.92-7.04 (m, 4H), 6.79 (dd, 1H), 6.34 (m, 1H); 6.27 (dd, 1H), 4.44 (s, 2H), 3.90 (m, 1H), 3.44 (s, 2H), 2.89 (d, 2H), 2.15 (dd, 2H), 1.94 (d, 2H), 1.52 (m, 2H).

LC-MS [M+H]⁺ 502.5, 504.5; [M]⁻ 500.8, 502.8

### Example 15

### 2-(3-chlorophenoxy)-N-methyl-N-[1-({1-[4-(trifluoromethyl)phenyl]-1H-pyrrol-3-yl}methyl)piperidin-4-yl]acetamide

### i) N-(1-benzylpiperidin-4-yl)-2-chloro-N-methylacetamide

Chloroacetyl chloride (1.1 mL, 14 mmol) was added dropwise to a stirred solution of 1-benzyl-N-methylpiperidin-4-amine (2.5 g, 12 mmol, prepared as described by Russell, M. G. N. et al. J. Med. Chem., 1999, 42, 4981) in DCM (50 mL) at 0° C. The mixture was stirred for 1 h at rt. whereupon additional DCM (100 mL) was added and the organic phase was washed with NaHCO₃ (50 mL, aq., sat.), dried over MgSO₄ and concentrated to give 3.4 g (quant.) of the title compound as a thick slightly yellow oil which was used in the next step without further purification.

¹H NMR (DMSO-d₆, complex rotameric mixture, * denotes minor rotamer peaks) δ 7.20-7.38 (m, 5H), 4.40* (s, 2H), 4.35 (s, 2H), 4.18 (m, 1H), 3.58* (m, 1H), 3.48* (s, 2H), 3.47 (s, 2H), 2.88 (br s, 1H), 2.84 (s, 3H), 2.72* (s, 3H), 1.95-2.12 (m, 2H), 1.56-1.84 (m, 3H), 1.43 (m, 2H).

MS (ESI) 281.3 (M+H⁺).

### ii) N-(1-benzylpiperidin-4-yl)-2-(3-chlorophenoxy)-N-methylacetamide

Potassium tert-butoxide (1.05 g, 9.3 mmol) was added portionwise to a solution of 3-chlorophenol (1.2 g, 9.3 mmol) in THF (15 mL) and the mixture was stirred until a clear solution was obtained. N-(1-benzylpiperidin-4-yl)-2-chloro-N-methylacetamide (1.5 g, 5.3 mmol) dissolved in THF (15 mL) was added dropwise and the mixture was stirred for 1.5 h. Water (10 mL) was added and the mixture was extracted with EtOAc (2×50 mL) and the combined organic phases were washed with 1 M NaOH (2×20 mL). The organic phase was concentrated and the residue was purified on silica gel eluted with DCM:MeOH (95:5) to give 2.0 g (quant) of the title compound as a off-white solid.

¹H NMR (DMSO-d₆, complex rotameric mixture, * denotes minor rotamer peaks) δ 7.20-7.36 (m, 6H), 6.95-7.03 (m, 2H), 6.87 (br d, 1H), 4.90* (s, 2H), 4.84 (s, 2H), 4.19 (m, 1H), 3.55* (m, 1H), 3.47* (s, 2H), 3.45 (s, 2H), 2.87 (br s, 1H), 2.84 (s, 3H), 2.72* (s, 3H), 1.93-2.08 (m, 2H), 1.58-1.84 (m, 3H), 1.42 (m, 2H).

MS (ESI) 373.3 (M+H⁺).

### iii) 2-(3-chlorophenoxy)-N-methyl-N-piperidin-4-ylacetamide

1-Chloroethyl chloroformate (1.2 g, 8.4 mmol) was added to a solution of N-(1-benzylpiperidin-4-yl)-2-(3-chlorophenoxy)-N-methylacetamide (4.1 g, 11.4 mmol) in dichloroethane (30 mL) and the mixture was heated at reflux for 2.5 h. The reaction mixture was concentrated and methanol (30 mL) was added and heated to reflux until for 1 h (over night). The reaction mixture was concentrated and the residue was dissolved in HCl diluted with water (50 mL) and extracted with Et₂O (2×25 mL). The aqueous phase was made basic with NaOH and extracted with EtOAc (2×50 mL). The combined organic phases were concentrated and the residue was purified on silica gel eluted with first DCM:MeDH (9:1) followed by DCM:MeOH (8:2) containing 1% NH₃ (aq.) and finally with DCM:MeOH (7:3) containing 1% TEA, to give 0.82 g (65%) of the title compound after drying.

¹H NMR (MeOD-d₄, complex rotameric mixture, * denotes minor rotamer peaks) δ 7.21-7.29 (m, 1H), 6.86-7.03 (m, 3H), 4.86* (s, 2H), 4.81 (s, 2H), 4.45 (m, 1H), 3.81* (m, 1H), 3.11 (m, 2H), 2.96 (s, 3H), 2.86* (s, 3H), 2.61-2.73 (m, 2H), 1.56-1.86 (m, 4H). MS (ESI) 283.2 (M+H⁺).

### iv) 2-(3-chlorophenoxy)-N-methyl-N-[1-({1-[4-(trifluoromethyl)phenyl]-1H-pyrrol-3-yl}methyl)piperidin-4-yl]acetamide

2-(3-chlorophenoxy)-N-methyl-N-piperidin-4-ylacetamide (0.40 g, 1.4 mmol) and 1-[4-(trifluoromethyl)phenyl]-1H-pyrrole-3-carbaldehyde (0.34 g, 1.4 mmol) was dissolved in dichloroethane (20 mL). Sodium triacetoxyborohydride (0.42 g, 1.4 mmol) was added and the mixture was stirred at rt. for 16 h (over night). NaHCO₃ (10 mL, aq., sat.) was added and the aqueous phase was extracted with DCM (2×20 mL). The combined organic phases were concentrated and the residue was purified on silica gel eluting with DCM:MeOH (98:2) followed by DCM:MeOH (95:5) to give 0.56 g (79%) of the title compound as a off-white solid.

¹H NMR (MeOD-d₄, complex rotameric mixture, * denotes minor rotamer peaks) δ 7.73 (d, 2H), 7.66 (d, 2H), 7.20-7.31 (m, 3H), 6.93-7.00 (m, 2H), 6.86-6.91 (m, 1H), 6.36 (br s, 1H), 4.84* (s, 2H), 4.80 (s, 2H), 4.37 (m, 1H), 3.71* (m, 1H), 3.51 (s, 2H), 3.10 (m, 2H), 2.95 (s, 3H), 2.86* (s, 3H), 2.10-2.24 (m, 2H), 1.58-2.03 (m, 4H).

¹³C NMR (MeOD-d₄, complex rotameric mixture, * denotes minor rotamer peaks) δ 169.9*, 169.8, 160.5, 160.0*, 144.5, 136.0*, 135.9, 131.6*, 131.5, 128.0 (q, J=4 Hz), 128.0 (q, J=33 Hz), 125.8 (q, J=271 Hz), 123.2, 122.6*, 122.5, 120.4, 120.3, 120.1, 120.0*, 116.3, 116.1*, 114.4, 114.1*, 68.1*, 67.7, 55.7, 55.6*, 53.4, 53.2, 52.9, 30.1, 29.1, 28.9, 28.1*.

MS (ESI) 506.3 (M+H⁺).

### Example 16

### 2-[(3-chlorophenyl)thio]-N-[1-({1-[4-(trifluoromethyl)phenyl]-1H-pyrrol-3-yl}methyl)piperidin-4-yl]acetamide

### i) N-(1-benzylpiperidin-4-yl)-2-[(3-chlorophenyl)thio]acetamide

Potassium tert-butoxide (1.26 g, 11.3 mmol) was added portionwise to a solution of 3-chlorothiophenol (1.8 g, 12.4 mmol) in THF (20 mL) and the mixture was stirred until a clear solution was obtained. N-(1-benzylpiperidin-4-yl)-2-chloroacetamide (3 g, 11.3 mmol) dissolved in THF (25 mL) was added dropwise and the mixture was stirred over night at rt. HPLC indicated that starting material was consumed The solvent was removed by evaporation and the residue was purified on silica gel eluted with DCM:MeOH (95:5) to give 2.35 g (57%) of the title compound.

¹H NMR (CDCl₃) δ 7.10-7.29 (m, 9H), 6.58 (d br, 1H), 3.77 (m, 1H), 3.59 (s, 2H), 3.43 (s, 2H), 2.66 (m, 2H), 2.08 (m, 2H), 1.77 (m, 2H), 1.38 (m, 2H).

MS (ESI) 375.2 (M+H⁺).

### ii) 2-[(3-chlorophenyl)thio]-N-piperidin-4-ylacetamide

1-Chloroethyl chloroformate (1.1 g, 6.7 mmol) was added to a solution of N-(1-benzylpiperidin-4-yl)-2-[(3-chlorophenyl)thio]acetamide (1.9 g, 5.1 mmol) in dichloroethane (30 mL) and the mixture was stirred first at rt. for 1 h and then heated at reflux for 1 h. The reaction mixture was concentrated and methanol (30 mL) was added and heated to reflux for 1 h and then stirred at rt. over night. The reaction mixture was concentrated and the residue was dissolved in toluene and evaporated to dryness. The resulting residue was diluted with DCM and washed with 5 M NaOH (aq.). The organic layer was separated and concentrated and the residue was purified on silica gel eluted with first DCM:MeOH (8:2) followed by DCM:MeOH (8:2) containing 0.5% NH₃ (25% aq.) and then pure MeOH to give 0.30 g (21%) of the title compound.

¹H NMR (CDCl₃) δ 7.09-7.30 (m, 4H), 6.58 (br d, 1H), 3.86 (m, 1H), 3.60 (s, 2H), 2.98 (m, 2H), 2.55-2.76 (m, 2H), 1.81 (m, 2H), 1.64 (br s, 1H) 1.14-1.34 (m, 2H).

MS (ESI) 286.2 (M+H⁺).

### iii) 2-[(3-chlorophenyl)thio]-N-[1-({1-[4-(trifluoromethyl)phenyl]-1H-pyrrol-3-yl}methyl)piperidin-4-yl]acetamide

2-[(3-Chlorophenyl)thio]-N-piperidin-4-ylacetamide (0.30 g, 1.1 mmol) and 1-[4-(trifluoromethyl)phenyl]-1H-pyrrole-3-carbaldehyde (0.25 g, 1.1 mmol) was dissolved in dichloroethane (7 mL). Sodium triacetoxyborohydride (0.31 g, 1.5 mmol) was added and the mixture was stirred at rt. for 3 h and 45 min. Sat. aq. NaHCO₃ (11 mL) was added and the aqueous phase was extracted with DCM. The organic layer was separated and dried over Mg₂SO₄ and concentrated. The residue was purified on silica gel eluting with DCM:MeOH (95:5) to give 0.25 g (47%) of the title compound.

¹H NMR (CDCl₃) δ 7.61-7.72 (m, 2H), 7.41-7.51 (m, 2H), 7.10-7.30 (m, 4H), 6.99-7.10 (m, 2H), 6.58-6.61 (br d, 1H), 6.31 (m, 1H), 3.72-3.89 (m, 1H), 3.61 (s, 2H), 3.45 (s, 2H), 2.75-2.91 (m, 2H), 2.09-2.27 (m, 2H), 1.76-1.92 (m, 2H), 1.36-1.59 (m, 2H)

¹³C NMR (CDCl₃) δ 143.1, 136.7, 135.2, 130.4, 128.0, 127.4 (q, J=33 Hz), 127.0 (q, J=3 Hz), 127.0, 126.2, 124.1 (q, J=271 Hz), 122.7, 119.7, 119.2, 118.6, 113.1, 55.2, 51.8, 46.7, 37.3, 31.7.

MS (ESI) 508.2 (M+H⁺).

### Example 17

### 2-(3-chlorophenoxy)-N-(1-{[1-(4-cyanophenyl)-1H-pyrrol-3-yl]methyl}piperidin-4-yl)acetamide

2-(3-chlorophenoxy)-N-piperidin-4-ylacetamide (1 eq, 0.279 mmol) and 4-(3-formyl-1H-pyrrol-1-yl)benzonitrile (1.2 eq) were dissolved in DCM (5 ml) and left to stir for 5-10 minutes. MP-BH(OAc)₃ (2.5 meq) was added and the reaction stirred for a further 3 h at ambient temperature. The reaction was filtered, washed through with DCM (2 ml) and the filtrate concentrated in vacuo. Flash silica chromatography on a 9 g or 40 g Biotage cartridge eluting with EtOAc/MeOHITEA (100/2/0.2) yielded the product as a white foam (81 mg, 65%).

¹HNMR (CDCl₃) δ 7.67 (d, 2H), 7.43 (d, 2H), 7.22 (t, 1H), 6.77-7.08 (m, 15H), 6.32-6.38 (m, 2H), 4.43 (s, 2H), 3.89 (m, 1H), 3.42 (s, 2H), 2.86 (d, 2H), 2.13 (t, 2H>, 1.92 (m, 2H), 1.50 (m, 2H).

¹³CNMR (CDCl₃): δ 167.0, 158.0, 143.7, 135.4, 134.0, 130.8, 124.5, 122.6, 119.7, 119.0, 118.7, 118.1, 115.8, 113.8, 113.0, 108.5, 67.7, 55.4, 52.2, 46.5, 32.3.

MS (ESI): 449.3 (M+H⁺)

This method used in the preparation of the compound of Example 17, with minor variations, was used on a 0.1-1 mmol scale for the synthesis of the compounds of Examples 18-26.

### Example 18

### 2-(pyridin-3-yloxy)-N-[1-({1-[4-(trifluoromethyl)phenyl]-1H-pyrrol-3-yl}methyl)piperidin-4-yl]acetamide

¹HNMR (CDCl₃) δ 8.32 (d, 1 μl), 8.26 (dd, 1H), 7.62 (d, 2H), 7.42 (d, 2H), 7.16-7.26 (m, 2H), 7.02 (m, 2H) 6.43 (d, 1H), 6.29 (m, 1H), 4.47 (s, 2H), 3.85-3.92 (m, 1H), 3.42 (s, 2H), 2.87 (d, 2H), 2.12 (t, 2H), 1.93 (d, 2H), 1.47-1.57 (m, 2H).

MS (ESI): 459.2 (M+H⁺)

### Example 19

### 2-[3-(trifluoromethoxy)phenoxy]-N-[1-({1-[4-(trifluoromethyl)phenyl]-1H-pyrrol-3-yl}methyl)piperidin-4-yl]acetamide

¹HNMR (CDCl₃) δ 7.63 (d, 2H), 7.43 (d, 2H) 7.30 (t, 1H), 7.04 (m, 2H), 6.78-6.89 (m, 3H), 6.38 (d, 1H), 6.30 (m, 1H), 4.45 (s, 2H) 3.85-3.94 (m, 1H), 3.43 (s, 2H), 2.87 (d, 2H), 2.14 (t, 2H) 1.93 (d, 2H), 1.48-1.57 (m, 2H).

¹³CNMR (CDCl₃): δ 166.8, 158.3, 150.4, 143.2; 130.8, 126 (q, 257), 12.4 (q, J=33.8), 127.1 (q, J=3.4), 124.4 (q, J=270), 123.8, 119.6, 119.3, 118.3, 114.6, 113.1, 113.0, 108.4, 67.8, 55.4, 52.2, 46.5, 32.3

MS (ESI): 542.4 (M+H⁺)

### Example 20

### 2-[3-(trifluoromethoxy)phenoxy]-N-[1-({1-[5-(trifluoromethyl)pyridin-2-yl]-1H-pyrrol-3-yl}methyl)piperidin-4-yl]acetamide

¹HNMR (CDCl₃) δ 8.62 (s, 1H), 7.90 (dd, 1H), 7.47 (t, 1H), 7.42 (s, 1H), 7.32-7.29 (m, 2H) 6.77-6.88 (m, 3H), 6.37 (d, 1H) 6.32 (m, 1H) 4.44 (s, 2H), 3.85-3.92 (m, 1H), 3.42 (s, 2H), 2.85 (d, 2H), 2.14 (t, 2H), 1.92 (d, 2H), 1.47-1.56 (m, 2H).

MS (ESI): 543.4 (M+H⁺)

### Example 21

### 2-(3-cyanophenoxy)-N-[1-({1-[4-(trifluoromethyl)phenyl]-1H-pyrrol-3-yl}methyl)piperidin-4-yl]acetamide

¹HNMR (CDCl₃) δ 7.65 (d, 2H), 7.45 (m, 3H), 7.35 (d, 1H), 7.22 (m, 2H), 7.05 (d, 2H), 6.35 (m, 2H), 4.44 (s, 2H), 3.86-3.98 (m, 1H), 3.64 (s, 2H), 2.90 (d, 2H), 2.18 (t, 2H), 1.95 (m, 2H), 1.55 (m, 2H).

¹³CNMR (CDCl₃): δ 166.4, 157.4, 143.2, 131.0, 127.4 (q, J=32.7), 127.1 (q, J=3.6), 126.1, 124.3 (q, J=271), 123.6, 119.7, 119.6, 119.2, 118.5, 118.4, 113.9, 113.1, 67.7, 55.4, 52.2, 46.6, 32.3.

MS (ESI): 483.2 (M+H⁺)

### Example 22

### 2-(3-fluorophenoxy)-N-[1-({1-[4-(trifluoromethyl)phenyl]-1H-pyrrol-3-yl}methyl)piperidin-4-yl]acetamide

¹HNMR (CDCl₃) δ 7.65 (d, 2H), 7.45 (d, 2H), 7.25 (dd, 1H), 7.05 (d, 2H), 6.6-6.8 (m, 3H), 6.4 (br.d, 1H), 6.30 (s, 1H), 4.44 (s, 2H), 3.84-3.93 (m, 1H), 3.64 (s, 2H), 2.90 (d, 2H), 2.15 (t, 2H), 1.95 (m, 2H), 1.55 (m, 2H).

¹³CNMR (CDCl₃): δ 167.1, 163.8 (d, J=246), 158.6 (d, J=11.3), 143.2, 130.8 (d, J=10.1), 127.4 (q, J=33.8), 127.1 (q, J=3.4), 124.4 (q, J=270), 123.7, 119.7, 119.2, 118.4, 113.1, 110.4, 109.3 (d, J=22.7), 103.1 (d, J=22.7), 67.7, 55.4, 52.1, 46.5, 32.3.

MS (ESI): 476.2 (M+H⁺)

### Example 23

### 2-(3-cyanophenoxy)-N-[1-({5-[1-methyl-5-(trifluoromethyl)-1H-pyrazol-3-yl]-2-thienyl}methyl)piperidin-4-yl]acetamide

¹HNMR (CDCl₃) δ 7.42 (t, 1H), 7.32 (d, 1H), 7.15 (m, 2H), 7.05 (d, 1H), 6.9 (d, 1H), 6.6 (s, 1H), 6.38 (br.d, 1H), 4.48 (s, 2H), 4.0 (s, 3H), 3.90-3.98 (m, 1H), 3.70 (s, 2H), 2.90 (d, 2H), 2.25 (t, 2H), 1.95 (m, 2H), 1.55 (m, 2H).

MS (ESI): 504.2 (M+H⁺)

### Example 24

### 2-(2-chlorophenoxy)-N-[1-({1-[4-(trifluoromethyl)phenyl]-1H-pyrrol-3-yl}methyl)piperidin-4-yl]acetamide

¹HNMR (CDCl₃) δ 7.65 (d, 2H), 7.45 (d, 2H), 7.40 (d, 1H), 7.25 (t, 1H), 7.05 (d, 2H), 6.95 (t, 1H), 6.8-6.9 (m, 2H), 6.35 (s, 1H), 4.48 (s, 2H), 3.84-3.93 (m, 1H), 3.62 (s, 2H), 2.78 (d, 2H), 2.25 (t, 2H), 1.95 (m, 2H), 1.60 (m, 2H).

¹³CNMR (CDCl₃): δ 166.9, 153.0, 143.2, 130.6, 128.3, 127.4 (q, J=33.8), 127.1 (q, J=3.6), 124.3 (q, J=271), 123.7, 123.1, 123.0, 119.7, 119.2, 118.4, 114.1, 113.2, 68.2, 55.5, 52.0, 46.1, 32.3

MS (ESI): 492.3 (M+H⁺)

### Example 25

### 2-(3-chlorophenoxy)-N-[1-({5-[4-(trifluoromethoxy)phenyl]-2-furyl}methyl)piperidin-4-yl]acetamide

¹HNMR (CDCl₃) δ 7.65 (d, 2H), 7.21 (m, 3H), 7.00 (m, 1H), 6.92 (t, 1H), 6.78 (dd, 1H), 6.57 (d, 1H), 6.35 (d, 1H), 6.27 (d, 1H), 4.43 (s, 2H), 3.84-3.92 (m, 1H), 3.59 (s, 2H), 2.86 (d, 2H), 2.24 (t, 2H), 1.94 (d, 2H), 1.49-1.58 (m, 2H).

¹³CNMR (CDCl₃): δ 167.0, 158.0, 152.4, 152.3, 148.3, 135.5, 130.8, 129.9, 125.2, 122.6, 121.4, 120.6 (q, J=258), 115.7, 113.0, 111.2, 106.5, 67.7, 55.1, 51.9, 46.2, 32.2

MS (ESI): 509.2 (M+H⁺)

### Example 26

### 2-(3-chlorophenoxy)-N-(1-{[1-(5-chloropyrimidin-2-yl)-1H-pyrrol-3-yl]methyl}piperidin-4-yl)acetamide

¹HNMR (CDCl₃) δ 8.5 (s, 1H), 7.63 (m, 1H), 7.55 (m, 1H), 7.2 (dd, 1H), 6.95 (d, 1H), 6.90 (t, 1H), 6.77 (dd, 1H), 6.36 (br.d, 1H), 6.27 (m, 1H), 4.42 (s, 2H), 3.84-3.93 (m, 1H), 3.4 (s, 2H), 2.85 (d, 2H), 2.15 (t, 2H), 1.95 (m, 2H), 1.50 (m, 2H).

MS (ESI): 460.1 (M+H⁺)

### Example 27

### 2-(3-cyanophenoxy)-N-[1-({1-[4-(trifluoromethoxy)phenyl]-1H-pyrrol-3-yl}methyl)piperidin-4-yl]acetamide

2-(3-cyanophenoxy)-N-piperidin-4-ylacetamide (1 eq, 1.93 mmol) and 1-[4-(trifluoromethoxy)phenyl]-1H-pyrrole-3-carbaldehyde (1.2 eq) were dissolved in DCM (3 ml) and stirred for 10 minutes. NaBH(OAc)₃ (2.5 eq) was then added and the reaction stirred for 16 h. To the reaction mixture was added 10% Na₂CO₃ (aq) (3 ml) shaken and filtered over a phase separator onto a 1 g SCX-2 column. The phase separation was washed through with DCM (1 ml) and the SCX-2 with DCM (5 ml). The product was released from the cation exchanger with 2M NH3 in MeOH (2.5 ml) the filtrate collected and evaporated in vacuo. Flash chromatography on the Biotage 9 g silica cartridge using isocratic EtOAc:MeOH:TEA (100:5:0.1) gave product in unsatisfactory purity. The compound was further purified by automated HPLC purification to yield the compound as its mono acetate salt (23 mg, 21%).

¹HNMR (CDCl₃) 8.7.70 (d, 1H), 7.42 (m, 2H), 7.33 (m, 1H), 7.28 (dd, 1H), 7.13-7.20 (m, 2H), 7.05 (d, 1H), 6.32 (m, 2H), 4.48 (s, 2H), 3.84-3.93 (m, 1H), 3.62 (s, 2H), 2.89 (d, 2H), 2.25 (t, 2H), 1.95 (m, 2H), 1.50-1.60 (m, 2H).

MS (ESI): 499.3 (M+H⁺)

This method was also used for the synthesis of the compound of Example 28:

### Example 28

### 2-(3-cyanophenoxy)-N-({[5-(2,4-dichlorophenyl)-2-furyl]methyl}piperidin-4-yl)acetamide

¹HNMR (CDCl₃) δ 7.78 (d, 1H), 7.40-45 (m, 2H), 7.26-7.34 (m, 2H), 7.13-7.20 (m, 2H), 7.05 (d, 1H), 6.28-6.36 (m, 2H), 4.44 (s, 2H), 3.84-3.93 (m, 1H), 3.62 (s, 2H), 2.85 (d, 2H), 2.25 (t, 2H), 1.95 (m, 2H), 1.50-1.60 (m, 2H).

MS (ESI): 484.0 (M+H⁺)

### Example 29

### 3-(3-chlorophenyl)-N-[1-({1-[4-(trifluoromethyl)phenyl]-1H-pyrrol-3-yl}methyl)piperidin-4-yl]propanamide

### i) tert-butyl-[1-({1-[4-(trifluoromethyl)phenyl]-1H-pyrrol-3-yl}methyl)-piperidin-4-yl]-carbamate

1-[4-(trifluoromethyl)phenyl]-1H-pyrrole-3-carbaldehyde (4.054 g, 16.95 mmol) and tert-butyl piperidin-4-ylcarbamate, (3.564 g, 17.80 mmol) was suspended in DCM (35 mL). Sodium triacetoxyborohydride (7.184 g, 33.90 mmol) was added and stirred overnight at rt. The reaction mixture was quenched with sat. aq. NH₄Cl solution (30 mL), extracted with DCM (3×40 mL), washed with brine (30 mL), dried with Na₂SO₄ and purified with Biotage Horizon Pioneer® HPFS using a silica cartridge and eluted with EtOAc:MeOH:TEA (gradient from 100:0:0 to 100:5:0.1) to give 6.12 g (85%) of the title compound as a white solid.

¹HNMR (MeOD-d₄) δ 7.77 (d, 2H), 7.71 (d, 2H), 7.51 (s, 1H), 7.40 (t, 1H) 6.48 (m, 1H), 4.08 (s, 2H), 3.55-3.58 (m, 1H), 3.38 (d, 2H), 2.84 (t, 2H), 2.08 (m, 2H), 1.72 (m, 2H), 1.43 (s, 9H).

MS (ESI) 424.3 (M+1H⁺).

### ii) 1-({1-[4-(trifluoromethyl)phenyl]-1H-pyrrol-3-yl}methyl)piperidin-4-amine dihydrochloride

tert-butyl-[1-({1-[4-(trifluoromethyl)phenyl]-1H-pyrrol-3-yl}methyl)piperidin-4-yl]carbamate (6.119 g, 14.45 mmol) was dissolved in HCl 4 M in 1,4-dioxane (35 mL) and stirred at rt. for 1.5 hours. Diethyl ether (10 mL) was added to the suspension which was stirred for 1.5 hours. The precipitate was filtered off and was washed with diethyl ether (200 mL) and was then dried at reduced pressure over night to give 4.98 g (87%) of the title compound as a cream-coloured white solid.

¹HNMR (MeOD-d₄) δ 7.77 (m, 4H), 7.63 (s, 1H), 7.40 (t, 1H), 6.56 (s, 1H), 4.28 (s, 2H), 3.65-3.69 (m, 2H), 3.49 (m, 1H), 3.16 (t, 2H), 2.30 (m, 2H), 1.99-2.10 (m, 2H).

MS (ESI) 325.2 (M+1H⁺).

### iii) 3-(3-chlorophenyl)-N-[1-({1-[4-(trifluoromethyl)phenyl]-1H-pyrrol-3-yl}methyl)piperidin-4-yl]propanamide

1-({1-[4-(trifluoromethyl)phenyl]-1H-pyrrol-3-yl}methyl)piperidin-4-amine dihydrochloride (0.050 g, 0.126 mmol) and 3-(3-chlorophenyl)propionic acid (0.028 g, 0.152 mmol) was dissolved in DMF (7 mL). DIPEA (0.077 mL, 0.445 mmol) was added followed by HATU (0.058 g, 0.153 mmol). The mixture was stirred for 3 hours at room temperature. EtOAc (10 mL) was added and the reaction mixture was washed with 1% Na₂CO₃ aq. solution (3×10 mL), dried (MgSO₄), concentrated and purified with Biotage Horizon Pioneer® HPFS using a silica cartridge and eluted with EtOAc:MeOH:TEA (100:5:0.1) to give the title compound (51 mg, 83%).

¹HNMR (MeOD-d₄) δ 7.70 (d, 2H), 7.62 (d, 2H), 7.19-7.26 (m, 4H), 7.09-7.16 (m, 2H), 6.33 (bs, 1H), 3.57-3.65 (m, 1H), 3.45 (s, 2H), 2.85-2.91 (m, 4H), 2.43 (t, 2H), 2.12 (t, 2H), 1.76 (d, 2H), 1.38-1.47 (m, 2H).

MS (ESI) 490.2 (M+1H⁺).

### Example 30

### (2E)-3-(3-chlorophenyl)-N-[1-({1-[4-(trifluoromethyl)phenyl]-1H-pyrrol-3-yl}methyl)piperidin-4-yl]acrylamide

1-({1-[4-(trifluoromethyl)phenyl]-1H-pyrrol-3-yl}methyl)piperidin-4-amine dihydrochloride (0.050 g, 0.126 mmol) and (2E)-3-(3-chlorophenyl)acrylic acid (0.028 g, 0.153) was dissolved in DMF (7 mL). DIPEA (0.077 mL, 0.445 mmol) was added followed by HATU (0.057 g, 0.153 mmol). The mixture was stirred for 3 hours at room temperature. EtOAc (10 mL) was added and the mixture was washed with 1% Na₂CO₃ aq. solution (3×10 mL), dried (MgSO₄), concentrated and purified with Biotage Horizon Pioneer® HPFS using a silica cartridge and eluted with EtOAc:MeOH:TEA (100:5:0.1) to give the title compound (55 mg, 89%) as a solid.

¹HNMR (MeOD-d₄) δ 7.70 (d, 2H), 7.62 (d, 2H), 7.53 (s, 1H), 7.41-7.47 (m, 2H), 7.32-7.33 (m, 2H), 7.25 (m, 2H), 6.59 (d, 1H), 6.34 (t, 1H), 3.74-3.82 (m, 1H), 3.49 (s, 2H), 2.99 (d, 2H), 2.19 (t, 2H), 1.91 (m, 2H), 1.54-1.64 (m, 2H).

MS (ESI) 488.1 (M+H⁺).

### Example 31

### 2-(3,5-difluorophenoxy)-N-[1-({1-[4-(trifluoromethyl)phenyl]-1H-pyrrol-3-yl}methyl)piperidin-4-yl]acetamide

### i) 2-chloro-N-[1-({1-[4-(trifluoromethyl)phenyl]-1H-pyrrol-3-yl}methyl)-piperidin-4-yl]acetamide

1-({1-[4-(trifluoromethyl)phenyl]-1H-pyrrol-3-yl}methyl)piperidin-4-amine dihydrochloride (2.00 g, 5.05 mmol) and anhydrous potassium carbonate (3.07 g, 22.2 mmol) was suspended in DCM:Water (1:1, 30 mL). Chloroacetic acid (0.788 g, 8.34 mmol) and EDAC (1.60 g, 8.34 mmol) were dissolved in DCM (15 mL), stirred for 5 min. and then added to the DCM:water suspension, and stirred vigorous for 2.5 hours. A mixture of chloroacetic acid (0.100 g, 1.0 mmol) and EDAC (0.213 g, 1.1 mmol) was dissolved in DCM and was added to the reaction mixture. The mixture was stirred vigorous for 4 hours. The water phase was removed with a phase separator and another mixture of chloroacetic acid (0.210 g, 2.2 mmol) and EDAC (0.426 g, 2.2 mmol), dissolved in DCM, was added to the organic phase. The mixture was stirred for another 2 hours, concentrated and purified with Biotage Horizon Pioneer® HPFS using a silica cartridge and eluted with EtOAc:MeOH:TEA (100:2:0.2) to give the title compound, (1.53 g, 76%) as a white solid.

¹HNMR (MeOD-d₄) δ 7.72 (d, 2H), 7.65 (d, 2H), 7.27 (m, 2H), 6.35 (m, 1H), 3.98 (s, 2H), 3.65-3.72 (m, 1H), 3.49 (s, 2H), 3.0 (d, 2H), 2.17 (t, 2H), 1.87 (d, 2H), 1.52-1.62 (m, 2H).

MS (ESI) 400.1 (M+1H⁺).

### ii) 2-(3,5-difluorophenoxy)-N-[1-({1-[4-(trifluoromethyl)phenyl]-1H-pyrrol-3-yl}methyl)piperidin-4-yl]acetamide

2-chloro-N-[1-({1-[4-(trifluoromethyl)phenyl]-1H-pyrrol-3-yl}methyl)piperidin-4-yl]acetamide (0.350 g, 0.875 mmol) was dissolved in dry THF (5 mL). 3,5-difluorophenol (0.228 g, 1.751 mmol) and potassium tert-butoxide (0.196 g, 1.751 mmol) was dissolved in dry THF (5 mL) and stirred for 5 min. before adding it to the solution of 2-chloro-N-[1-({1-[4-(trifluoromethyl)phenyl]-1H-pyrrol-3-yl}methyl)piperidin-4-yl]acetamide in THF. The reaction mixture was stirred at rt. over night and was then concentrated and dissolved in DCM (20 mL), washed with water (10 mL), concentrated again and purified with Biotage Horizon Pioneer® HPFS using a silica cartridge with gradient elution with EtOAc:MeoH:TEA (gradient from 100:0:0 to 100:2:0.2) to give the title compound in (218 mg, 51%) as a white solid.

¹HNMR (MeOD-d₄) δ 7.67-7.69 (d, 2H), 7.59-7.61 (d, 2H), 7.22-7.24 (m, 2H), 6.57-6.61 (m, 2H), 6.49-6.55 (m, 1H), 6.32 (s, 1H), 4.47 (s, 2H), 3.72-3.79 (m, 1H), 3.43 (s, 2H), 2.94 (d, 2H), 2.10 (t, 2H), 1.83 (m, 2H), 1.55-1.64 (m, 2H).

¹³CNMR (MeOD-d⁴) δ 168.2, 164.0 (dd, J=16, 246), 160.1 (t, J=16), 143.3, 124.4 (q, J=270), 126.8 (q, J=3.9), 126.7 (q, J=32), 122.1, 119.2, 119.1 118.9, 113.0, 98.6 (dd, J=31.9), 96.7 (t, J=27), 67.4, 54.8, 51.8, 46.8, 30.9.

MS (ESI) 494.1 (M+1H⁺).

### Example 32

### 2-(2,6-diisopropylphenoxy)-N-[1-({1-[4-(trifluoromethyl)phenyl]-1H-pyrrol-3-yl}methyl)piperidin-4-yl]acetamide

2-chloro-N-[1-({1-[4-(trifluoromethyl)phenyl]-1H-pyrrol-3-yl}methyl)piperidin-4-yl]acetamide (0.070 g, 0.175 mmol) was dissolved in dry THF (5 mL). 2,6-diisopropylphenol (0.062 g, 0.350 mmol) and potassium tert-butoxide (0.039 g, 0.350 mmol) was dissolved in dry THF (5 mL) and stirred for 5 min. before adding it to reaction mixture. The reaction mixture was stirred at 50° C. for 30 min then at rt. over night and was then concentrated and purified with a Biotage Horizon Pioneer® HPFS using a silica cartridge with EtOAc:MeOH:TEA (gradient from 100:0:0 to 100:2:0.2) to give the title compound (59 mg, 63%), as a white solid.

¹HNMR (MeOD-d₄) δ 7.70 (d, 2H), 7.63 (d, 2H), 7.26 (d, 2H), 7.10 (s, 3H) 6.35 (m, 1H), 4.23 (s, 2H), 3.83-3.89 (m, 1H), 3.48 (s, 2H); 3.20-3.27 (m, 2H), 2.98 (d, 2H), 2.19 (t, 2H), 1.91 (d, 2H) 1.62-1.72 (m, 2H), 1.20 (d, 12H).

¹³CNMR (MeOD-d⁴) δ 169.2, 152.5, 143.4, 141.5, 126.9 (q, J=3.7), 126.8 (q, J=32), 125.4, 124.5 (q, J=270), 124.2, 122.1, 119.3, 119.1, 118.8, 113.0, 72.9, 54.7, 51.9, 46.5, 31.0, 26.6, 23.3. MS (ESI) 542.7 (M+1H⁺).

Using the method described in Example 32, the compound of Example 33 was similarly prepared from 2-chloro-N-[1-({1-[4-(trifluoromethyl)phenyl]-1H-pyrrol-3-yl}methyl)-piperidin-4-yl]acetamide and 2-isopropylphenol:

### Example 33

### 2-(3-isopropylphenoxy)-N-[1-({1-[4-(trifluoromethyl)phenyl]-1H-pyrrol-3-yl}methyl)piperidin-4-yl]acetamide

¹HNMR (MeOD-d₄) δ 7.70 (d, 2H), 7.62 (d, 2H), 7.24 (m, 2H), 7.17 (t, 1H) 6.84 (m, 2H), 6.73-6.76 (m, 1H), 6.32 (m, 1H), 4.45 (s, 2H), 3.74-3.81 (m, 1H), 3.45 (s, 2H), 2.93 (d, 2H), 2.80-2.96 (m, 1H), 2.13 (t, 2H), 1.83 (d, 2H), 1.55-1.65 (m, 2H), 1.20 (d, 6H).

MS (ESI) 500.6 (M+1H⁺).

### Example 34

### 2-(2-cyanophenoxy)-N-[1-({1-[4-(trifluoromethyl)phenyl]-1H-pyrrol-3-yl}methyl)piperidin-4-yl]acetamide

### 2-chloro-N-[1-({1-[4-(trifluoromethyl)phenyl]-1H-pyrrol-3-yl}methyl)piperidin-4-yl]acetamide (0.050 g, 0.125 mmol), 2-hydroxybenzonitrile (0.022 g, 0.188 mmol), anhydrous potassium carbonate (0.035 g, 0.250 mmol) and potassium iodide (0.010 g, 0.063 mmol) were dissolved in 2-butanone (5 mL) and the mixture was refluxed (70° C.) overnight. The reactions mixture was allowed to cool to rt., and was then concentrated and dissolved in DCM (15 mL) and was washed with 1% Na₂CO₃ aq. solution. The organic phase was dried concentrated and the purified with Biotage Horizon Pioneer® HPFS using a silica cartridge and eluted with EtOAc:MeOH:TEA (100:5:0.1) to give the title compound (37 mg, 62%) as a solid.

¹HNMR (MeOD-d₄) δ 7.70 (d, 2H), 7.58-7.65 (m, 4H), 7.25 (m, 2H) 7.07-7.13 (m, 2H), 6.34 (m, 1H), 4.65 (s, 2H), 3.75-3.83 (m, 1H), 3.47 (s, 2H) 2.92 (d, 2H), 2.20 (t, 2H), 1.90 (m, 2H), 1.59-1.62 (m, 2H).

MS (ESI) 483.4 (M+1H⁺).

Using the method described in Example 34, the compound of Example 35 was similarly prepared from 2-chloro-N-[1-({1-[4-(trifluoromethyl)phenyl]-1H-pyrrol-3-yl}methyl)-piperidin-4-yl]acetamide and isoquinolin-5-ol:

### Example 35

### 2-(isoquinolin-5-yloxy)-N-[1-({1-[4-(trifluoromethyl)phenyl]-1H-pyrrol-3-yl}methyl)piperidin-4-yl]acetaride

¹HNMR (MeOD-d₄) δ 9.2 (bs, 1H), 8.45 (bs, 1H), 8.23 (d, 1H), 7.66-7.76 (m, 5H), 7.59 (t, 1H), 7.33 (m, 2H), 7.16 (d, 1H), 6.40 (m, 1H), 4.76 (s, 2H), 3.91 (m, 1H), 3.73 (s, 2H), 3.16 (d, 2H), 2.51 (t, 2H), 1.99 (m, 2H), 1.67-1.78 (m, 2H).

MS (ESI) 509.2 (M+1H⁺)

### Example 36

### 2-(3,4-difluorophenoxy)-N-[1-({1-[4-(trifluoromethyl)phenyl]-1H-pyrrol-3-yl}methyl)piperidin-4-yl]acetamide

3,4-Difluorophenol (0.059 g, 0.45 mmol) and potassium tert-butoxide (0.051 g, 0.45 mmol) were dissolved in dry THF (2 mL). After stirring for 5 minutes, this solution was added to a solution of 2-chloro-N-[1-({1-[4-(trifluoromethyl)phenyl]-1H-pyrrol-3-yl}methyl)piperidin-4-yl]acetamide (0.090 g, 0.23 mmol, from Example 31) in dry THF (2 mL). The reaction mixture was stirred at 50° C. for 6 hours and was then concentrated. Purification with a Biotage Horizon Pioneer® HPFS using a silica cartridge eluted with EtOAc:MeOH:TEA (100:5:0.1) gave the title compound (0.077 g, 70%) as a solid.

¹H NMR (CDCl₃) δ 7.64 (m, 2H), 7.44 (m, 2H), 7.01-7.11 (m, 3H), 6.74 (m, 1H), 6.60 (m, 1H), 6.35 (d, J=8.0 Hz, 1H), 6.30 (s, 1H), 4.39 (s, 2H), 3.88 (m, 1H), 3.43 (s, 2H), 2.88 (m, 2H), 2.14 (m, 2H), 1.93 (M, 2H), 1.52 (m, 2H).

¹³C NMR (CDCl₃) δ 166.9, 153.6 (m), 150.8 (dd, J=15.2 Hz, J=250 Hz), 146 (dd, J=12.4 Hz, J=242.5 Hz), 143.2, 127.4 (q, J=32.9 Hz), 127.1 (q, J=3.9 Hz), 124.2 (q, J=273 Hz), 123.7, 119.7, 119.2, 118.4, 117.8 (d, J=20.2 Hz), 113, 110.1 (m), 105.1 (d, J=20.6 Hz), 68.3, 55.4, 52.2, 46.6, 32.3.

MS (ESI) 494.3 (M+1H⁺), MS (ESI) 492.0 (M−1H⁺).

### Example 37

### 2-[(5-chloropyridin-2-yl)oxy]-N-[1-({1-[4-(trifluoromethyl)phenyl]-1H-pyrrol-3-yl}methyl)piperidin-4-yl]acetamide

2-chloro-5-hydroxypyridine (0.162 g, 1.25 mmol) and potassium tert-butoxide (0.140 g, 1.25 mmol) were dissolved in dry THF (10 mL). After stirring for 5 minutes, this solution was added to a solution of 2-chloro-N-[1-({1-[4-(trifluoromethyl)phenyl]-1H-pyrrol-3-yl}methyl)piperidin-4-yl]acetamide (0.250 g, 0.63 mmol, from Example 31) dissolved in dry THF (10 mL). The reaction mixture was stirred at rt. over night. The solution was concentrated and dissolved in CH₂Cl₂ (15 mL), washed with saturated Na₂CO₃ aqueous solution (10 mL), concentrated and purified with a Biotage Horizon Pioneer® HPFS using a silica cartridge eluted with EtOAc:MeOH:TEA (gradient from 100:2:0.2 to 100:5:0.5), followed by purification by HPLC, to give the title compound (0.136 g, 44%) as a white solid.

¹H NMR (CDCl₃) δ 8.12 (d, J=7.5 Hz, 1H), 7.84 (d, J=3.0 Hz, 1H), 7.76 (s, 4H), 7.46 (dd, J=9.8 Hz, 1H), 7.43 (t, J=2.4 Hz, 1H), 7.37 (bs, 1H), 6.37 (d, J=9.6 Hz, 1H), 6.25 (bs, 1H), 4.46 (s, 2H), 3.44-3.55 (m, 1H), 3.28 (bs, 2H), 2.81 (bs, 2H), 1.97 (m, 2H), 1.70 (bs, 2H), 1.39 (dt, J=10.8 Hz, 2H).

MS (ESI) 493.2 (M+1H⁺), MS (ESI) 491.4 (M−1H⁺).

### Example 38

### 2-(3-chlorophenoxy)-N-[1-({1-[6-(trifluoromethyl)pyridin-3-yl]-1H-pyrrol-3-yl}methyl)piperidin-4-yl]acetamide

2-(3-chlorophenoxy)-N-piperidin-4-ylacetamide (0.120 g, 0.45 mmol, from Example A) and 1-[6-(trifluoromethyl)pyridin-3-yl]-1H-pyrrole-3-carbaldehyde (0.129 g, 0.54 mmol, from Example P) were dissolved in DCM (7.5 mL) in a 16 mL vial and stirred for 10 minutes. MP-BH(OAc)₃ (0.531 g, 1.12 mmol) was then added and the vial was loosely sealed with a cap and the reaction left stirring for 2 hours. The reaction mixture was filtered and the filtrate was washed with MeOH (2 mL), concentrated and purified with a Biotage Horizon Pioneer® HPFS using a silica cartridge eluted with EtOAc:MeOH:TEA (gradient from 100:2:0.2) to give the title compound (0.167 g, 76%) as a white solid after evaporation from MeCN.

¹H NMR (CDCl₃) δ 8.78 (s, 1H), 7.78 (d, J=9.1 Hz, 1H), 7.71 (d, J=9.1 Hz, 1H), 7.21 (m, 1H), 7.08 (s, 1H), 7.04 (s, 1H), 6-98 (bs, 1H), 6.91 (bs, 1H), 6.78 (d, J=9.1 Hz, 1H), 6.36 (bs, 2H), 4.43 (bs, 2H), 3.88 (bs, 1H), 3.43 (s, 2H), 2.86 (m, 2H), 2.14 (m, 2H), 1.93 (m, 2H), 1.51 (m, 2H).

¹³C NMR (CDCl₃) δ 166.9, 157.9, 141.2, 138.8, 135.4, 130.8, 127.2, 124.9, 122.6, 121.6, 119.1, 118.1, 115.7, 114.2, 113.0, 67.8, 55.3, 52.2, 46.6, 32.2.

MS (ESI⁺) 493.1 (M+H⁺), MS (ESI−) 491.1 (M−H⁺).

### Example 39

### 2-(biphenyl-3-yloxy)-N-[1-({1-[4-(trifluoromethyl)phenyl]-1H-pyrrol-3-yl}methyl)piperidin-4-yl]acetamide, acetate salt

3-phenylphenol (0.021 g, 0.125 mmol), and potassium tert-butoxide (0.014 g, 0.125 mmol) were dissolved in dry THF (2 mL). After stirring for 5 minutes, this solution was added to a solution of 2-chloro-N-[1-({1-[4-(trifluoromethyl)phenyl]-1H-pyrrol-3-yl}methyl)piperidin-4-yl]acetamide (0.025 g, 0.063 mmol, from Example 31) dissolved in dry THF (2 mL). The reaction mixture was stirred at rt. over night. The reactions mixture was concentrated and dissolved in CH₂Cl₂ (2 mL) and washed with Na₂CO₃ (5 g/100 mL) aq. solution. Purification with HPLC gave the title compound (0.009 g, 23%).

MS (ESI) 534.4 (M+1H⁺), MS (ESI) 532.4 (M−1H⁺).

### Example 40

### 2-(4-chlorophenoxy)-2-methyl-N-[1-({1-[4-(trifluoromethyl)phenyl]-1H-pyrrol-3-yl}methyl)piperidin-4-yl]propanamide, acetate salt

1-({1-[4-(trifluoromethyl)phenyl]-1H-pyrrol-3-yl}methyl)piperidin-4-amine dihydrochloride (0.025 g, 0.063 mmol, from Example 29) and 2-(4-chlorophenoxy)-2-methylpropanoic acid (0.016 g, 0.076 mmol) were dissolved in DMF (2 mL) and N,N-Diisopropylethylamine (0.029 g, 0.23 mmol) was added to the stirred solution. HATU (0.029 g, 0.076 mmol) was added and the reaction mixture was stirred at rt over night. The reactions mixture was concentrated and purified with HPLC to give the title compound (0.020 g, 56%).

MS (ESI) 520.3 (M+1H⁺), MS (ESI) 518.6 (M−1H⁺).

### Example 41

### 2-(3-chlorophenoxy)-N-[1-({1-[4-(trifluoromethyl)phenyl]-1H-pyrrol-3-yl}methyl)azetidin-3-yl]acetamide

### a) tert-butyl[1-({1-[4-(trifluoromethyl)phenyl]-1H-pyrrol-3-yl}methyl)azetidin-3-yl]carbamate

tert-butyl azetidin-3-ylcarbamate (200 mg, 1.16 mmol) and 1-[4-(trifluoromethyl)phenyl]-1H-pyrrole-3-carbaldehyde (1.2 eq) were dissolved/suspended in DCM (10 mL) in a 16 mL vial and stirred for 10 minutes. MP-BH(OAc)₃ (2.5 eq) was then added and the vial loosely sealed with a cap and the reaction left for 3 h. The reaction was filtered washing with DCM (2 mL) and the filtrate evaporated in vacuo to yield a brown oil. Flash chromatography on the Biotage 40 g column using isocratic EtOAc:MeOH:TEA (100:3:0.2) gave the product as a white solid (334 mg, 73%).

MS (ESI+): 396.1 (M+H⁺); MS (ESI−): 394.06 (M−H⁺)

### b) 2-chloro-N-[1-({1-[4-(trifluoromethyl)phenyl]-1H-pyrrol-3-yl}methyl)azetidin-3-yl]acetamide

To tert-butyl[1-({1-[4-(trifluoromethyl)phenyl]-1H-pyrrol-3-yl}methyl)azetidin-3-yl]carbamate (334 mg, 0.85 mmol) was added 4M HCl in dioxane (10 mL) and stirred for 1 hour. To the resultant solution/precipitate was added ether (50 mL) to cause further precipitation. Filtration and washing with ether (100 mL) yielded a white solid. After drying in vacuo the solid was suspended in DCM (5 mL) and shaken with 10% Na₂CO₃ (5 mL). The organic layer was separated over a phase separator washing through with DCM (5 mL). To the pooled DCM fractions was added a preformed solution of chloroacetic acid (1.2 eq) and EDC.HCl (1.2 eq) in DCM (5 mL). The reaction was stirred for 2 hours, concentrated in vacuo and the oily residue purified by flash chromatography on a 40 g Biotage column using EtOAc/MeOH/TEA (100/2/0.2) to yield a white solid (193 mg, 62%).

HPLC purity 98%

### c) 2-(3-chlorophenoxy)-N-[1-({1-[4-(trifluoromethyl)phenyl]-1H-pyrrol-3-yl}methyl)azetidin-3-yl]acetamide

To 3-chlorophenol (2 eq) dissolved in dry THF (1 mL) was added tert-butoxide (2 eq) and agitated for 5 minutes. In turn this was added to a solution of 2-chloro-N-[1-({1-[4-(trifluoromethyl)phenyl]-1H-pyrrol-3-yl}methyl)azetidin-3-yl]acetamide (140 mg, 0.38 mmol), in THF (4 mL) heated to 80 C and stirred for 1 hour. The reaction mixture was evaporated in vacuo and dissolved in MeOH/DCM.

Flash chromatography on the Biotage 9 g column using isocratic EtOAc:MeOH:TEA (100:1:0.1) gave the product as a white solid after evaporation from ether (159 mg, 91%).

¹HNMR (CDCl₃) δ 3.1 (t, 2H), 3.5 (s, 2H), 3.65 (t, 2H), 4.45 (s, 2H), 4.6 (tt, 1H), 6.3 (s, 1H), 6.8 (dd, 1H), 6.95-7.05 (m, 5H), 7.2 (t, 1H), 7.45 (d 2H), 7.65 (d, 2H).

¹³CNMR (CDCl₃): δ 167.4, 158.0, 143.2, 135.4, 130.8, 127.4 (q, J=33), 127.1 (q, J=3.3), 124.3 (q, J=271), 122.9, 122.6, 119.8, 119.5, 117.6, 115.8, 113.0, 112.1, 67.6, 61.4, 55.9, 40.8

MS (ESI⁺): 464.05 (M+H⁺); MS (ESI−): 462.00 (M−H⁺)

### Example 42

### 2-(diphenylmethoxy)-N-[1-({1-[4-(trifluoromethyl)phenyl]-1H-pyrrol-3-yl}methyl)piperidin-4-yl]acetamide

2-chloro-N-[1-({1-[4-(trifluoromethyl)phenyl]-1H-pyrrol-3-yl}methyl)piperidin-4-yl]acetamide (0.075 g, 0.19 mmol) was dissolved in dry THF (2 mL). Diphenylmethanol (0.069 g, 0.38 mmol) and potassium tert-butoxide (0.042 g, 0.38 mmol) was dissolved in dry THF (2 mL) and stirred for 5 min. before adding it to the solution of 2-chloro-N-[1-({1-[4-(trifluoromethyl)phenyl]-1H-pyrrol-3-yl}methyl)piperidin-4-yl]acetamide in THF. Stirred at 50° C. for 12 hours. Concentrated and purified with Biotage Horizon Pioneer® HPFS using a silica cartridge with gradient elution with n-Heptane/EtOAc:MeOH:TEA (100:5:0.1) to give the title compound in 0.059 g (58%).

¹H NMR (CD₃OD) δ 7.70 (d, 2H, J=8.7 Hz), 7.61 (d, 2H, J=87 Hz), 7.33-7.38 (m, 4H), 7.27-7.33 (m, 4H), 7.20-7.26 (m, 4H), 6.33 (s, 1H), 5.47 (s, 1H), 3.93 (s, 2H), 3.68-3.77 (m, 1H), 3.44 (s, 2H), 2.88 (m, 2H), 2.14 (t, 2H, J=11.1 Hz), 1.82 (m, 2H), 1.54 (m, 2H).

¹³C NMR (CD₃OD) δ 170.4, 143.4, 141.5, 128.4, 127.7, 127.4 (q, J=28.5 Hz), 126.9, 126.8 (q, J=3.3 Hz), 124.4 (q, J=270 Hz), 122.1, 119.2, 119.1, 118.9, 113.0, 84.4, 68.2, 54.7, 51.6, 46.3, 30.9.

MS (ESI+) 548.5 (M+1H⁺), MS (ESI−) 546.2 (M−1H⁺).

### Example 43

### 2-(3-chlorophenoxy)-N-[(3S,4S)-3-fluoro-1-({1-[4-(trifluoromethyl)phenyl]-1H-pyrrol-3-yl}methyl)piperidin-4-yl]acetamide and 2-(3-chlorophenoxy)-N-[(3R,4R)-3-fluoro-1-({1-[4-(trifluoromethyl)phenyl]-1H-pyrrol-3-yl}methyl)piperidin-4-yl]acetamide

### a) tert-butyl 4-[(trimethylsilyl)oxy]-3,6-dihydropyridine-1(2H)-carboxylate

To a solution of tert-butyl 4-oxopiperidine-1-carboxylate (5 g, 20-1 mmol) in dry DMF (20 mL) was added TMSCl (1.2 eq), TEA (2.4 eq, fresh) and the mixture stirred at 80 C for 18 h under N₂. The mixture was diluted with hexane (100 mL) and washed with 10% NaHCO₃ (aq) (2×100 mL). The organic layer was dried over MgSO₄ and concentrated in vacuo. Column chromatography using EtOAc/Heptane (1:9) gave the product as a colourless oil.

¹HNMR (CDCl₃) δ 0.2 (s, 9H), 1.45 (s, 9H), 2.1 (br.s, 2H), 3.5 (m, 2H), 3.85 (s, 2H), 4.7 (s, 1H).

### b) tert-butyl (3S,4S)-4-{[(3-chlorophenoxy)acetyl]amino}-3-fluoropiperidine-1-carboxylate and tert-butyl (3R,4R)-4-{[(3-chlorophenoxy)acetyl]amino}-3-fluoropiperidine-1-carboxylate

To a stirred solution of tert-butyl 4-[(trimethylsilyl)oxy]-3,6-dihydropyridine-1(2H)-carboxylate (3.4 g, 12.5 mmol) in Dry MeCN (15 mL) was added, under N₂, Selectfluor reagent (1.1 eq) and the mixture stirred for 2 hours at rt. The reaction mixture was then poured into EtOAc (50 mL) and washed with 1% NaHCO₃ (aq) 50 mL and saturated brine (50 mL). The organics were dried over MgSO₄, filtered and concentrated in vacuo. Flash Chromatography on a Biotage Column (40 g) using EtOAc/Heptane gradient 20-100% gave the fluorinated intermediate as a yellowish oil (1.6 g, 7.4 mmol). The oil was taken up in methanol (20 mL) to which was added ammonium acetate (7 eq) and stirred for 2 h at room temperature. Sodium cyanoborohydride (1.2 eq) was then added and the reaction stirred for a further 4 hours. The reaction mixture was concentrated to dryness and the organics extracted with ethylacetate (2×50 mL) from a 1% aq solution of Na₂CO₃ (100 mL). The EtOAc was washed with brine, dried over Na₂SO₄ and evaporated in vacuo. The resulting amino derivative was dissolved in DCM (10 mL) to which was added a preformed solution of (3-chlorophenoxy)acetic acid (1 eq), EDC.HCl (1 eq) and the mixture stirred for 2 hours at rt. The mixture was shaken with 0.1 M KHSO₄ (aq) (50 mL), filtered over a phase separator and concentrated in vacuo. The resulting oil was flash chromatographed using the Horizon Biotage 40 g column with a gradient of EtOAC/Heptane 10-50%. The diastereoisomers were separated, the quicker eluting being the trans relative isomers, and isolated as a white solid. (310 mg, 11%). ¹HNMR (CDCl₃) δ 1.45 (s, 9H), 4.5 (s, 2H), 2.2-2.6 (m, 1H), 2.1 (m, 1H), 2.9 (m, 2H), 3.8-4.6 (m, 4H), 6.6 (d, 1H), 6.8 (d, 1H), 6.9 (s, 1H), 7.0 (d, 1H), 7.2 (t, 1H).

### c) 2-(3-chlorophenoxy)-N-[(3S,4S)-3-fluoro-1-({1-[4-(trifluoromethyl)phenyl]-1H-pyrrol-3-yl}methyl)piperidin-4-yl]acetamide and 2-(3-chlorophenoxy)-N-[(3R,4R)-3-fluoro-1-({1-[4-(trifluoromethyl)phenyl]-1H-pyrrol-3-yl}methyl)piperidin-4-yl]acetamide

tert-butyl (3S,4S)-4-{[(3-chlorophenoxy)acetyl]amino}-3-fluoropiperidine-1-carboxylate and tert-butyl (3R,4R)-4-{[(3-chlorophenoxy)acetyl]amino}-3-fluoropiperidine-1-carboxylate (140 mg, 0.362 mmol) was dissolved in 4M HCl in Dioxane (10 mL), stirred at rt for 2 hours and the solvents removed in vacuo.

The resulting oil, DIPEA (2 eq) and 1-[4-(trifluoromethyl)phenyl]-1H-pyrrole-3-carbaldehyde (1.2 eq) were dissolved in DCM (7.5 mL) in a 16 mL vial and stirred for 10 minutes. MP-BH(OAc)₃ (3 eq) was added and the vial loosely sealed with a cap and the reaction stirred for 3 h at rt. The reaction was filtered washing with DCM/MeOH (4 mL) and the filtrate evaporated in vacuo to yield a yellow oil.

Flash chromatography on the Biotage 9 g column using gradient EtOAc:MeOH:TEA (100:5:0.5) 10-100% over 540 mL against EtOAC gave the product as a colourless oil (146 mg, 68%).

¹HNMR (CDCl₃) δ 1.6 (m, 1H), 2.0-2.2 (m 3H), 2.8 (br.d., 1H), 3.3 (dt, 1H), 3.4-3.6 (m, 2H), 4.0-4.1 (m, 1H), 4.3-4.6 (m, 1H), 4.45 (s, 2H), 6.3 (s, 1H), 6.5 (d, 1H), 6.8 (dd, 1H), 6.95 (s, 1H), 7.0 (m, 2H), 7.1 (s, 1H), 7.2 (t, 1H), 7.45 (d 2H), 7.65 (d, 2H).

¹³CNMR (CDCl₃): δ 167.9, 158.1, 143.2, 135.5, 130.8, 127.4 (q, J=33.3), 127.1 (q, J=3.3), 124.3 (q, J=270), 123.1, 122.7, 119.8, 119.4, 118.4, 115.9, 113.1, 112.9, 90.4 (d, J=185), 67.8, 55.4, 56.2 (d, J=26), 54.7, 51.6 (d, J=16), 51.4, 30.1 (d, J=7).

MS (ESI+): 510.13 (M+H⁺); MS (ESI−): 508.09 (M−H⁺)

### Example 44

### 2-(3,4-difluorophenoxy)-N-[1-({1-[4-(trifluoromethyl)phenyl]-1H-pyrrol-3-yl}methyl)pyrrolidin-3-yl]acetamide

2-(3,4-difluorophenoxy)-N-pyrrolidin-3-yl acetamide (0.075 g, 0.29 mmol), 1-[4-(trifluoromethyl)phenyl]-1H-pyrrole-3-carbaldehyde (0.084 g, 0.35 mmol) and MP-triacetoxyborohydride (0.35 g, 0.73 mmol) was dissolved in methylene chloride (4 mL) and stirred for 4 hours. Added water (2 mL) and separated on phase separator. Concentrated and purified with Biotage Horizon Pioneer® HPFS using a silica cartridge elution with EtOAc:MeOH:TEA (100:2:0.2) to give the title compound in 0.120 g (85%).

¹H NMR (CDCl₃) δ 7.63 (d, 2H, J=8.9 Hz), 7.42 (d, 2H, J=8.9 Hz), 7.00-7.10 (m, 3H), 6.81 (d, 1H, J=8.1 Hz), 6.74 (m, 1H), 6.59 (m, 1H), 6.29 (s, 1H), 4.54 (m, 1H), 4.37 (s, 2H), 3.54 (q, 2H, J=12.8 Hz), 2.94 (m, 1H), 2.57-2.70 (m, 2H), 2.32 (m, 2H) 1.60-1.70 (m, 1H).

¹³C NMR (CDCl₃) δ 166.9, 153.6 (m), 150.7 (dd, J=13.8 Hz, J=249.1 Hz), 146 (dd, J=14.8 Hz, J=243.8 Hz), 143.2, 127.4 (q, J=30.7 Hz), 127.0 (q, J=3.8 Hz) 124.3, 124.3 (q, J=270.9 Hz), 119.7, 119.3, 117.9, 117.7 (d, J=19.2 Hz), 112.7, 110.1 (m), 105.0 (d, J=20.3 Hz), 68.2, 60.7, 52.9, 52.2, 48.5, 32.7.

MS (ESI⁺) 480.7 (M+1H⁺), MS (ESI−) 478.3 (M−1H⁺).

The enantiomers of the compound of Example 44 were separated by multiple injections (24 mg in 2 ml EtOH) on a Chiralpak AS column (250×20 mm I.D.) with EtOH/TEA (100/0.1) as the mobile phase at 40° C. E.e. analysis was performed on a Chiralpak AS column (4.6×250 mm I.D.) at ambient temperature and detection at 225 nm.

### Example 44 a

### (+) 2-(3,4-difluorophenoxy)-N-[1-({1-[4-(trifluoromethyl)phenyl]-1H-pyrrol-3-yl}methyl)pyrrolidin-3-yl]acetamide

Eluent 1, 35 mg (72%) 99.8% e.e., [(XID²⁰=+8.1 (c 1.0, CH₃CN)

### Example 44 b

### (−) 2-(3,4-difluorophenoxy)-N-[1-({1-[4-(trifluoromethyl)phenyl]-1H-pyrrol-3-yl}methyl)pyrrolidin-3-yl]acetamide

Eluent 2, 37 mg (77%) 99.1% e.e., [α]_{D}²⁰=−8.3 (c 1.0, CH₃CN)

### Example 45

### 2-(3-chlorophenoxy)-N-{1-[(1-{4-[(trifluoromethyl)sulfonyl]phenyl}-1H-pyrrol-3-yl)methyl]piperidin-4-yl}acetamide

2-(3-chlorophenoxy)-N-piperidin-4-ylacetamide (60 mg, 0.22 mmol) and 1-{4-[(trifluoromethyl)sulfonyl]phenyl}-1H-pyrrole-3-carbaldehyde (1.2 eq) were dissolved in DCM (7.5 mL) and left to stir for 10 minutes. MP-BH(OAc)₃ (2.5 meq) was added and the reaction stirred for a further 2 h at ambient temperature. The reaction was filtered, washed through with DCM/MeOH (1:1, 4 mL) and the filtrate concentrated in vacuo. Flash silica chromatography on a 9 g Biotage cartridge eluting with a gradient of EtOAc/MeOH/TEA (100/5/0.5) 10-100% over 540 mL against EtOAc yielded the product as an oil (85 mg, 65%, 95% purity).

¹HNMR (CDCl₃) δ 8.05 (d, 2H), 7.6 (d, 2H), 7.2 (t, 1H), 7.15 (s, 1H), 7.1 (s, 1H), 7.0 (d, 1H), 6.9 (s, 1H), 6.8 (s, 1H), 6.4 (m, 2H), 4.45 (s, 2H), 3.9 (m, 1H), 3.40 (s, 2H), 2.85 (d, 2H), 2.11 (t, 2H), 1.9 (m, 2H), 1.50 (m, 2H).

¹³CNMR (CDCl₃): δ 167.0, 158.0, 146.7, 135.5, 133.1, 130.8, 126.4, 125.5, 122.6, 119.6, 119.2, 118.4, 118.1, 115.8, 114.6, 113.0, 67.7, 55.3, 52.2, 46.5, 32.3

MS (ESI+): 556.5 (M+H⁺); MS (ESI−): 554.1 (M−H⁺)

### Example 46

### 2-(3-chlorophenoxy)-N-(1-{[1-(2,2-difluoro-1,3-benzodioxol-5-yl)-1H-pyrrol-3-yl]methyl}piperidin-4-yl)acetamide

2-(3-chlorophenoxy)-N-piperidin-4-ylacetamide (60 mg, 0.22 mmol) and 1-(2,2-difluoro-1,3-benzodioxol-5-yl)-1H-pyrrole-3-carbaldehyde (1.2 eq) were dissolved in DCM (7.5 mL) and left to stir for 10 minutes. MP-BH(OAc)₃ (2.5 meq) was added and the reaction stirred for a further 2 h at ambient temperature. The reaction was filtered, washed through with DCM/MeOH (1:1, 4 mL) and the filtrate concentrated in vacuo. Flash silica chromatography on a 9 g Biotage cartridge eluting with a gradient of EtOAc/MeOH/TEA (1001510.5) 10-100% over 540 mL against EtOAc yielded the product as a white solid (82 mg, 71%, 98% purity).

¹HNMR (CDCl₃) δ 7.2 (t, 1H), 7.1 (m, 3H), 7.0 (d, 1H), 6.9 (m, 3H), 6.8 (d, 1H), 6.4 (d, 1H), 6.3 (s, 1H), 4.45 (s, 2H), 3.9 (m, 1H), 3.40 (s, 2H), 2.85 (d, 2H), 2.11 (t, 2H), 1.9 (m, 2H), 1.50 (m, 2H).

¹³CNMR (CDCl₃): δ 167.0, 158.0, 144.5, 141.6, 137.4, 135.4, 132.0 (t, J=260), 130.8, 123.0, 122.6, 119.8, 119.1, 115.7, 115.6, 113.0, 112.5, 110.0, 103.3, 67.7, 55.4, 52.1, 46.5, 32.3

MS (ESI+): 556.5 (M+H⁺); MS (ESI−): 554.1 (M−H⁺)

### Pharmacological Properties

MCH1 Receptor Radioligand Binding.

Assays were performed on membranes prepared from CHO—K1 cells expressing the human Melanin concentrating hormone receptor 1 (MCH1r). Assays were performed in a 96-well plate format in a final reaction volume of 200 μl per well. Each well contained 6 μg of membrane proteins diluted in binding buffer (50 mM Tris, 3 mM MgCl₂, 0.05% bovine serum albumin and the radioligand ¹²⁵I-MCH (IM344 Amersham) was added to give 10 000 cpm (counts per minute) per well. Each well contained 2 μl of the appropriate concentration of competitive antagonist prepared in DMSO and left to stand at 30° C. for 60 minutes. Non-specific binding was determined as that remaining following incubation with 1 μM MCH (Melanin concentrating hormone, H-1482 Bachem). The reaction was terminated by transfer of the reaction to GF/A filters using a Micro96 Harvester (Skatron Instruments, Norway). Filters were washed with assay buffer. Radioligand retained on the filters was quantified using al450 Microbeta TRILUX (Wallac, Finland).

Non-specific binding was subtracted from all values determined. Maximum binding was that determined in the absence of any competitor following subtraction of the value determined for non-specific binding. Binding of compounds at various concentrations was plotted according to the equation

y=A+((B−A)/1+((C/x)̂D)))

and IC₅₀ estimated where
A is the bottom plateau of the curve i.e. the final minimum y value
B is the top of the plateau of the curve i.e. the final maximum y value
C is the x value at the middle of the curve. This represents the log EC50 value when A+B=100
D is the slope factor. x is the original known x values. y is the original known y values.

The compounds exemplified herein had an IC₅₀ of less than 1 μM in the abovementioned human MCHr binding assay. Preferred compounds had an activity of less then 0.3 μM. For instance, the following IC₅₀ values were obtained for the compounds of the following examples:
Example 3, 0.167 μM
Example 8, 0.105 μM
Example 29, 0.066 μM
Example 41, 0.039 μM
Example 44, 0.027 μM

Assays may also be performed on membranes prepared from HEK293 cells stably expressing the rat Melanin concentrating hormone receptor 1 (MCH1r) (Lembo et al. Nature Cell Biol. 1 267-271). Assays were performed in a 96-well plate format in a final reaction volume of 2001 per well. Each well contained 5 μg of membrane proteins diluted in binding buffer (50 mM Tris, 3 mM MgCl₂, 0.05% bovine serum albumin and the radioligand ¹²⁵I-MCH (IM344 Amersham) was added to give 10 000 cpm (counts per minute) per well. Each well contained 2 μl of the appropriate concentration of competitive antagonist prepared in DMSO and left to stand at room temperature for 60 minutes. Non-specific binding was determined as that remaining following incubation with 1 μM MCH (Melanin concentrating hormone, H-1482 Bachem). The reaction was terminated by transfer of the reaction to GF/A filters using a Micro96 Harvester (Skatron Instruments, Norway). Filters were washed with assay buffer. Radioligand retained on the filters was quantified using al450 Microbeta TRILUX (Wallac, Finland).

### MCH1 Functional Assay

Membranes expressing recombinant hMCHr (5.45 pmol/mg protein; Euroscreen) were prepared in assay buffer (50 mM HEPES, 100 mM NaCl, 5 mM MgCl₂, 1 mM EDTA, 200 μM DTT, 20 μM GDP (Sigma) containing 0.1 μg/ml BSA, pH7.4) before assay. The assays were performed using membranes at 6 μg/well in an assay volume of 200 μl and the appropriate concentrations of compounds prepared in DMSO. The reaction was started by addition of 0.056 nM [³⁵S]GTPγS (Specific activity >1000 Ci/mmol; Amersham) and an ED₈₀ concentration of MCH (determined for each membrane and each MCH batch). Non-specific binding was determined using 20 μM non-radiolabelled GTPγS. Plates were incubated for 45 min at 30° C. Free and bound GTPγS were separated by filtration binding using GF/B filter mats presoaked in wash buffer (50 mM Tris, 5 mM MgCl₂, 50 mM NaCl, pH 7.4) using a Micro96 cell harvester (Skatron Instruments) and the filters then dried at 50° C. before counting using al450 Microbeta TRILUX (Wallac).

Data are means ±SD for experiments performed in triplicate. IC₅₀ values of antagonists were determined using non-linear regression analysis of concentration response curves using Activity Base. For instance, the following IC₅₀ values were obtained for the compounds of the following examples:
Example 3, 0.045 μM
Example 8, 0.111 μM
Example 29, 0.066 μM

### Pharmacodynamic Effect in Rat

Male Wistar-Hanover rats (Charles River, 300-350 grams) were acclimated to individually housing in conventional cages (Makrolon III) with 12:12 hour light-dark photoperiod (lights on at 06.00) in a temperature (20-22° C.) and humidity (40-60%) controlled room. R-3 lab chow (Lactanin, Vadstena, Sweden) and tap water from bottles were allowed ad libidum. At 16.00 on the day before experiments, animals were weighed & food (but not water) was removed. At 08.00 on experiment day, animals were weighed & compound (i.p. amorphous nanoparticle formulation, 5 ml/kg) or vehicle (3-10% DMA depending on compound formulation) administered. Animals were returned to their home cages & given access to a weighed amount of food. This food was then re-weighed 1, 2, 4, 6 & 24 hours later, and food consumption calculated by the difference from initial food weight. For example, the compound of Example 34 (16.7 μmol/kg) reduced food intake by 20% during the time interval 0-4 h.

Animals were further weighed at the 24-hr timepoint, and change in body weight over the treatment period was calculated. Compounds of the invention significantly decreased weight gain over the 24-hr observation period.

### Pharmacodynamic Effect in Mouse

Female C57B16 mice (19-21 g) were singly housed for 7-days with ad libitum access to a “bland-paste” made from normal laboratory chow (R-3 Lactanin, Vadstena, Sweden) or to a “palatable-paste” of similar consistency containing oatmeal, butter, sugar, cocoa powder, cocoa butter & peanut butter. The day before the experimental day, food was removed for 12 hours. At 09.00 on experiment day, animals were weighed & compound (i.p. amorphous nanoparticle formulation, 10 ml/kg) or vehicle (0.1% Tween 80 or <5% DMA, depending on compound formulation) administered. Animals were returned to their home cages & given access to weighed amounts of both bland & palatable pastes. This food was then re-weighed 2, 4 and 6 hours later, and consumption of each food type calculated by the difference from initial food weight. Animals were further weighed at 24-hrs after administration, and change in body weight over the treatment period was calculated. Compounds of the invention gave a significant decrease in food intake, the effect being more pronounced on the reduction of intake of “palatable-paste” food. Compounds of the invention also significantly decreased weight gain over the 24-hr observation period.

1. A compound of formula I 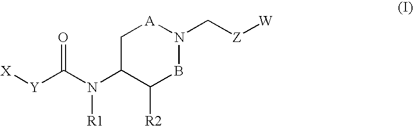
wherein X represents phenyl, naphthyl, pyrrolyl, imidazolyl, furyl, thienyl, thiazolyl, isothiazolyl, thiadiazolyl, pyrazolyl, oxazolyl, isoxazolyl, pyridyl, pyrazinyl, pyrimidinyl, pyridazinyl, quinolinyl, isoquinolyl, quinazolyl, indolyl, benzofuranyl, benzo[b]thienyl or benzimidazolyl,
wherein each X is optionally substituted by one or more of the following: cyano, halo, a C₁₋₄ alkyl group optionally substituted by one or more fluoro, a C₁₋₄ alkoxy group optionally substituted by one or more fluoro, a group CONR^{a}R^{b} in which R^{a} and R^{b} independently represent a C₁₋₃ alkyl group, phenyl, phenoxy, 2-pyridyl or 3-pyridyl, wherein the aromatic substituents (i.e. phenyl, phenoxy, 2-pyridyl or 3-pyridyl) may optionally be substituted by fluoro, chloro or cyano, or
X represents a diphenylmethyl or a dipyridinylmethyl group, optionally independently substituted at the aryl group(s) by one or more cyano, halo, trifluoromethoxy, difluoromethoxy or trifluoromethyl,
Y is OCH₂, SCH₂ (wherein the heteroatom is connected to X), CH₂CH₂ or CH═CH, wherein each carbon in Y is optionally substituted by 1 or 2 methyl groups and/or 1 or 2 fluoro,
R¹ represents H or a C₁₋₄alkyl group,
A represents (CH₂)ₙ, wherein n is 0 or 1 and B represents (CH₂)ₘ, wherein m is 0 or 1,
R² represents H or, when A and B are identical and represents CH₂, R₂ represents H or F,
Z represents phenyl or a heterocyclic group selected from thienyl, furyl, pyridyl, pyrazinyl, pyridazinyl, pyrrolyl, imidazolyl, thiazolyl, isothiazolyl, thiadiazolyl, pyrimidinyl, pyrazolyl, oxazolyl, isoxazolyl wherein each Z is optionally substituted by one or more of the following: cyano, halo, a C₁₋₄ alkyl group optionally substituted by one or more fluoro, a C₁₋₄ alkoxy group optionally substituted by one or more fluoro,
W represents phenyl or a heterocyclic group selected from thienyl, furyl, pyridyl, pyrazinyl, pyridazinyl, pyrrolyl, imidazolyl, thiazolyl, isothiazolyl, thiadiazolyl, pyrimidinyl, pyrazolyl, oxazolyl, isoxazolyl wherein each W is optionally substituted by one or more of the following: cyano, halo, a C₁₋₄ alkyl group optionally substituted by one or more fluoro, a C₁₋₄ alkoxy group optionally substituted by one or more fluoro, or W is optionally substituted with a trifluoromethylsulfonyl or a 2,2-difluoro-1,3-dioxolane ring (fused with two adjacent aromatic carbon atoms in W),
as well as tautomers, optical isomers and racemates thereof as well as pharmaceutically acceptable salts thereof,
with the proviso that 2-(4-chlorophenoxy)-N-{1-[4-(1,2,3-thiadiazol-4-yl)benzyl]piperidin-4-yl}acetamide is excluded.

2. A compound according to claim 1, in which X represents a phenyl or pyridyl group substituted with one or more cyano, fluoro, chloro, trifluoromethoxy, difluoromethoxy or trifluoromethyl, or X represents a diphenylmethyl or a dipyridinylmethyl group, optionally substituted at the aryl group(s) by one or more cyano, fluoro, chloro, trifluoromethoxy, difluoromethoxy or trifluoromethyl,
Y is OCH₂ or SCH₂ (both in which the heteroatom is connected to X), CH₂CH₂ or CH═CH,
R¹ is hydrogen or methyl
A represents (CH₂)ₙ, wherein n is 0 or 1 and B represents (CH₂)ₘ, wherein m is 0 or 1,
R² represents H or, when A and B are identical and represents CH₂, R₂ represents H or F,
Z is phenyl or a heterocyclic group selected from thienyl, furyl, pyrrolyl wherein each Z is optionally substituted by cyano, fluoro, chloro or trifluoromethyl,
W represents phenyl or a heterocyclic group selected from thienyl, furyl, pyridyl, pyrazinyl, pyridazinyl, pyrrolyl, imidazolyl, thiazolyl, isothiazolyl, thiadiazolyl, pyrimidinyl, pyrazolyl, oxazolyl, isoxazolyl wherein each W is optionally substituted by one or more of the following: cyano, fluoro, chloro, trifluoromethoxy, difluoromethoxy or trifluoromethyl, or with one trifluoromethylsulfonyl or one 2,2-difluoro-1,3-dioxolane ring (fused with two adjacent aromatic carbon atoms in W),
as well as pharmaceutically acceptable salts, thereof.

3. A compound according to claim 1, wherein X represents naphthyl or a heteroaryl ring selected from quinolinyl, isoquinolyl, quinazolyl, indolyl, benzofuranyl, benzo[b]thienyl, or benzimidazolyl,
wherein each X is optionally substituted by one or more of the following: cyano, halo, a C₁₋₄ alkyl group optionally substituted by one or more fluoro, a C₁₋₄ alkoxy group optionally substituted by one or more fluoro, or a group CONR^{a}R^{b} in which R^{a} and R^{b} independently represent a C₁₋₃ alkyl group,
Y is OCH₂ or SCH₂ (wherein the heteroatom is connected to X), CH₂CH₂ or CH═CH,
R¹ is hydrogen or methyl,
A represents (CH₂)ₙ, wherein n is 0 or 1 and B represents (CH₂)ₘ, wherein m is 0 or 1,
R² represents H or, when A and B are identical and represents CH₂, R₂ represents H or F,
Z is phenyl or a heterocyclic group selected from thienyl, furyl, pyrrolyl wherein each Z is optionally substituted by cyano, fluoro, chloro or trifluoromethyl,
W represents phenyl or a heterocyclic group selected from thienyl, furyl, pyridyl, pyrazinyl, pyridazinyl, pyrrolyl, imidazolyl, thiazolyl, isothiazolyl, thiadiazolyl, pyrimidinyl, pyrazolyl, oxazolyl, isoxazolyl wherein each W is optionally substituted by one or more of the following: cyano, fluoro, chloro, trifluoromethoxy, difluoromethoxy or trifluoromethyl, or with one trifluoromethylsulfonyl or one 2,2-difluoro-1,3-dioxolane ring (fused with two adjacent aromatic carbon atoms in W),
as well as pharmaceutically acceptable salts, thereof.

4. A compound according to claim 1, wherein X represents a phenyl or pyridyl group optionally substituted by one or more halogen and is further substituted by a phenyl, phenoxy, 2-pyridyl or 3-pyridyl group, wherein the substituents (i.e. phenyl, phenoxy, 2-pyridyl or 3-pyridyl) may optionally be further substituted by one or more fluoro, chloro or cyano
Y is OCH₂ or SCH₂ (wherein the heteroatom is connected to X), CH₂CH₂ or CH═CH,
R¹ is hydrogen or methyl,
A represents (CH₂)ₙ, wherein n is 0 or 1 and B represents (CH₂)ₘ, wherein m is 0 or 1,
R² represents H or, when A and B are identical and represents CH₂, R₂ represents H or F,
Z is phenyl or a heterocyclic group selected from thienyl, furyl, pyrrolyl wherein each Z is optionally substituted by cyano, fluoro, chloro or trifluoromethyl,
W represents phenyl or a heterocyclic group selected from thienyl, furyl, pyridyl, pyrazinyl, pyridazinyl, pyrrolyl, imidazolyl, thiazolyl, isothiazolyl, thiadiazolyl, pyrimidinyl, pyrazolyl, oxazolyl, isoxazolyl wherein each W is optionally substituted by one or more of the following: cyano, fluoro, chloro, trifluoromethoxy, difluoromethoxy or trifluoromethyl, or with one trifluoromethylsulfonyl or one 2,2-difluoro-1,3-dioxolane ring (fused with two adjacent aromatic carbon atoms in W),
as well as pharmaceutically acceptable salts, thereof.

5. A compound according to claim 1, in which X represents a phenyl group substituted with one or more cyano, fluoro, chloro, trifluoromethoxy, difluoromethoxy or trifluoromethyl, or X represents a diphenylmethyl or a dipyridinomethyl group, optionally substituted at the aryl group(s) by one or more cyano, fluoro, chloro, trifluoromethoxy, difluoromethoxy or trifluoromethyl,
Y is OCH₂ (in which the heteroatom is connected to X),
R¹ is hydrogen,
A represents (CH₂)ₙ, wherein n is 0 or 1 and B represents (CH₂)ₘ, wherein m is 0 or 1,
R² represents H or, when A and B are identical and represents CH₂, R₂ represents H or F,
Z is thienyl, furyl or pyrrolyl,
W represents phenyl or a heterocyclic group selected from pyridyl, pyrazinyl, pyridazinyl, pyrrolyl, imidazolyl, thiazolyl, isothiazolyl, thiadiazolyl, pyrimidinyl, pyrazolyl, oxazolyl, isoxazolyl wherein each W is optionally substituted by one or more of the following: cyano, fluoro, chloro, trifluoromethoxy, difluoromethoxy or trifluoromethyl, or with one trifluoromethylsulfonyl or one 2,2-difluoro-1,3-dioxolane ring (fused with two adjacent aromatic carbon atoms in W),
as well as pharmaceutically acceptable salts thereof.

6. A compound according to claim 1, in which X represents a phenyl group substituted with one or more cyano, fluoro, chloro, trifluoromethoxy, difluoromethoxy or trifluoromethyl, or X represents a diphenylmethyl group, optionally substituted at the phenyl group(s) by one or more cyano, fluoro, chloro, trifluoromethoxy, difluoromethoxy or trifluoromethyl,
Y is OCH₂ (in which the heteroatom is connected to X),
R¹ is hydrogen,
A represents (CH₂)ₙ, wherein n is 0 or 1 and B represents (CH₂)ₘ, wherein m is 0 or 1,
R² represents H or, when A and B are identical and represents CH₂, R₂ represents H or F,
Z is 2,5-thienyl (where position 2 is linked to group W),
W represents phenyl or a heterocyclic group selected from pyridyl, pyrazinyl, pyridazinyl, pyrrolyl, imidazolyl, thiazolyl, isothiazolyl, thiadiazolyl, pyrimidinyl, pyrazolyl, oxazolyl, isoxazolyl wherein each W is optionally substituted by one or more of the following: cyano, fluoro, chloro, trifluoromethoxy, difluoromethoxy or trifluoromethyl, or with one trifluoromethylsulfonyl or one 2,2-difluoro-1,3-dioxolane ring (fused with two adjacent aromatic carbon atoms in W),
as well as pharmaceutically acceptable salts thereof.

7. A compound according to claim 1, in which X represents a phenyl group substituted with one or more cyano, fluoro, chloro, trifluoromethoxy, difluoromethoxy or trifluoromethyl, or X represents a diphenylmethyl group, optionally substituted at the phenyl group(s) by one or more cyano, fluoro, chloro, trifluoromethoxy, difluoromethoxy or trifluoromethyl,
Y is OCH₂ (in which the heteroatom is connected to X),
R¹ is hydrogen,
A represents (CH₂)ₙ, wherein n is 0 or 1 and B represents (CH₂)ₘ, wherein m is 0 or 1,
R² represents H or, when A and B are identical and represents CH₂, R₂ represents H or F,
Z is 2,5-furyl (where position 2 is linked to group W),
W represents phenyl or a heterocyclic group selected from pyridyl, pyrazinyl, pyridazinyl, pyrrolyl, imidazolyl, thiazolyl, isothiazolyl, thiadiazolyl, pyrimidinyl, pyrazolyl, oxazolyl, isoxazolyl wherein each W is optionally substituted by one or more of the following: cyano, fluoro, chloro, trifluoromethoxy, difluoromethoxy or trifluoromethyl, or with one trifluoromethylsulfonyl or one 2,2-difluoro-1,3-dioxolane ring (fused with two adjacent aromatic carbon atoms in W),
as well as pharmaceutically acceptable salts thereof.

8. A compound according to claim 1, in which X represents a phenyl group substituted with one or more cyano, fluoro, chloro, trifluoromethoxy, difluoromethoxy or trifluoromethyl, or X represents a diphenylmethyl group, optionally substituted (at the phenyl group(s)) by one or more cyano, fluoro, chloro, trifluoromethoxy, difluoromethoxy or trifluoromethyl,
Y is OCH₂ (in which the heteroatom is connected to X),
R¹ is hydrogen,
A represents (CH₂)ₙ, wherein n is 0 or 1 and B represents (CH₂)ₘ, wherein m is 0 or 1,
R² represents H or, when A and B are identical and represents CH₂, R₂ represents H or F,
Z is 1, 3-1H pyrrolyl (in which the heteroatom is connected to W),
W represents phenyl or a heterocyclic group selected from pyridyl, pyrazinyl, pyridazinyl, pyrrolyl, imidazolyl, thiazolyl, isothiazolyl, thiadiazolyl, pyrimidinyl, pyrazolyl, oxazolyl, isoxazolyl wherein each W is optionally substituted by one or more of the following: cyano, fluoro, chloro, trifluoromethoxy, difluoromethoxy or trifluoromethyl, or with one trifluoromethylsulfonyl or one 2,2-difluoro-1,3-dioxolane ring (fused with two adjacent aromatic carbon atoms in W),
as well as pharmaceutically acceptable salts thereof.

9. A compound according to claim 1, in which Z is pyrrolyl.

10. A compound according to claim 1, in which Z is 1, 3-1H pyrrolyl (in which the heteroatom is connected to W).

11. A compound according to claim 1, in which W is phenyl or 2-pyridyl, optionally substituted by one or more of the following: cyano, fluoro, chloro, trifluoromethoxy, difluoromethoxy, trifluoromethyl or trifluoromethylsulfonyl.

12. A compound according to claim 1, in which Y is OCH₂.

13. One or more of the following compounds:
2-(3-chlorophenoxy)-N-[1-[(1-phenyl-1H-pyrrol-3-yl)methyl]piperidin-4-yl}acetamide
2-(3-chlorophenoxy)-N-[1-({1-[4-(trifluoromethyl)phenyl]-1H-pyrrol-3-yl}methyl)piperidin-4-yl]acetamide
2-(3-chlorophenoxy)-N-(1-{[1-(4-methoxyphenyl)-1H-pyrrol-3-yl]methyl}piperidin-4-yl)acetamide
2-(3-chlorophenoxy)-N-(1-{[1-(2-chlorophenyl)-1H-pyrrol-3-yl]methyl}piperidin-4-yl)acetamide
2-(3-chlorophenoxy)-N-[1-({1-[5-(trifluoromethyl)pyridin-2-yl]-1H-pyrrol-3-yl}methyl)piperidin-4-yl]acetamide
2-(3-chlorophenoxy)-N-(1-{[1-(3-chlorophenyl)-1H-pyrrol-3-yl]methyl}piperidin-4-yl)acetamide
2-(3-chlorophenoxy)-N-[1-(4-pyridin-2-ylbenzyl)piperidin-4-yl]acetamide
2-(3-chlorophenoxy)-N-(1-{[5-(4-chlorophenyl)-2-furyl]methyl}piperidin-4-yl)acetamide
2-(3-chlorophenoxy)-N-[1-({1-[4-(trifluoromethoxy)phenyl]-1H-pyrrol-3-yl}methyl)piperidin-4-yl]acetamide
2-(3-chlorophenoxy)-N-{1-[3-(1H-pyrrol-1-yl)benzyl]piperidin-4-yl}acetamide
2-(3-chlorophenoxy)-N-[1-(3-pyridin-2-ylbenzyl)piperidin-4-yl]acetamide
2-(3-chlorophenoxy)-N-(1-{[5-(2,4-dichlorophenyl)-2-furyl]methyl}piperidin-4-yl)acetamide
2-(3-chlorophenoxy)-N-[1-({5-[1-methyl-5-(trifluoromethyl)-1H-pyrazol-3-yl]-2-thienyl}methyl)piperidin-4-yl]acetamide
N-(1-{[1-(4-bromophenyl)-1H-pyrrol-3-yl]methyl}piperidin-4-yl)-2-(3-chlorophenoxy)acetamide
2-(3-chlorophenoxy)-N-methyl-N-[1-({1-[4-(trifluoromethyl)phenyl]-1H-pyrrol-3-yl}methyl)piperidin-4-yl]acetamide
2-[(3-chlorophenyl)thio]-N-[1-({1-[4-(trifluoromethyl)phenyl]-1H-pyrrol-3-yl}methyl)piperidin-4-yl]acetamide
2-(pyridin-3-yloxy)-N-[1-({1-[4-(trifluoromethyl)phenyl]-1H-pyrrol-3-yl}methyl)piperidin-4-yl]acetamide
2-[3-(trifluoromethoxy)phenoxy]-N-[1-({1-[4-(trifluoromethyl)phenyl]-1H-pyrrol-3-yl}methyl)piperidin-4-yl]acetamide
2-[3-(trifluoromethoxy)phenoxy]-N-[1-({1-[5-(trifluoromethyl)pyridin-2-yl]-1H-pyrrol-3-yl}methyl)piperidin-4-yl]acetamide
2-(3-cyanophenoxy)-N-[1-({1-[4-(trifluoromethyl)phenyl]-1H-pyrrol-3-yl}methyl)piperidin-4-yl]acetamide
2-(3-fluorophenoxy)-N-[1-({1-[4-(tiifluoromethyl)phenyl]-1H-pyrrol-3-yl}methyl)piperidin-4-yl]acetamide
2-(3-cyanophenoxy)-N-[1-({5-[1-methyl-5-(trifluoromethyl)-1H-pyrazol-3-yl]-2-thienyl}methyl)piperidin-4-yl]acetamide
2-(2-chlorophenoxy)-N-[1-({1-[4-(trifluoromethyl)phenyl]-1H-pyrrol-3-yl}methyl)piperidin-4-yl]acetamide
2-(3-chlorophenoxy)-N-[1-({5-[4-(trifluoromethoxy)phenyl]-2-furyl}methyl)piperidin-4-yl]acetamide
2-(3-chlorophenoxy)-N-(1-{[1-(4-cyanophenyl)-1H-pyrrol-3-yl]methyl}piperidin-4-yl)acetamide
2-(3-cyanophenoxy)-N-(1-{[5-(2,4-dichlorophenyl)-2-furyl]methyl}piperidin-4-yl)acetamide
2-(3-cyanophenoxy)-N-[1-({1-[4-(trifluoromethoxy)phenyl]-1H-pyrrol-3-yl}methyl)piperidin-4-yl]acetamide
2-(3-chlorophenoxy)-N-(1-{[1-(5-chloropyrimidin-2-yl)-1H-pyrrol-3-yl]methyl}piperidin-4-yl)acetamide
3-(3-chlorophenyl)-N-[1-({1-[4-(trifluoromethyl)phenyl]-1H-pyrrol-3-yl}methyl)piperidin-4-yl]propanamide
(2E)-3-(3-chlorophenyl)-N-[1-({1-[4-(trifluoromethyl)phenyl]-1H-pyrrol-3-yl}methyl)piperidin-4-yl]acrylamide
2-(3,5-difluorophenoxy)-N-[1-({1-[4-(trifluoromethyl)phenyl]-1H-pyrrol-3-yl}methyl)piperidin-4-yl]acetamide
2-(2,6-diisopropylphenoxy)-N-[1-({1-[4-(trifluoromethyl)phenyl]-1H-pyrrol-3-yl}methyl)piperidin-4-yl]acetamide
2-(3-isopropylphenoxy)-N-[1-({1-[4-(trifluoromethyl)phenyl]-1H-pyrrol-3-yl}methyl)piperidin-4-yl]acetamide
2-(2-cyanophenoxy)-N-[1-({1-[4-(trifluoromethyl)phenyl]-1H-pyrrol-3-yl}methyl)piperidin-4-yl]acetamide
2-(isoquinolin-5-yloxy)-N-[1-({1-[4-(trifluoromethyl)phenyl]-1H-pyrrol-3-yl}methyl)piperidin-4-yl]acetamide
2-(3,4-difluorophenoxy)-N-[1-({1-[4-(trifluoromethyl)phenyl]-1H-pyrrol-3-yl}methyl)piperidin-4-yl]acetamide
2-[(5-chloropyridin-2-yl)oxy]-N-[1-({1-[4-(trifluoromethyl)phenyl]-1H-pyrrol-3-yl}methyl)piperidin-4-yl]acetamide
2-(3-chlorophenoxy)-N-[1-({1-[6-(trifluoromethyl)pyridin-3-yl]-1H-pyrrol-3-yl}methyl)piperidin-4-yl]acetamide
2-(biphenyl-3-yloxy)-N-[1-({1-[4-(trifluoromethyl)phenyl]-1H-pyrrol-3-yl}methyl)piperidin-4-yl]acetamide,
2-(4-chlorophenoxy)-2-methyl-N-[1-({1-[4-(trifluoromethyl)phenyl]-1H-pyrrol-3-yl}methyl)piperidin-4-yl]propanamide,
2-(3-chlorophenoxy)-N-[1-({1-[4-(trifluoromethyl)phenyl]-1H-pyrrol-3-yl}methyl)azetidin-3-yl]acetamide
2-(diphenylmethoxy)-N-[1-({1-[4-(trifluoromethyl)phenyl]-1H-pyrrol-3-yl}methyl)piperidin-4-yl]acetamide
2-(3-chlorophenoxy)-N-[(3S,4S)-3-fluoro-1-({1-[4-(trifluoromethyl)phenyl]-1H-pyrrol-3-yl}methyl)piperidin-4-yl]acetamide,
2-(3-chlorophenoxy)-N-[(3R,4R)-3-fluoro-1-({1-[4-(trifluoromethyl)phenyl]-1H-pyrrol-3-yl}methyl)piperidin-4-yl]acetamide
2-(3,4-difluorophenoxy)-N-[1-({1-[4-(trifluoromethyl)phenyl]-1H-pyrrol-3-yl}methyl)pyrrolidin-3-yl]acetamide
2-(3-chlorophenoxy)-N-{1-[(1-{4-[(trifluoromethyl)sulfonyl]phenyl}-1H-pyrrol-3-yl)methyl]piperidin-4-yl}acetamide
2-(3-chlorophenoxy)-N-(1-{[1-(2,2-difluoro-1,3-benzodioxol-5-yl)-1H-pyrrol-3-yl]methyl}piperidin-4-yl)acetamide
and pharmaceutically acceptable salts thereof.

14. A compound of formula Ia 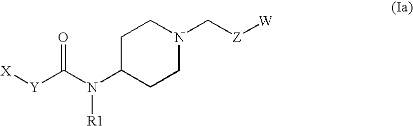
wherein X represents a 5-10 membered aryl or a heterocyclic group selected from pyrrolyl, imidazolyl, furyl, thienyl, thiazolyl, isothiazolyl, thiadiazolyl, pyrazolyl, oxazolyl, isoxazolyl, pyridyl, pyrazinyl, pyrimidinyl, pyridazinyl, quinolinyl, isoquinolyl, quinazolyl, indolyl, benzofuranyl, benzo[b]thienyl or benzimidazolyl,
wherein each X is optionally substituted by one or more of the following: cyano, halo, a C₁₋₄ alkyl group optionally substituted by one or more fluoro, a C₁₋₄ alkoxy group optionally substituted by one or more fluoro, a group CONR^{a}R^{b} in which R^{a} and R^{b} independently represent a C₁₋₃ alkyl group, phenyl, phenoxy, 2-pyridyl or 3-pyridyl, wherein the aromatic substituents (i.e. phenyl, phenoxy, 2-pyridyl or 3-pyridyl) may optionally be substituted by fluoro, chloro or cyano,
Y is OCH₂, SCH₂ (both in which the heteroatom is connected to X), CH₂CH₂ or CH═CH, wherein each carbon in Y is optionally substituted by 1 or 2 methyl groups and/or 1 or 2 fluoro,
R¹ represents H or a C₁₋₄alkyl group,
Z represents phenyl or a heterocyclic group selected from thienyl, furyl, pyridyl, pyrazinyl, pyridazinyl, pyrrolyl, imidazolyl, thiazolyl, isothiazolyl, thiadiazolyl, pyrimidinyl, pyrazolyl, oxazolyl, isoxazolyl wherein each Z is optionally substituted by one or more of the following: cyano, halo, a C₁₋₄ alkyl group optionally substituted by one or more fluoro, a C₁₋₄ alkoxy group optionally substituted by one or more fluoro,
W represents phenyl or a heterocyclic group selected from thienyl, furyl, pyridyl, pyrazinyl, pyridazinyl, pyrrolyl, imidazolyl, thiazolyl, isothiazolyl, thiadiazolyl, pyrimidinyl, pyrazolyl, oxazolyl, isoxazolyl wherein each W is optionally substituted by one or more of the following: cyano, halo, a C₁₋₄ alkyl group optionally substituted by one or more fluoro, a C₁₋₄ alkoxy group optionally substituted by one or more fluoro,
as well as tautomers, optical isomers and racemates thereof as well as pharmaceutically acceptable salts thereof,
with the proviso that 2-(4-chlorophenoxy)-N-{1-[4-(1,2,3-thiadiazol-4-yl)benzyl]piperidin-4-yl}acetamide is excluded.

15. A compound according to claim 14, in which X represents a phenyl or pyridyl group substituted with one or more cyano, fluoro, chloro, trifluoromethoxy, difluoromethoxy or trifluoromethyl,
Y is OCH₂ or SCH₂ (both in which the heteroatom is connected to X) CH₂CH₂ or CH═CH,
R¹ is hydrogen or methyl,
Z is phenyl or a heterocyclic group selected from thienyl, furyl, pyridyl, pyrrolyl wherein each Z is optionally substituted by cyano, fluoro, chloro or trifluoromethyl,
W represents phenyl or a heterocyclic group selected from thienyl, furyl, pyridyl, pyrazinyl, pyridazinyl, pyrrolyl, imidazolyl, thiazolyl, isothiazolyl, thiadiazolyl, pyrimidinyl, pyrazolyl, oxazolyl, isoxazolyl wherein each W is optionally substituted by one or more of the following: cyano, fluoro, chloro, trifluoromethoxy, difluoromethoxy or trifluoromethyl,
as well as pharmaceutically acceptable salts, thereof.

16. A compound according to claim 1, wherein X represents naphthyl or a heteroaryl ring selected from quinolinyl, isoquinolyl, quinazolyl, indolyl, benzofuranyl, benzo[b]thienyl, or benzimidazolyl,
wherein each X is optionally substituted by one or more of the following: cyano, halo, a C₁₋₄ alkyl group optionally substituted by one or more fluoro, a C₁₋₄ alkoxy group optionally substituted by one or more fluoro, a group CONR^{a}R^{b} in which R^{a} and R^{b} independently represent a C₁₋₃ alkyl group,
Y is OCH₂ or SCH₂ (both in which the heteroatom is connected to X) CH₂CH₂ or CH═CH,
R¹ is hydrogen or methyl,
Z is phenyl or a heterocyclic group selected from thienyl, furyl, pyridyl, pyrrolyl wherein each Z is optionally substituted by cyano, fluoro, chloro or trifluoromethyl,
W represents phenyl or a heterocyclic group selected from thienyl, furyl, pyridyl, pyrazinyl, pyridazinyl, pyrrolyl, imidazolyl, thiazolyl, isothiazolyl, thiadiazolyl, pyrimidinyl, pyrazolyl, oxazolyl, isoxazolyl wherein each W is optionally substituted by one or more of the following: cyano, fluoro, chloro, trifluoromethoxy, difluoromethoxy or trifluoromethyl,
as well as pharmaceutically acceptable salts, thereof.

17. A compound according to claim 14, wherein X represents phenyl or pyridyl group optionally substituted by one or more halogen and is further substituted by a phenyl, phenoxy, 2-pyridyl or 3-pyridyl group, wherein the substituents (i.e. phenyl, phenoxy, 2-pyridyl or 3-pyridyl) may optionally be further substituted by on or more fluoro, chloro or cyano,
Y is OCH₂ or SCH₂ (both in which the heteroatom is connected to X) CH₂CH₂ or CH═CH,
R¹ is hydrogen or methyl,
Z is phenyl or a heterocyclic group selected from thienyl, furyl, pyridyl, pyrrolyl wherein each Z is optionally substituted by cyano, fluoro, chloro or trifluoromethyl,
W represents phenyl or a heterocyclic group selected from thienyl, furyl, pyridyl, pyrazinyl, pyridazinyl, pyrrolyl, imidazolyl, thiazolyl, isothiazolyl, thiadiazolyl, pyrimidinyl, pyrazolyl, oxazolyl, isoxazolyl wherein each W is optionally substituted by one or more of the following: cyano, fluoro, chloro, trifluoromethoxy, difluoromethoxy or trifluoromethyl,
as well as pharmaceutically acceptable salts, thereof.

18. A compound according to claim 14, in which X represents a phenyl group substituted with one or more cyano, fluoro, chloro, trifluoromethoxy, difluoromethoxy or trifluoromethyl,
Y is OCH₂ (in which the heteroatom is connected to X),
R¹ is hydrogen,
Z is thienyl, furyl or pyrrolyl,
W represents phenyl or a heterocyclic group selected from pyridyl, pyrazinyl, pyridazinyl, pyrrolyl, imidazolyl, thiazolyl, isothiazolyl, thiadiazolyl, pyrimidinyl, pyrazolyl, oxazolyl, isoxazolyl wherein each W is optionally substituted by one or more of the following: cyano, fluoro, chloro, trifluoromethoxy, difluoromethoxy or trifluoromethyl,
as well as pharmaceutically acceptable salts thereof.

19. A compound according to claim 14, in which X represents a phenyl group substituted with one or more cyano, fluoro, chloro, trifluoromethoxy, difluoromethoxy or trifluoromethyl,
Y is OCH₂ (in which the heteroatom is connected to X),
R¹ is hydrogen,
Z is 2,5-thienyl (where position 2 is linked to group W),
W represents phenyl or a heterocyclic group selected from pyridyl, pyrazinyl, pyridazinyl, pyrrolyl, imidazolyl, thiazolyl, isothiazolyl, thiadiazolyl, pyrimidinyl, pyrazolyl, oxazolyl, isoxazolyl wherein each W is optionally substituted by one or more of the following: cyano, fluoro, chloro, trifluoromethoxy, difluoromethoxy or trifluoromethyl,
as well as pharmaceutically acceptable salts thereof.

20. A compound according to claim 14, in which X represents a phenyl group substituted with one or more cyano, fluoro, chloro, trifluoromethoxy, difluoromethoxy or trifluoromethyl,
Y is OCH₂ (in which the heteroatom is connected to X),
R¹ is hydrogen,
Z is 2,5-furyl (where position 2 is linked to group W),
W represents phenyl or a heterocyclic group selected from pyridyl, pyrazinyl, pyridazinyl, pyrrolyl, imidazolyl, thiazolyl, isothiazolyl, thiadiazolyl, pyrimidinyl, pyrazolyl, oxazolyl, isoxazolyl wherein each W is optionally substituted by one or more of the following: cyano, fluoro, chloro, trifluoromethoxy, difluoromethoxy or trifluoromethyl,
as well as pharmaceutically acceptable salts thereof.

21. A compound according to claim 14, in which X represents a phenyl group substituted with one or more cyano, fluoro, chloro, trifluoromethoxy, difluoromethoxy or trifluoromethyl,
Y is OCH₂ (in which the heteroatom is connected to X),
R¹ is hydrogen,
Z is 1, 3-1H pyrrolyl (in which the heteroatom is connected to W),
W represents phenyl or a heterocyclic group selected from pyridyl, pyrazinyl, pyridazinyl, pyrrolyl, imidazolyl, thiazolyl, isothiazolyl, thiadiazolyl, pyrimidinyl, pyrazolyl, oxazolyl, isoxazolyl wherein each W is optionally substituted by one or more of the following: cyano, fluoro, chloro, trifluoromethoxy, difluoromethoxy or trifluoromethyl,
as well as pharmaceutically acceptable salts thereof.

22. A compound according to claim 14, in which Z is pyrrolyl.

23. A compound according to claim 14, in which Z is 1, 3-1H pyrrolyl (in which the heteroatom is connected to W).

24. A compound according to claim 14, in which W is phenyl or 2-pyridyl, optionally substituted by one or more of the following: cyano, fluoro, chloro, trifluoromethoxy, difluoromethoxy or trifluoromethyl.

25. A compound according to claim 14, in which Y is OCH₂.

26. (canceled)

27. A pharmaceutical formulation comprising a compound of claim 1 and a pharmaceutically acceptable adjuvant, diluent or carrier.

28-29. (canceled)

30. A process for the preparation of a compound of formula I or formula Ia comprising
reacting a compound of formula II with a compound of formula III 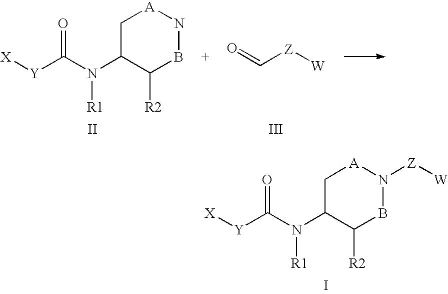
wherein X represents phenyl, naphthyl, pyrrolyl, imidazolyl, furyl, thienyl, thiazolyl, isothiazolyl, thiadiazolyl, pyrazolyl, oxazolyl, isoxazolyl, pyridyl, pyrazinyl, pyrimidinyl, pyridazinyl, quinolinyl, isoquinolyl, quinazolyl, indolyl, benzofuranyl, benzo[b]thienyl or benzimidazolyl,
wherein each X is optionally substituted by one or more of the following: cyano, halo, a C₁₋₄ alkyl group optionally substituted by one or more fluoro, a C₁₋₄alkoxy group optionally substituted by one or more fluoro, a group CONR^{a}R^{b} in which R^{a} and R^{b} independently represent a C₁₋₃alkyl group, phenyl, phenoxy, 2-pyridyl or 3-pyridyl, wherein the aromatic substituents (i.e. phenyl, phenoxy, 2-pyridyl or 3-pyridyl) may optionally be substituted by fluoro, chloro or cyano, or
X represents a diphenylmethyl or a dipyridinylmethyl group, optionally independently substituted at the aryl group(s) by one or more cyano, halo, trifluoromethoxy, difluoromethoxy or trifluoromethyl,
Y is OCH₂, SCH₂ (wherein the heteroatom is connected to X), CH₂CH₂ or CH═CH, wherein each carbon in Y is optionally substituted by 1 or 2 methyl groups and/or 1 or 2 fluoro,
R¹ represents H or a C₁₋₄alkyl group,
A represents (CH₂)ₙ, wherein n is 0 or 1 and B represents (CH₂)ₘ, wherein m is 0 or 1,
R² represents H or, when A and B are identical and represents CH₂, R₂ represents H or F,
Z represents phenyl or a heterocyclic group selected from thienyl, furyl, pyridyl, pyrazinyl, pyridazinyl, pyrrolyl, imidazolyl, thiazolyl, isothiazolyl, thiadiazolyl, pyrimidinyl, pyrazolyl, oxazolyl, isoxazolyl wherein each Z is optionally substituted by one or more of the following: cyano, halo, a C₁₋₄alkyl group optionally substituted by one or more fluoro, a C₁₋₄alkoxy group optionally substituted by one or more fluoro, and
W represents phenyl or a heterocyclic group selected from thienyl, furyl, pyridyl, pyrazinyl, pyridazinyl, pyrrolyl, imidazolyl, thiazolyl, isothiazolyl, thiadiazolyl, pyrimidinyl, pyrazolyl, oxazolyl, isoxazolyl wherein each W is optionally substituted by one or more of the following: cyano, halo, a C₁₋₄alkyl group optionally substituted by one or more fluoro, a C₁₋₄alkoxy group optionally substituted by one or more fluoro, or W is optionally substituted with a trifluoromethylsulfonyl or a 2,2-difluoro-1,3-dioxolane ring (fused with two adjacent aromatic carbon atoms in W).

31. A process for the preparation of a compound of formula I or formula Ia comprising
reacting a compound of formula IV with a compound of formula V 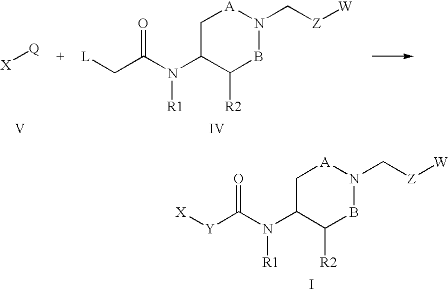
wherein X represents phenyl, naphthyl, pyrrolyl, imidazolyl, furyl, thienyl, thiazolyl, isothiazolyl, thiadiazolyl, pyrazolyl, oxazolyl, isoxazolyl, pyridyl, pyrazinyl, pyrimidinyl, pyridazinyl, quinolinyl, isoquinolyl, quinazolyl, indolyl, benzofuranyl, benzo[b]thienyl or benzimidazolyl,
wherein each X is optionally substituted by one or more of the following: cyano, halo, a C₁₋₄ alkyl group optionally substituted by one or more fluoro, a C₁₋₄alkoxy group optionally substituted by one or more fluoro, a group CONR^{a}R^{b} in which R^{a} and R^{b} independently represent a C₁₋₃alkyl group, phenyl, phenoxy, 2-pyridyl or 3-pyridyl, wherein the aromatic substituents (i.e. phenyl, phenoxy, 2-pyridyl or 3-pyridyl) may optionally be substituted by fluoro, chloro or cyano, or
X represents a diphenylmethyl or a dipyridinylmethyl group, optionally independently substituted at the aryl group(s) by one or more cyano, halo, trifluoromethoxy, difluoromethoxy or trifluoromethyl,
Y is OCH₂, SCH₂ (wherein the heteroatom is connected to X), CH₂CH₂ or CH═CH, wherein each carbon in Y is optionally substituted by 1 or 2 methyl groups and/or 1 or 2 fluoro,
R¹ represents H or a C₁₋₄alkyl group,
A represents (CH₂)ₙ, wherein n is 0 or 1 and B represents (CH₂)ₘ, wherein m is 0 or 1,
R² represents H or, when A and B are identical and represents CH₂, R₂ represents H or F,
Z represents phenyl or a heterocyclic group selected from thienyl, furyl, pyridyl, pyrazinyl, pyridazinyl, pyrrolyl, imidazolyl, thiazolyl, isothiazolyl, thiadiazolyl, pyrimidinyl, pyrazolyl, oxazolyl, isoxazolyl wherein each Z is optionally substituted by one or more of the following: cyano, halo, a C₁₋₄alkyl group optionally substituted by one or more fluoro, a C₁₋₄alkoxy group optionally substituted by one or more fluoro,
W represents phenyl or a heterocyclic group selected from thienyl, furyl, pyridyl, pyrazinyl, pyridazinyl, pyrrolyl, imidazolyl, thiazolyl, isothiazolyl, thiadiazolyl, pyrimidinyl, pyrazolyl, oxazolyl, isoxazolyl wherein each W is optionally substituted by one or more of the following: cyano, halo, a C₁₋₄alkyl group optionally substituted by one or more fluoro, a C₁₋₄alkoxy group optionally substituted by one or more fluoro, or W is optionally substituted with a trifluoromethylsulfonyl or a 2,2-difluoro-1,3-dioxolane ring (fused with two adjacent aromatic carbon atoms in W),
Q represents a hydroxyl or a mercapto group, and
L represents a leaving group.

32. A process for the preparation of a compound of formula I or formula Ia comprising
reacting a compound of formula VI with a compound of formula VII 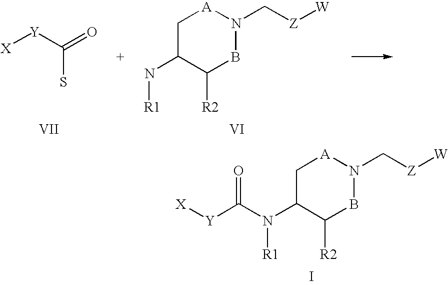
wherein X represents phenyl, naphthyl, pyrrolyl, imidazolyl, furyl, thienyl, thiazolyl, isothiazolyl, thiadiazolyl, pyrazolyl, oxazolyl, isoxazolyl, pyridyl, pyrazinyl, pyrimidinyl, pyridazinyl, quinolinyl, isoquinolyl, quinazolyl, indolyl, benzofuranyl, benzo[b]thienyl or benzimidazolyl,
wherein each X is optionally substituted by one or more of the following: cyano, halo, a C₁₋₄alkyl group optionally substituted by one or more fluoro, a C₁₋₄alkoxy group optionally substituted by one or more fluoro, a group CONR^{a}R^{b} in which R^{a} and R^{b} independently represent a C₁₋₃alkyl group, phenyl, phenoxy, 2-pyridyl or 3-pyridyl, wherein the aromatic substituents (i.e. phenyl, phenoxy, 2-pyridyl or 3-pyridyl) may optionally be substituted by fluoro, chloro or cyano, or
X represents a diphenylmethyl or a dipyridinylmethyl group, optionally independently substituted at the aryl group(s) by one or more cyano, halo, trifluoromethoxy, difluoromethoxy or trifluoromethyl,
Y is OCH₂, SCH₂ (wherein the heteroatom is connected to X), CH₂CH₂ or CH═CH, wherein each carbon in Y is optionally substituted by 1 or 2 methyl groups and/or 1 or 2 fluoro,
R¹ represents H or a C₁₋₄alkyl group,
A represents (CH₂)ₙ, wherein n is 0 or 1 and B represents (CH₂)ₘ, wherein m is 0 or 1,
R² represents H or, when A and B are identical and represents CH₂, R₂ represents H or F,
Z represents phenyl or a heterocyclic group selected from thienyl, furyl, pyridyl, pyrazinyl, pyridazinyl, pyrrolyl, imidazolyl, thiazolyl, isothiazolyl, thiadiazolyl, pyrimidinyl, pyrazolyl, oxazolyl, isoxazolyl wherein each Z is optionally substituted by one or more of the following: cyano, halo, a C₁₋₄alkyl group optionally substituted by one or more fluoro, a C₁₋₄alkoxy group optionally substituted by one or more fluoro,
W represents phenyl or a heterocyclic group selected from thienyl, furyl, pyridyl, pyrazinyl, pyridazinyl, pyrrolyl, imidazolyl, thiazolyl, isothiazolyl, thiadiazolyl, pyrimidinyl, pyrazolyl, oxazolyl, isoxazolyl wherein each W is optionally substituted by one or more of the following: cyano, halo, a C₁₋₄alkyl group optionally substituted by one or more fluoro, a C₁₋₄alkoxy group optionally substituted by one or more fluoro, or W is optionally substituted with a trifluoromethylsulfonyl or a 2,2-difluoro-1,3-dioxolane ring (fused with two adjacent aromatic carbon atoms in W), and
S represents a hydroxy group or a chlorine atom.

33. One or more of the following compounds:
2-(3-chlorophenoxy)-N-piperidin-4-ylacetamide
2-(3-cyanophenoxy)-N-piperidin-4-ylacetamide
2-(3-fluorophenoxy)-N-piperidin-4-ylacetamide
2-(2-chlorophenoxy)-N-piperidin-4-ylacetamide
N-piperidin-4-yl-2-(pyridin-3-yloxy)acetamide
N-piperidin-4-yl-2-[3-(trifluoromethoxy)phenoxy]acetamide
2-phenoxy-N-piperidin-4-ylacetamide
2-(3-chlorophenoxy)-N-methyl-N-piperidin-4-ylacetamide
2-[(3-chlorophenyl)thio]-N-piperidin-4-ylacetamide
1-[5-(trifluoromethyl)pyridin-2-yl]-1H-pyrrole-3-carbaldehyde
1-(5-chloropyrimidin-2-yl)-1H-pyrrole-3-carbaldehyde
4-(3-formyl-1H-pyrrol-1-yl)benzonitrile
2-chloro-N-[1-({1-[4-(trifluoromethyl)phenyl]-1H-pyrrol-3-yl}methyl)piperidin-4-yl]acetamide
1-({1-[4-(trifluoromethyl)phenyl]-1H-pyrrol-3-yl}methyl)piperidin-4-amine dihydrochloride
tert-butyl[1-({1-[4-(trifluoromethyl)phenyl]-1H-pyrrol-3-yl}methyl)piperidin-4-yl]carbamate
1-(6-trifluoromethyl-pyridin-3-yl)-1H-pyrrole-3-carbaldehyde
2-(3,4-difluorophenoxy)-N-pyrrolidin-3-ylacetamide
1-(2,2-difluoro-benzo[1,3]dioxol-5-yl)-1H-pyrrole-3-carbaldehyde and
1-(4-Trifluoromethanesulfonyl-phenyl)-1H-pyrrole-3-carbaldehyde.

34. A method of treating obesity, a psychiatric disorder, anxiety, an anxio-depressive disorder, depression, bipolar disorder, ADHD, a cognitive disorder, a memory disorder, schizophrenia, epilepsy, a neurological disorder, or a pain related disorder, comprising administering a pharmacologically effective amount of a compound according to claim 1 to a patient in need thereof.

35. A method of treating obesity, type II diabetes, metabolic syndrome or prevention of type II diabetes comprising administering a pharmacologically effective amount of a compound according to claim 1 to a patient in need thereof.

36. A pharmaceutical formulation comprising a compound of claim 14 and a pharmaceutically acceptable adjuvant, diluent or carrier.

37. A process according to claim 31 wherein the leaving group is a halo or methanesulfonyloxy.

38. A method of treating obesity, a psychiatric disorder, anxiety, an anxio-depressive disorder, depression, bipolar disorder, ADHD, a cognitive disorder, a memory disorder, schizophrenia, epilepsy, a neurological disorder, or a pain related disorder, comprising administering a pharmacologically effective amount of a compound according to claim 14 to a patient in need thereof.

39. A method of treating obesity, type II diabetes, metabolic syndrome or prevention of type II diabetes comprising administering a pharmacologically effective amount of a compound according to claim 14 to a patient in need thereof.
